(19) United States
(12) Patent Application Publication
(10) Pub. No.: US 2010/0063046 A1
(43) Pub. Date: Mar. 11, 2010

(54) TETRACYCLIC IMIDAZOLE ANALOGS

(76) Inventors: Jeffrey P. Whitten, Santee, CA (US); Michael Schwaebe, San Diego, CA (US)

Correspondence Address: MORRISON & FOERSTER LLP, 12531 HIGH BLUFF DRIVE, SUITE 100, SAN DIEGO, CA, 92130-2040, US

(21) Appl. No.: 12/301,090

(22) PCT Filed: May 17, 2007
(86) PCT No.: PCT/US2007/069186

§ 371 Date: Mar. 5, 2009

Related U.S. Application Data
(60) Provisional application No. 60/801,550, filed on May 17, 2006.

(51) Int. Cl. A61K 31/519; C07D 471/14; A61P 35/00; A61P 31/12; A61K 31/5377; C12N 5/00; C40B 30/00

(52) U.S. Cl. 514/233.2; 544/247; 544/115; 544/361; 514/252.16; 514/257; 435/375; 506/7

(57) ABSTRACT

The present invention provides tetracyclic imidazole analogs which may inhibit cell proliferation and/or induce cell apoptosis. The present invention also provides methods of preparing these compounds, and methods of using the same.

### FIELD OF THE INVENTION

The invention relates to tetracyclic imidazole analogs and uses thereof. The invention also relates to methods of preparing these compounds.

### BACKGROUND

Evidence suggests quadruplex DNA structures can exist in vivo in specific regions of the genome, including the telomeric ends of chromosomes and oncogene regulatory regions (Han, et al., Trends Pharm. Sci. (2000) 21:136-142). Quadruplex structures can form in purine-rich strands of nucleic acids. In duplex nucleic acids, certain purine rich strands are capable of engaging in a slow equilibrium between a typical duplex helix structure and an unwound and non-B-form regions. These unwound and non-B forms can be referred to as “paranemic structures.” Some forms are associated with sensitivity to S1 nuclease digestion, which can be referred to as “nuclease hypersensitivity elements” or “NHEs.” A quadruplex is one type of paranemic structure and certain NHEs can adopt a quadruplex structure. Such quadruplex structures are reported to be involved in the activities of certain anticancer drugs; thus compounds that modulate quadruplex formation can be useful in cancer therapy. See Y. He, et al., Nucleic Acids Res. vol. 32(18), 5359-67 (2004).

### SUMMARY OF THE INVENTION

The present invention provides imidazole analogs which may inhibit cell proliferation and/or induce cell apoptosis. The compounds comprise a tetracyclic core group that is linked to an amine functionality; the amine may be connected at any one of three positions on the tetracyclic core. The tetracyclic core comprises a six-membered ring fused to a pyrimidine-type ring, which is fused to an imidazole/imidazoline type ring; which is in turn fused to at least one additional ring. The amine group is linked to this core from either the pyrimidine-type ring or the imidazole.

The compounds of the invention exert biological activity in assays described herein. For example, compounds of the invention are cytotoxic in a cell viability assay described hereafter. Though not limiting the invention by any theory of its operation, it is believed that the compounds can function in part by interacting with quadruplex-forming regions of nucleic acids and modulating ribosomal RNA transcription. Compounds of the invention also may modulate the interaction of quadruplex-forming nucleic acids with nucleolin, a protein that is associated with apoptosis; thus modulation of the activity, localization or stability of nucleolin may also contribute to the ability of these compounds to induce apoptosis. The present invention also provides methods of preparing these compounds, and methods of using the same.

In one aspect, the present invention provides a compound of formula (1) or (2) or (3),

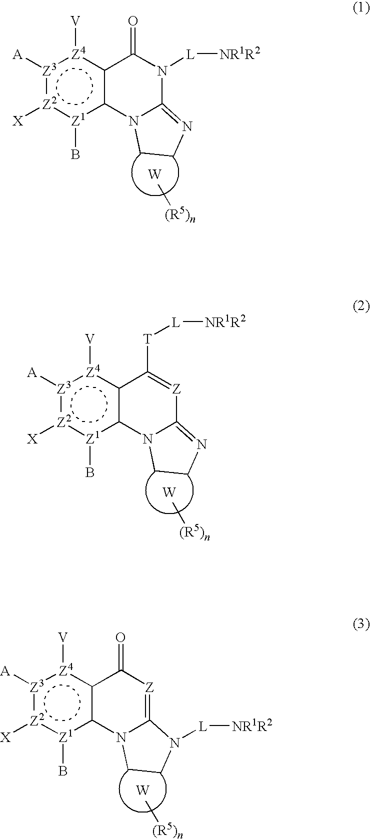

wherein each A, V, B, and X that is present is independently selected from H, halo, azido, CN, CF₃, CONR¹R², R², CH₂R², SR², OR²C(═O)R², and NR¹R², wherein in NR¹R², R¹ and R² can optionally cyclize to form an optionally substituted azacyclic group; or

wherein A and X, or A and V, or X and B may form a carbocyclic ring, heterocyclic ring, aryl or heteroaryl ring, each of which may be optionally substituted with one or two R³ groups and/or may be fused with an additional ring;

wherein in L-NR¹R², R¹ and R² taken together may form an optionally substituted azacyclic group, or R¹ or R² taken together with at least a portion of L may form an optionally substituted heterocyclic ring;

each Z is independently CH, CR³ or N;

each Z¹, Z², Z³, and Z⁴ is independently C or N, provided no two of them represent adjacent nitrogen atoms;

T is O, S(O)ₘ or NR⁴;

each R¹ is H or a C₁₋₁₀ alkyl or C₂₋₁₀ alkenyl group that can be substituted with one or more substituents selected from halo, ═O, OR², NR²₂, S(O)ₘR², COOR², and CONR²₂;

each m is independently 0-2;

each n is independently 0-4;

each R² is independently H or an optionally substituted C₁—10 alkyl or optionally substituted C₂₋₁₀ alkenyl optionally containing one or more non-adjacent heteroatoms selected from N, O, and S in place of carbon atoms, and optionally including a carbocyclic or heterocyclic ring; or R² is an optionally substituted carbocyclic, heterocyclic, 6-10 membered aryl or 5-14 membered heteroaryl ring containing one or more N, O or S;

each R³ is independently an optionally substituted group selected from C₁₋₆ alkyl, C₆₋₁₀ aryl, and C₅₋₁₂ heteroaryl, or R³ is selected from halo, nitro, OR′, SR′, SO₂R′, NR′₂, CN, CF₃, COOR′, and CONR′₂, wherein each R′ is independently H or C₁₋₆ alkyl and can optionally include one N, O or S in place of a carbon atom, or R³ can be L-NR¹R² or CON(R′)-L-NR¹R², wherein in NR¹R², R¹ and R² can optionally cyclize to form an optionally substituted azacyclic group;

each R⁴ is H or a C₁₋₁₀ alkyl or C₂₋₁₀ alkenyl group that can be substituted with one or more substituents selected from halo, ═O, OR², NR²₂, S(O)ₘR², COOR², and CONR²₂;

each L is a divalent hydrocarbon linker having up to five atoms counted along the shortest path between the two open valence, which linker may include one or two heteroatoms and may be substituted by one or more groups selected from halo, ═O, C₁₋₆ alkyl, OR′, SR′, SO₂R′, NR′₂, CN, CF₃, COOR′, and CONR′₂, wherein each R′ is independently H or C₁₋₆ alkyl;

each W represents an optionally substituted aryl or heteroaryl ring, which may be a monocyclic group with 5-6 ring atoms, or may be a 5-6 membered ring that is fused with or bonded to one or more additional aryl, heterocyclic, or heteroaryl rings; and

each R⁵ is a substituent at any position on W, and is selected from H, halo, CN, CF₃, OR², NR¹R², and C₁₋₆ alkyl and C₂₋₆ alkenyl, each optionally substituted by one or more substituents selected from halo, ═O, OR², S(O)ₘR², and NR¹R², wherein in NR¹R², R¹ and R² can optionally cyclize to form an optionally substituted azacyclic group; or R⁵ can be an inorganic substituent; or two adjacent R⁵ may be linked to form a 5-6 membered substituted or unsubstituted carbocyclic or heterocyclic ring, optionally fused to an additional substituted or unsubstituted carbocyclic or heterocyclic ring;

or a pharmaceutically acceptable salt thereof.

In the above formulas (1), (2) and (3), the five-membered ring containing two nitrogens is an imidazole or imidazoline derivative, and W represents a ring that includes two ring atoms of the imidazole/imidazoline ring shown. W is typically an optionally substituted 5- or 6-membered aromatic or heteroaromatic ring that is optionally fused to another substituted aryl or heteroaryl or heterocyclic or carbocyclic group. Frequently, W is selected from the group consisting of the following structures, in which the open valences (broken bonds) indicate where W attaches to each of the N atoms of the imidazole/imidazoline ring in formula (1), (2) or (3):

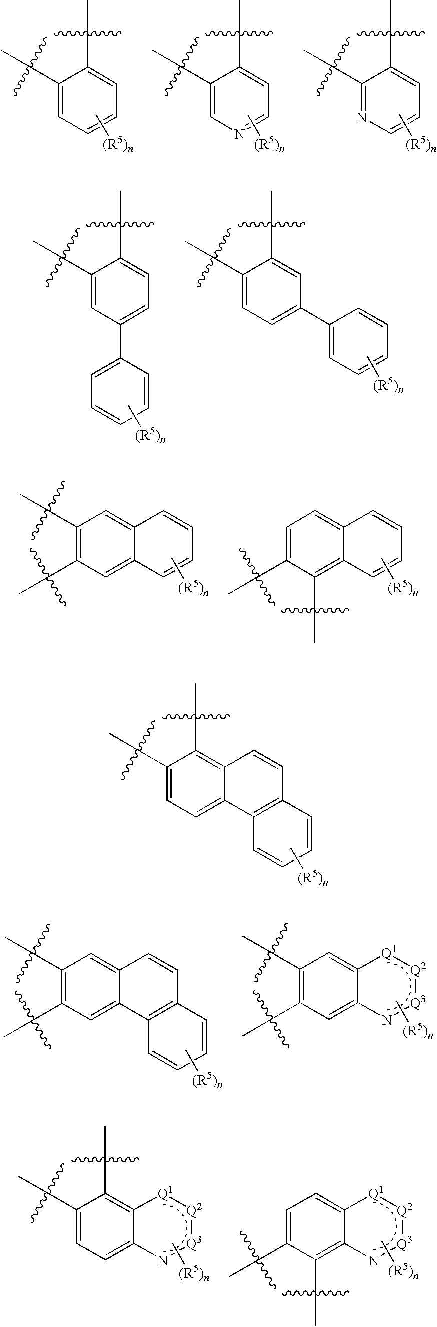

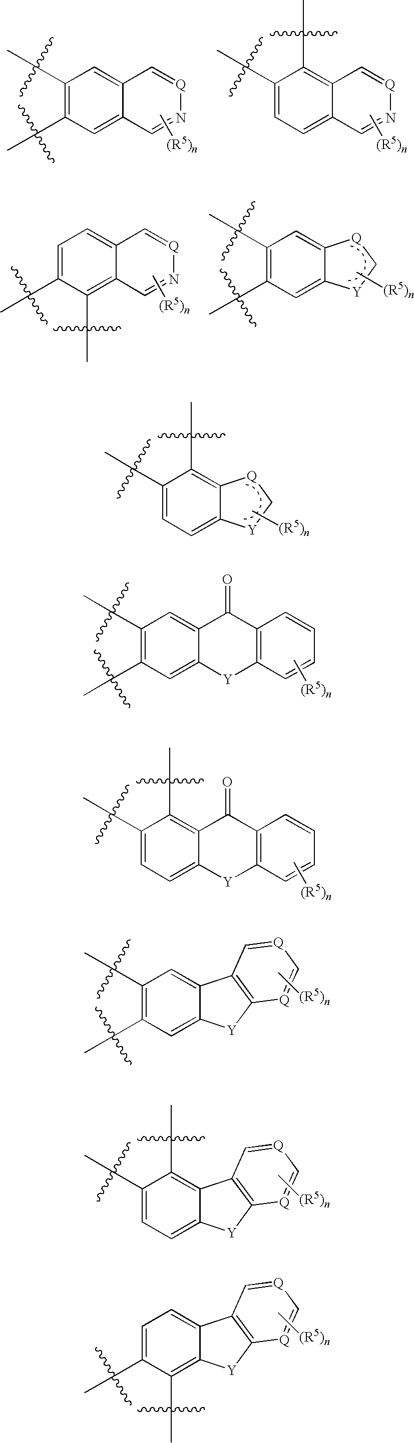

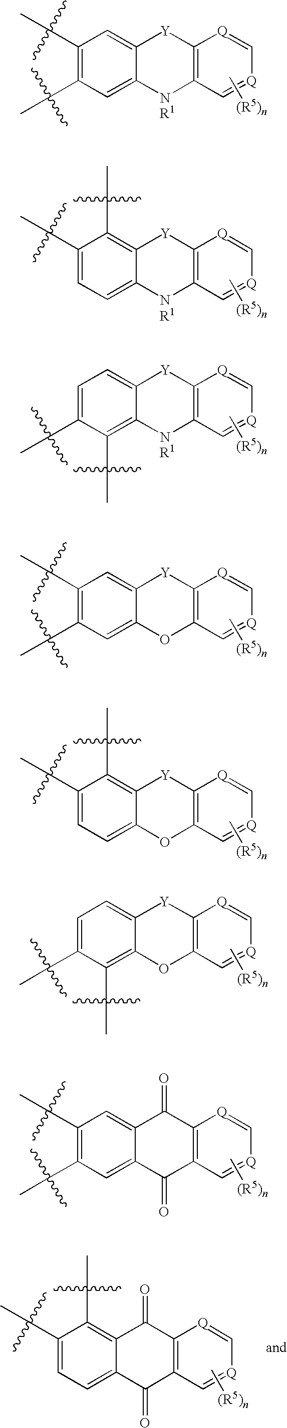

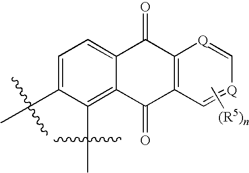

wherein each Q, Q¹, Q², and Q³ is independently CR³ or N;

each Y is independently O, CR¹₂, C═O or NR¹; and

n, R¹, R³ and R⁵ are as defined above.

In the multiple-ring structures above that represent specific embodiments of W, (R⁵) is depicted as though it is positioned on one particular ring even though the embodiment of W comprises multiple rings; that is for convenience of drawing, only, though, and it is understood that substituents represented by R⁵ may be on any available valence of any of the rings comprising W.

The compounds of formulas (1), (2) and (3) are characterized by a polycyclic core and an essential amine group represented by -L-NR¹R². The amine group -L-NR¹R² can be attached at one of three positions as represented by the three formulas, and in some embodiments, the compounds of the invention include a second -L-NR¹R² group as well, which can be attached to Z in formula (2) or formula (3) when that Z represents C, for example.

The -L-portion of -L-NR¹R² is a divalent hydrocarbon linker that may include a ring or ring system, and may be substituted. The two open valences on L (where L connects to the ring system shown in the formulas above, and where L connects to NR¹R²) are separated by up to ten atoms in some embodiments, and up to seven or up to five atoms in other embodiments, counted along the shortest path (fewest intervening atoms) separating the two open valence positions. In some embodiments, L is an alkylene group, which is one to ten carbons in length, or sometimes one to seven or at times one to five carbons in length, or two to five atoms in length, and which may contain one or two heteroatoms selected from N, O and S in place of one or two carbons of the alkylene group, provided that each of the terminal atoms of L is carbon. Thus L can be (CH₂)₁₋₅, or (CH₂)₂₋₄, and sometimes it is (CH₂)₃; or L can be CH₂—O—CH₂ or cyclohexan-1,4-diyl, for example. L may also be substituted with groups that are commonly used as substituents on alkyl groups, such as those described below.

The NR¹R² portion of this -L-NR¹R² group is often a basic amine group; for example, it can be a dialkyl amine such as dimethyl amine or diethyl amine, or it can be a cyclic group such as morpholine, piperidine, pyrrolidine, aziridine, azetidine, azepine, or piperazine when R¹ and R² are linked together. In each case, R¹ and R² of this L-NR¹R² group can be substituted with substituents including those described below. Preferably, the substituents on -L-NR¹R² do not include a carbonyl oxygen on any carbon atom that is directly linked to N.

R¹ or R² can cyclize onto L or a portion of L to form an optionally substituted heterocyclic ring having 3-8 ring members and optionally one additional heteroatom selected from N, O and S as a ring member in addition to the nitrogen of the NR¹R² group; or R¹ and R² can cyclize together to form an optionally substituted azacyclic group. In certain embodiments of the above molecules, NR¹R² represents pyrrolidine in at least one -L-NR¹R² group. In specific embodiments, -L-NR¹R² represents a group selected from:

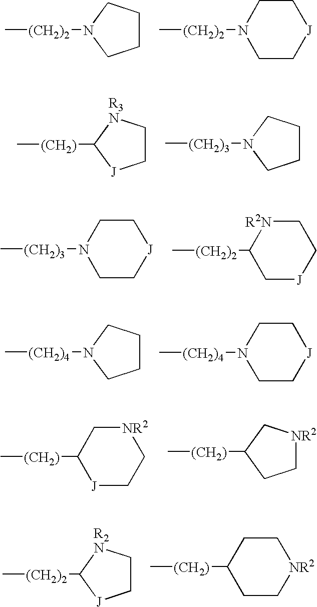

where each J independently represents CH₂, O, S(O)ₘ (wherein m=0-2), NR⁶, NC(O)R⁶, NC(O)OR⁶, NC(O)N(R⁶)₂, or NSO₂R⁶, where each R⁶ is H or a C₁₋₁₀ alkyl that can be substituted with one or more groups selected from halo, ═O, OR′, NR′₂, S(O)ₘR′, COOR′, and CONR′₂, where each R′ is H or C1-C4 alkyl optionally substituted with one or more halo or ═O, and m=0-2; and R² is as defined above. In certain embodiments, m is 2. In some embodiments, R⁶ is a C1-C4 alkyl that can be substituted with halo, ═O, OR′, NR′₂, S(O)ₘR′, S(O)ₘNR′₂, COOR′, or CONR′₂, where each R′ is H or C1-C4 alkyl optionally substituted with one or more halo or ═O, and m=0-2; in certain embodiments, m is 2.

In the above formulas (1), (2) and (3), B is absent when Z¹ is N. Similarly, when Z² is N, X is absent; when Z³ is N, A is absent; and when Z⁴ is N, V is absent.

In the above formulas (2) and (3), Z may be N or it may be CR³. When Z is CR³, it is CH or C-L-NR¹R² in certain embodiments.

In some embodiments, Z¹ is N, so B is absent; and Z² is a substituted carbon, so X is not H, while Z³ and Z⁴ are each carbons, and A and V are both H. In some embodiments, at least one of B, X, or A is halo and Z¹, Z², and Z³ are each C. In other embodiments, X and A are not both H when Z² and Z³ are C. In the above formulas (1), (2) and (3), V may be H in certain embodiments where Z⁴ is C.

In one embodiment, each of Z¹, Z², Z³ and Z⁴ is C. In another embodiment, three of Z¹, Z², Z³ and Z⁴ represent C, and the other represents N. For example, Z¹, Z² and Z³ are C, and Z⁴ is N. Alternatively, Z¹, Z² and Z⁴ are C, and Z³ is N. In other examples, Z¹, Z³ and Z⁴ are C and Z² is N. In yet other examples, Z², Z³ and Z⁴ are C, and Z¹ is N.

In another embodiment, two of Z¹, Z², Z³ and Z⁴ are C, and the other two are non-adjacent nitrogens. For example, Z¹ and Z³ may be C, while Z² and Z⁴ are N. Alternatively, Z¹ and Z³ may be N, while Z² and Z⁴ may be C. In other examples, Z¹ and Z⁴ are N, while Z² and Z³ are C.

In some embodiments, Z¹-Z⁴ are C and each of B, X, A, and V is H. In many embodiments, at least one of B, X, A, and V is H and the corresponding adjacent Z¹-Z⁴ atom is C. For example, any two of B, X, A, and V may be H. In one example, V and B may both be H. In another, B and A are both H, and X is not H. In other examples, any three of B, X, A, and V are H and the corresponding adjacent Z¹-Z⁴ atom is C.

In certain embodiments, one of B, X, A, and V is a halogen (e.g., fluorine) and the corresponding adjacent Z¹-Z⁴ is C. In other embodiments, two of X, A, and V are selected from halogen and SR², wherein R² is as defined above; and each corresponding adjacent Z²-Z⁴ is C. For example, each X and A may be a halogen.

In other examples, each X and A present may be SR², wherein R² is as defined above; in certain embodiments, R² is H or C₁₋₁₀ alkyl substituted with an aryl or heteroaryl group such as phenyl or pyrazine, which aryl or heteroaryl such as phenyl or pyrazine may itself be substituted. In yet other examples, any of B, V, A and X may be an alkynyl such as a propargyl group, a fluorinated alkyl such as CF₃, CH₂CF₃, perfluorinated C₂-C₁₀ alkyls, etc.; cyano, nitro, amides, sulfonyl amides, or carbonyl groups such as COR². Sometimes, at least one of V, A, B, and X is OR², where each R² is as defined above; in certain embodiments, R² is H or C₁₋₁₀ alkyl optionally substituted with an aryl or heteroaryl group such as phenyl or pyrazine, which aryl or heteroaryl group such as phenyl or pyrazine may itself be substituted.

In each of the above formulas, X, V, B, and A if present may independently be NR¹R², wherein R¹ and R² are as defined above, and wherein in any NR¹R² group, R¹ and R² can optionally cyclize to form an azacyclic group. In some embodiments, R¹ is H or C₁₋₁₀ alkyl, and R² is a C₁₋₁₀ alkyl optionally containing a heteroatom selected from N, O and S in place of one carbon atom, a C₃₋₆ cycloalkyl, aryl or a 5-14 membered heterocyclic ring containing one or more N, O or S.

If more than one NR¹R² moiety is present in a compound within the invention, as when both A and R³ comprise NR¹R² in a compound according to any one of the above formulas, each R¹ and each R² is independently selected. In one example, R² is a C₁₋₁₀ alkyl substituted with an optionally substituted 5-14 membered heterocyclic ring. For example, R² may be a C₁₋₁₀ alkyl substituted with morpholine, thiomorpholine, imidazole, aminodithiadazole, pyrrolidine, piperazine, pyridine or piperidine. Alternatively, R¹ and R² together with the N to which they are both bonded, may form an optionally substituted heterocyclic ring which may contain one or more additional N, O or S. For example, R¹ and R² together with N may form an azacyclic group selected from piperidine, pyrrolidine, piperazine, morpholine, thiomorpholine, imidazole, and aminodithiazole. Each of these azacyclic groups may be further substituted with, for example, one or more groups selected from halo, azido, ═O, CN, CF₃, CONR′₂, R′, CH₂R′, S(O)ₘR′, OR′C(═O)R′, C(═O)OR′, and NR′₂, where each R′ independently represents H or C₁-C₄ alkyl, and m=0-2, and where NR′₂ can additionally represent an azacyclic group such as piperidine, pyrrolidine, piperazine, morpholine, thiomorpholine, or imidazole, which azacyclic group can be substituted with one or more halo, azido, ═O, CN, CF₃, CONR″₂, R″, CH₂R″, S(O)ₘR″, OR″C(═O)R″, C(═O)OR″, and NR″₂, where each R″ independently represents H or C1-C4 alkyl, and each m is independently 0-2.

In certain embodiments, one of X, A B, and V is NR¹R². In such embodiments, NR¹R² sometimes represents an azacyclic group that may be substituted as described above. For example, X, A B, or V may be a 1-piperazinyl group, wherein the 4-position of the piperazine ring is substituted by C(═O)R″, C(═O)OR″, or by CONR″₂, where each R″independently represents H or C1-C4 alkyl. In certain such embodiments, Z¹ is N, Z² is C, and X is NR¹R₂, which represents an azacyclic group. In some such embodiments, A and V are present and each represent H, and in some such embodiments X represents a substituted piperazine, and the substituent at position 4 of the piperazine ring is an acyl group such as acetyl.

In one embodiment, the present invention provides compounds having formula (1), (2) or (3) as described above, wherein:

each of A, V and B if present is independently H or halogen (e.g., chloro or fluoro);

X is NR¹R², wherein R¹ and R² are as defined above and may be taken together to form an azacyclic group, or one of R¹ and R² may be taken together with at least a portion of L to form an optionally substituted heterocyclic, aryl or heteroaryl ring,

or X can represent an aryl or heteroaryl ring that may be substituted with halo, C1-C4 alkyl, C1-C4 haloalkyl, or C1-C4 alkoxy;

Z, if present, is N or CH;

W together with the two carbons of the imidazole/imidazoline ring to which it is fused forms a 5- or 6-membered ring that is further fused with an optionally substituted aryl or heteroaryl ring; and

-L-NR¹R² represents (CH₂)₂₋₄—NR¹R², wherein NR¹R² represents an azacyclic group selected from piperidine, pyrrolidine, piperazine, morpholine, thiomorpholine, and imidazole, each of which may be substituted with one or more groups selected from halo, azido, ═O, CN, CF₃, CONR′₂, R′, CH₂R′, S(O)ₘR′, OR′ C(═O)R′, C(═O)OR′, and NR′₂, where each R′ independently represents H or C1-C4 alkyl, and m=0-2, and where NR′ 2 can additionally represent an azacyclic group such as piperidine, pyrrolidine, piperazine, morpholine, thiomorpholine, or imidazole, which azacyclic group can be substituted with one or more halo, azido, ═O, CN, CF₃, CONR″₂, R″, CH₂R″, S(O)ₘR″, OR C(═O)R″, C(═O)OR″, and NR″₂, where each R″ independently represents H or C1-C4 alkyl, and each m is independently 0-2;

or -L-NR¹R² represents a group of formula (4):

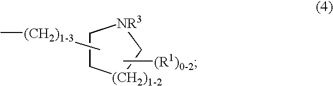

where R¹ and R³ are as defined above, and the optional substituents R¹ and the attachment point for the alkylene linker (CH₂)₁₋₃ can be at any position on the ring other than the nitrogen atom.

In another embodiment, the present invention provides compounds having formula (1), (2) or (3), wherein:

Z¹ is N, and Z², Z³ and Z⁴ are each C;

A and B are each independently H or halogen (e.g., chloro or fluoro);

X is H or an azacyclic group selected from piperidine, pyrrolidine, piperazine, morpholine, thiomorpholine, imidazole, which may be substituted with one or more substituents selected from halo, azido, ═O, CN, CF₃, CONR′₂, R′, CH₂R′, SR′, OR′C(═O)R′, and NR′₂, where each R′ independently represents H or C1-C4 alkyl or each NR′₂ can represent an optionally substituted azacyclic group;

Z, if present, is CH or N;

W together with the two carbon atoms of the imidazole/imidazoline ring to which it is fused forms an optionally substituted 5- or 6-membered ring that is further fused with an optionally substituted aryl or heteroaryl ring; and

-L-NR¹R² represents (CH₂)₂₋₄—NR¹R², wherein NR¹R² represents an azacyclic group selected from piperidine, pyrrolidine, piperazine, morpholine, thiomorpholine, and imidazole, each of which may be substituted with halo, azido, ═O, CN, CF₃, CONR′₂, R′, CH₂R′, S(O)ₘR′, OR′C(═O)R′, C(═O)OR′, and NR′₂, where each R′ independently represents H or C1-C4 alkyl, and m=0-2, and where NR′₂ can additionally represent an azacyclic group such as piperidine, pyrrolidine, piperazine, morpholine, thiomorpholine, or imidazole, which azacyclic group can be substituted with one or more halo, azido, ═O, CN, CF₃, CONR″₂, R″, CH₂R″, S(O)ₘR″, OR″ C(═O)R″, C(═O)OR″, and NR″₂, where each R″ independently represents H or C1-C4 alkyl, and each m is independently 0-2;

or -L-NR¹R² represents a group of formula (4):

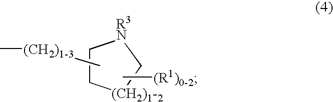

where R¹ and R³ are as defined above, and the optional substituents R¹ and the attachment point for the alkylene linker —(CH₂)₁₋₃— can be at any position on the ring other than the nitrogen atom.

In each of the above formula, unless otherwise indicated, each optionally substituted moiety may be substituted with one or more halo, OR², NR¹R², carbamate, C₁₋₁₀ alkyl, C₂₋₁₀ alkenyl, each of which is further optionally substituted by halo, ═O, aryl or one or more heteroatoms; inorganic substituents, aryl, carbocyclic or a heterocyclic ring. Other substituents include but are not limited to alkynyl, cycloalkyl, fluorinated alkyls such as CF₃, CH₂CF₃, perfluorinated alkyls, etc.; oxygenated fluorinated alkyls such as OCF₃ or OCH₂CF₃, etc.; cyano, nitro, COR², NR²COR², S(O)ₘ, COOR², CONR²₂, OCOR², OCOOR², OCONR²₂, NRCONR²₂, NRC(NR)(NR²₂), NR(CO)NR²₂, and SO₂NR²₂, wherein each R² is as defined in formula 1 and each m is independently 0-2.

Illustrative examples of optionally substituted heterocyclic rings include but are not limited to tetrahydrofuran, 1,3-dioxolane, 2,3-dihydrofuran, tetrahydropyran, benzofuran, isobenzofuran, 1,3-dihydro-isobenzofuran, isoxazole, 4,5-dihydroisoxazole, piperidine, pyrrolidine, pyrrolidin-2-one, pyrrole, pyridine, pyrimidine, octahydro-pyrrolo[3,4-b]pyridine, piperazine, pyrazine, morpholine, thiomorpholine, imidazole, aminodithiadazole, imidazolidine-2,4-dione, benzimidazole, 1,3-dihydrobenzimidazol-2-one, indole, thiazole, benzothiazole, thiadiazole, thiophene, tetrahydro-thiophene 1,1-dioxide, diazepine, triazole, diazabicyclo[2.2.1]heptane, 2,5-diazabicyclo[2.2.1]heptane, and 2,3,4,4a,9,9a-hexahydro-1H-β-carboline.

In certain embodiments, the invention includes a compound of formula (1) substituted as described for any of the preceding embodiments. In certain of these embodiments, the compound is a compound of formula (1a):

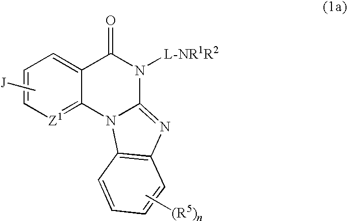

wherein Z¹ is N or CH or CX where X is fluoro or chloro;

each R⁵ independently represents a substitutent selected from halo, CN, CF₃, OR², NR¹R², S(O)ₘR², and C₁₋₆ alkyl and C₂₋₆ alkenyl, each optionally substituted by one or more substituents selected from halo, ═O, OR², S(O)ₘR², and NR¹R², where each m is independently 0-2, and R¹ and R² are as defined for formula (1);

J represents one or two substituents selected from halo, CN, CF₃, OR², NR¹R², S(O)ₘR², and C₁₋₆ alkyl and C₂₋₆ alkenyl, each optionally substituted by one or more substituents selected from halo, ═O, OR², S(O)ₘR², and NR¹R², where each m is independently 0-2, and R¹ and R² are as defined for formula (1);

n is 0-4;

L is (CH₂)₁₋₄; and

NR¹R² represents an azacyclic group.

In certain embodiments, the invention includes a compound of formula (2a):

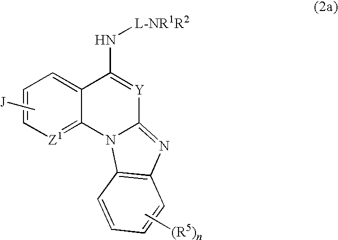

wherein Z¹ is N or CH or CX, where X is fluoro or chloro;

each R⁵ independently represents a substitutent selected from halo, CN, CF₃, OR², NR¹R², S(O)ₘR², and C₁₋₆ alkyl and C₂₋₆ alkenyl, each optionally substituted by one or more substituents selected from halo, ═O, OR², S(O)ₘR², and NR¹R², where each m is independently 0-2, and R¹ and R² are as defined for formula (1);

Y is N or CH or CX′, where X′ is an optionally substituted group selected from C₁₋₆ alkyl, C₆₋₁₀ aryl, and C₅₋₁₂ heteroaryl, or X is selected from halo, nitro, OR′, SR′, SO₂R′, NR′₂, CN, CF₃, COOR′, and CONR′₂;

J represents one or two substituents selected from halo, CN, CF₃, OR², NR¹R², S(O)ₘR², and C₁₋₆ alkyl and C₂₋₆ alkenyl, each optionally substituted by one or more substituents selected from halo, ═O, OR², S(O)ₘR², and NR¹R², where each m is independently 0-2, and R¹ and R² are as defined for formula (1);

n is 0-4; and

L is (CH₂)₁—4; and NR¹R² represents an azacyclic group; or

-L-NR¹R² represents a group of formula (4):

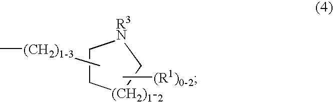

where R¹ and R³ are as defined above, and the optional substituents R¹ and the attachment point for the alkylene linker —(CH₂)₁₋₃— can be at any position on the ring other than the nitrogen atom.

In certain embodiments, the invention includes a compound of formula (2a):

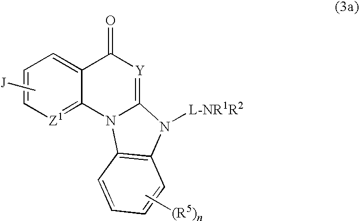

wherein Z¹ is N or CH or CX, where X is fluoro or chloro;

each R⁵ independently represents a substitutent selected from halo, CN, CF₃, OR², NR¹R², S(O)ₘR², and C₁₋₆ alkyl and C₂₋₆ alkenyl, each optionally substituted by one or more substituents selected from halo, ═O, OR², S(O)ₘR², and NR¹R², where each m is independently 0-2, and R¹ and R² are as defined for formula (1);

Y is N or CH or CX′, where X′ is an optionally substituted group selected from C₁₋₆ alkyl, C₆₋₁₀ aryl, and C₅₋₁₂ heteroaryl group, or X′ is selected from halo, nitro, OR′, SR′, SO₂R′, NR′₂, CN, CF₃, COOR′, and CONR′₂;

J represents one or two substituents selected from halo, CN, CF₃, OR², NR¹R², S(O)ₘR², and C₁₋₆ alkyl and C₂₋₆ alkenyl, each optionally substituted by one or more substituents selected from halo, ═O, OR², S(O)ₘR², and NR¹R², where each m is independently 0-2, and R¹ and R² are as defined for formula (1);

n is 0-4; and

L is (CH₂)₁₋₄ and NR¹R² represents an azacyclic group; or

-L-NR¹R² represents a group of formula (4):

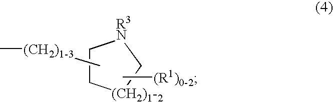

where R¹ and R³ are as defined above, and the optional substituents R¹ and the attachment point for the alkylene linker —(CH₂)₁₋₃— can be at any position on the ring other than the nitrogen atom.

In compounds of formula (1a), (2a) or (3a), Z¹ is sometimes N, and in certain embodiments J represents 1-2 halo substituents or J represents an azacyclic group that may be attached at the ring carbon adjacent to Z¹.

In compounds of formula (1a), n is sometimes 0-2, and L is sometimes (CH₂)₂ or (CH₂)₃.

In compounds of formula (2a), n is sometimes 0-2, and L is sometimes (CH₂)₂ or (CH₂)₃.

In compounds of formula (3a), n is sometimes 0-2, and L is sometimes (CH₂)₂ or (CH₂)₃.

The present invention also provides pharmaceutical compositions comprising a compound having any one of the above formulas and a pharmaceutically acceptable excipient. In one example, the composition comprises a compound having any one of the above formulas, polyethylene glycol, and propylene glycol in a buffer solution, or a compound of any of the above formulas in phosphate buffered saline (PBS), or acidified PBS.

Furthermore, the present invention relates to methods for reducing cell proliferation and/or inducing cell death, comprising contacting a system with an effective amount of a compound having any one of the above formula, or a pharmaceutical composition thereof and optionally in combination with a chemotherapeutic agent, thereby reducing cell proliferation and/or inducing cell death, such as apoptosis or apoptotic cell death, in said system. The system may be a cell or a tissue. In one embodiment, the system includes a pancreatic cell, such as a cell from a subject or a cultured cell (e.g., in vitro or ex vivo). In particular embodiments, the system includes a pancreatic cancer cell. In one embodiment, the system is a cell line such as PC3, HCT116, HT29, MIA Paca-2, HPAC, Hs700T, Panc10.05, Panc 02.13, PL45, SW 190, Hs 766T, CFPAC-1 and PANC-1.

The present invention also provides methods for ameliorating a cell proliferative disorder, comprising administering to a subject in need thereof an effective amount of a compound having any one of the above formulas, or a pharmaceutical composition thereof, and optionally in combination with a chemotherapeutic agent, thereby ameliorating said cell-proliferative disorder. For example, cell proliferation may be reduced, and/or cell death, such as apoptosis or apoptotic cell death, may be induced. The cell proliferative disorder may be a tumor or a cancer in a human or animal subject. In a particular embodiment, the cancer is pancreatic cancer, including non-endocrine and endocrine tumors. Illustrative examples of non-endocrine tumors include but are not limited to adenocarcinomas, acinar cell carcinomas, adenosquamous carcinomas, giant cell tumors, intraductal papillary mucinous neoplasms, mucinous cystadenocarcinomas, pancreatoblastomas, serous cystadenomas, solid and pseudopapillary tumors. An endocrine tumor may be an islet cell tumor.

The above methods for reducing cell proliferation and/or inducing cell death may also be practiced in combination with a procedure and/or a chemotherapeutic agent. Examples of procedures that may be used in combination with the methods of the present invention include but are not limited to radiotherapy or surgery. In certain embodiments, the compounds of the present invention are administered in combination with gemcitabine, and used to reduce cell proliferation, induce cell death, and/or ameliorate a cell proliferative disorder.

Furthermore, the present invention provides methods for reducing microbial titers, comprising contacting a system with an effective amount of a compound having any one of the above formula, or a pharmaceutical composition thereof and optionally with an antimicrobial agent, thereby reducing microbial titers. The system may be a cell or a tissue. The present invention also provides methods for ameliorating a microbial infection, comprising administering to a subject in need thereof an effective amount of a compound having any one of the above formula, or a pharmaceutical composition thereof and optionally with an antimicrobial agent, thereby ameliorating said microbial infection. The subject may be human or an animal. The microbial titers may be viral, bacterial or fungal titers.

The present invention also relates to methods for determining interaction selectivity between a compound having any one of the above formula, and nucleic acids capable of forming a quadruplex structure, comprising: a) contacting a compound in the absence of a competitor molecule with three or more nucleic acids capable of forming a quadruplex structure, wherein each nucleic acid is not a telomere nucleic acid; b) measuring a direct interaction between the compound and said three or more nucleic acids; and c) determining interaction selectivity from a comparison of the interaction measurements. In one example, three or more nucleic acids comprise a nucleotide sequence located 5′ of an oncogene nucleotide sequence. The oncogene may be MYC, HIF, VEGF, ABL, TGF, PDGFα, MYB, SPARC, HER, VAV, RET, H-RAS, EGF, SRC, BCL-1, BCL-2, DHFR, or HMGA. In determining interaction selectivity, the compound may be separately contacted with each of said three or more nucleic acids in a different vessel. Furthermore, the interaction selectivity may be determined from a comparison of IC₅₀ values.

The compounds of the present invention may or may not interact with regions of DNA that can form quadruplexes. In certain embodiments, the compounds of the present invention may bind and/or stabilize a propeller quadruplex. Examples of propeller quadruplexes include but are not limited to H-RAS, RET, BCL-1, DHFR, TGF-β, HIF-1α, VEGF, c-Myc, or PDGFα. In another embodiment, the compound may bind and/or stabilize a propeller or a basket quadruplex. For example, the compound may bind and/or stabilize BCL-2.

The present invention also provides methods for inducing cell death, such as apoptotic cell death (apoptosis), comprising administering to a system or a subject in need thereof an effective amount of a compound having any one of the above formula, or a pharmaceutical composition thereof and optionally with a chemotherapeutic agent. The subject may be a human or an animal, and the system may be a cell or a tissue. Thus in one embodiment, the invention provides a composition comprising a cell and a compound according to one of the above formulas, which composition is formed by exposing a cell to a compound of formula (1), (2), or (3). Such compositions are useful in predicting the effect of the compound or compositions comprising such compound on a tissue or subject, and for providing a cell or tissue with an improved safety profile (reduced probability of pathogenic effects) when the cell is to be administered or otherwise exposed to a living subject, as when the cell is part of a tissue for implant or transplant into a subject.

The present invention also provides methods for treating or ameliorating a disorder mediated by oncogene overexpression, such as c-Myc overexpression, comprising administering to a system or a subject in need thereof an effective amount of a compound having any of the formula, or a pharmaceutical composition thereof and optionally with a chemotherapeutic agent. The subject may be human or an animal, and the system may be a cell or a tissue.

Compounds of the above formulas are also capable of modulating the activities of various protein kinases, as they contain structural features that are known to bind to protein kinases, and are accordingly useful for the identification of protein kinase modulators using screening methods that are well known in the art. Representative screening methods for certain kinases are provided herein. Accordingly, the invention provides a method for identifying a modulator of a protein kinase, which modulator sometimes is a potent modulator of one or more particular protein kinases. This method comprises screening a library of compounds of formula (1), (2) or (3), which library contains at least 10 different compounds each of which is of formula (1), (2) or (3), and preferably at least 100 of such compounds, for their ability to modulate the activity of a protein kinase. Alternatively, the method comprises screening a set of protein kinases, such as at least three or at least ten protein kinases, with a compound of formula (1), to determine a differential activity profile. These methods allow the user to identify a compound of formula (1) having a desired level of activity and/or selectivity as a protein kinase activity modulator, which compound may be used to initiate a drug development program.

Thus in another embodiment, the invention provides a library of compounds, which library comprises at least 10 compounds having a formula selected from (1), formula (2) and formula (3). The library preferably contains at least 100 such compounds. This library can be used to identify compounds having one or more of the activities described herein, or a specific combination of such activities using methods known in the art. The method is particularly useful for identifying molecules having a threshold level of activity for binding to quadruplex DNA or inhibiting formation of quadruplex DNA, or having a threshold level of activity against a specific protein kinase or set of protein kinases; or molecules having a threshold level of activity as a modulator of binding of a nucleic acid to a protein such as nucleolin.

Thus in another embodiment, the invention provides a composition comprising an isolated protein kinase complexed with a compound of formula (1), (2), or (3). Such complexes are useful for the information they provide about the binding site of a modulating compound to the particular kinase, and as a research tool for analyzing the structure of the kinase. Such complexes are also useful because they may be more readily crystallized than the uncomplexed kinase, allowing crystallization and crystal structure determination where it would not be possible without the bound modulating compound.

Also provided herein is a method for identifying a molecule that modulates an interaction between a ribosomal nucleic acid and a protein that interacts with the nucleic acid, which comprises: (a) contacting a nucleic acid containing a human ribosomal nucleotide sequence and the protein with a test molecule having any of the structures disclosed above, where the nucleic acid is capable of binding to the protein, and (b) detecting the amount of the nucleic acid bound or not bound to the protein, whereby the test molecule is identified as a molecule that modulates the interaction when a different amount of the nucleic acid binds to the protein in the presence of the test molecule than in the absence of the test molecule. In some embodiments, the protein is selected from the group consisting of Nucleolin, Fibrillarin, RecQ, QPN1 and functional fragments of the foregoing.

In some embodiments, provided is a method for identifying a molecule that causes nucleolin displacement, which comprises (a) contacting a nucleic acid containing a human ribosomal nucleotide sequence and a nucleolin protein with a test molecule, where the nucleic acid is capable of binding to the nucleolin protein, and (b) detecting the amount of the nucleic acid bound or not bound to the nucleolin protein, whereby the test molecule is identified as a molecule that causes nucleolin displacement when less of the nucleic acid binds to the nucleolin protein in the presence of the test molecule than in the absence of the test molecule. In some embodiments, the nucleolin protein is in association with a detectable label, and the nucleolin protein sometimes is in association with a solid phase. The nucleic acid sometimes is in association with a detectable label, and the nucleic acid may be in association with a solid phase in certain embodiments. The nucleic acid may be DNA, RNA or an analog thereof, and may comprise a nucleotide sequence described above in specific embodiments. In some embodiments the test molecule is an analog described herein, such as a compound of formula (1), (2) or (3). Provided also is a composition comprising a nucleic acid having a ribosomal nucleotide sequence provided herein, or substantially identical sequence thereof, and a protein that binds to the nucleotide sequence (e.g., Nucleolin, Fibrillarin, RecQ, QPN1 and functional fragments of the foregoing).

Also provided is a method for identifying a molecule that binds to a nucleic acid containing a human ribosomal nucleotide sequence, which comprises: (a) contacting a nucleic acid containing a human ribosomal nucleotide sequence described herein, a compound that binds to the nucleic acid and a test molecule, and (b) detecting the amount of the compound bound or not bound to the nucleic acid, whereby the test molecule is identified as a molecule that binds to the nucleic acid when less of the compound binds to the nucleic acid in the presence of the test molecule than in the absence of the test molecule. The compound sometimes is in association with a detectable label, and at times is radiolabled. In certain embodiments, the compound is a quinolone-type compound, (e.g., an analog described herein, such as a compound of formula (1), (2), or (3)) or a porphyrin. The nucleic acid may be in association with a solid phase in certain embodiments. The nucleic acid may be DNA, RNA or an analog thereof, and may comprise a nucleotide sequence described above in specific embodiments. The nucleic acid may form a quadruplex, such as an intramolecular quadruplex, in certain embodiments. Examples of ribosomal nucleotide sequences are described herein and in co-pending provisional patent application Ser. No. 60/789,109, filed Apr. 3, 2006, and entitled HUMAN RIBOSOMAL DNA (rDNA) AND RIBOSOMAL RNA (rRNA) QUADRUPLEX NUCLEIC ACIDS AND USES THEREOF.

Also provided herein is a method for identifying a modulator of nucleic acid synthesis, which comprises contacting a template nucleic acid, a primer oligonucleotide having a nucleotide sequence complementary to a template nucleic acid nucleotide sequence, extension nucleotides, a polymerase and a test molecule, under conditions that allow the primer oligonucleotide to hybridize to the template nucleic acid, wherein the template nucleic acid comprises a human ribosomal nucleotide sequence, and detecting the presence, absence or amount of an elongated primer product synthesized by extension of the primer nucleic acid, whereby the test molecule is identified as a modulator of nucleic acid synthesis when less of the elongated primer product is synthesized in the presence of the test molecule than in the absence of the test molecule. In certain embodiments, the method is directed to identifying a modulator of RNA synthesis, and in certain embodiments, identifying a modulator of nucleolar RNA synthesis. The template nucleic acid sometimes is DNA and at times is RNA, and the template can include by way of example any one or more of the ribosomal nucleotide sequences described herein. The polymerase sometimes is a DNA polymerase and at times is a RNA polymerase.

In a specific assay embodiment, provided herein is a method for identifying a molecule that modulates ribosomal RNA (rRNA) synthesis, which comprises: contacting cells with a test molecule, contacting a ribosomal nucleotide sequence with one or more primers that amplify a portion thereof and a labeled probe that hybridizes to the amplification product, and detecting the amount of the amplification product by hybridization of the labeled probe, whereby a test molecule that reduces or increases the amount of amplification product is identified as a molecule that modulates rRNA synthesis. The labeled probe in some embodiments is added after the primers are added and the rRNA is amplified, and in certain embodiments, the labeled probe and the primers are added at the same time. The portion of ribosomal nucleotide sequence amplified sometimes is at the 5′ end of rDNA. In certain embodiments, the test molecule is a compound of formula (1), (2) or (3) as described herein.

In another aspect, the present invention provides methods for preparing compounds having formula (1), (2), or (3) as set forth herein.

In the above methods, the purity of the isolated compounds may be between 90 and 99%. For example, the isolated compounds may have a purity between 90 and 95%.

### DEFINITIONS

As used herein, the terms “alkyl,” “alkenyl” and “alkynyl” include straight-chain, branched-chain and cyclic monovalent hydrocarbyl radicals, and combinations of these, which contain only C and H when they are unsubstituted. Examples include methyl, ethyl, isobutyl, cyclohexyl, cyclopentylethyl, 2-propenyl, 3-butynyl, and the like. The total number of carbon atoms in each such group is sometimes described herein, e.g., when the group can contain up to ten carbon atoms it can be represented as 1-10C or as C1-C10 or C1-10.

Where an alkyl, alkenyl or alkynyl group is described as ‘optionally substituted’, it can be unsubstituted or it can be substituted with one or more substituents including but not limited to OR¹, amino, amido, halo, ═O, aryl, heterocyclic groups, or inorganic substituents. The number of substituents allowed is limited by the number of available valences on the alkyl, alkenyl or alkynyl group. As used herein, ═O represents a carbonyl oxygen, which can be a substituent where a divalent group is permissible.

Alkyl groups may also include an unsaturated bond, but they are typically connected to the molecule through a saturated carbon of the alkyl group. Alkenyl and alkynyl groups may contain more than one unsaturation, and may contain a mixture of double and triple bonds.

Alkyl groups can also include one or more heteroatoms selected from N, O and S in place of carbon atoms comprising the alkyl group. Alkyl groups are connected via a carbon atom of the alkyl group to the remainder of the molecule, and no more than two contiguous carbon atoms can be replaced by heteroatoms. Where N is present in such groups, it is understood that it is trivalent and must be suitably substituted according to commonly understood principles of chemical stability.

As used herein, ‘alkylene’ takes its ordinary meaning, and refers to a non-aromatic hydrocarbon group having two open valences (divalent), and thus requiring two additional groups to define a stable compound. Alkylene groups can include straight chains, branched chains or rings, or a combination of these. Typical examples include methylene, (CH₂)₂₋₆, CH(CH₃), CMe₂, cyclopropan-1,1-diyl, and cyclohexan-1,4-diyl, for example. Alkylene groups can be substituted with groups suitable as substituents for alkyl groups as further set forth herein, so —C(O)— could also be an alkylene, for example.

As used herein, the term “carbocycle” refers to a cyclic compound containing only carbon atoms in the ring, whereas a “heterocycle” refers to a cyclic compound comprising at least one heteroatom selected from N, O and S as a ring member. The carbocyclic and heterocyclic structures encompass compounds having monocyclic, bicyclic or multiple ring systems. ‘Carbocyclic’ groups may contain 3-10 ring atoms, commonly 3-8 or 5-6 ring atoms; and ‘heterocyclic’ groups may contain 3-14 ring atoms, commonly 3-10 ring atoms, and more commonly 5-8 ring atoms.

The term ‘halo’ as used herein refers to any of the halogens, typically including F, Cl, Br and I. More commonly, halo when used as a substituent, refers to either F or Cl or a mixture thereof.

As used herein, the term “heteroatom” refers to any atom that is not carbon or hydrogen, such as nitrogen, oxygen or sulfur, for example. Where it is used to describe an atom of a ring or chain, it can refer to O, S, N, P, or Si, each of which is further substituted in accordance with commonly understood limitations of chemical stability to provide a compound that is at least relatively stable in an aqueous medium. Thus, for example, N is trivalent and will be substituted accordingly; S may be divalent as in a thioether, trivalent as in a sulfoxide, or hexavalent as in a sulfonyl group. Typically, heteroatoms that are included in an alkyl group or a ring are selected from N, O and S.

As used herein, the term “aryl” refers to a polyunsaturated, typically aromatic hydrocarbon substituent containing at least one aromatic ring that does not have a heteroatom as a ring member, whereas “heteroaryl” or “heteroaromatic” refers to an aromatic group containing at least one heteroatom as a ring member. The aryl and heteroaryl structures encompass compounds having monocyclic, bicyclic or multiple ring systems, and thus they may include a mixture of aryl and heteroaryl groups provided that where the group is referred to as ‘aryl’ it is attached to the molecule at a position of an aryl ring of the ‘aryl’ group, and where it is described as ‘heteroaryl’ it is attached to the molecule at a position of a heteroaryl ring of the group.

These groups may be single (isolated) rings or they may be ring systems including multiple fused rings. Aryl groups typically include phenyl and naphthyl, and may include additional rings; thus aryl groups can include an indole, benzofuran or tetrahydronaphthyl group, for example, provided that the point of attachment is on a ring that is an aryl ring.

Heteroaryl groups include at least one heteroatom as a ring member; more than one heteroatom may be present as ring members in a heteroaryl group, provided that not more than two contiguous ring atoms are heteroatoms. The point of attachment for a heteroaryl group can be either carbon or a heteroatom where the valence permits; for example, indole would be a heteroaryl group if linked to the molecule through any of the atoms of the five-membered ring (positions 1-3 of indole). However, an indole can be an aryl group if the point of attachment of the indole to the molecule is on the phenyl ring of the indole, i.e. ring positions 4-7 of the indole.

As used herein, the term ‘azacyclic’ refers to a heterocyclic group containing at least one nitrogen atom as a ring member. In certain embodiments, the azacyclic group is not aromatic. The azacyclic group may further contain one or two additional heteroatoms as ring members, which are selected from N, O, and S(O)ₘ, where m is 0-2. It may be substituted with suitable substituents for an alkyl group, for example it may be optionally substituted with halo, ═O, R, OR, NR₂, SR, CN, COOR, CONR₂, COR, CONR₂, etc. Typically the azacyclic group is linked to the structure of formula (1), (2) or (3) through the N of the azacyclic ring. Specific examples include pyrrolidinyl, pyrrolidinonyl, morpholinyl, thiomorpholinyl, azetidinyl, piperidinyl and piperazinyl groups, including N4-substituted piperazine groups such as N4-methyl-piperazin-1-yl and N4-acetyl-piperazin-1-yl. In many embodiments, the atom in the azacyclic ring through which the azacyclic group is attached to another portion of a molecule is N; thus, for example, in certain embodiments the azacyclic group is a 1-pyrrolidinyl or a 1-piperazinyl group, each of which may be further substituted as described above.

‘Heterocycles’ can include saturated, unsaturated and aromatic ring systems that include at least one heteroatom as a ring member. Illustrative examples of heterocycles include but are not limited to furan, tetrahydrofuran, 1,3-dioxolane, 2,3-dihydrofuran, pyran, tetrahydropyran, benzofuran, isobenzofuran, 1,3-dihydro-isobenzofuran, isoxazole, 4,5-dihydroisoxazole, piperidine, pyrrolidine, pyrrolidin-2-one, pyrrole, pyridine, pyrimidine, octahydro-pyrrolo[3,4-b]pyridine, piperazine, pyrazine, morpholine, thiomorpholine, imidazole, imidazolidine-2,4-dione, 1,3-dihydrobenzimidazol-2-one, indole, thiazole, benzothiazole, thiadiazole, thiophene, tetrahydro-thiophene 1,1-dioxide, diazepine, triazole, guanidine, diazabicyclo[2.2.1]heptane, 2,5-diazabicyclo[2.2.1]heptane, 2,3,4,4a,9,9a-hexahydro-1H-β-carboline, oxirane, oxetane, dioxane, lactones, aziridine, azetidine, piperidine, lactams, and may also encompass heteroaryls. Other illustrative examples of heteroaryls include but are not limited to furan, pyrrole, pyridine, pyrimidine, imidazole, benzimidazole and triazole.

As used herein, the term “inorganic substituent” refers to substituents that do not contain carbon or substituents, other than common ones like —OR and —SR, that can contain carbon but are linked to the molecule through a non-carbon atom. Examples of inorganic substituents include but are not limited to nitro, halogen, azido, and groups such as sulfonates, sulfinates, phosphates, and phosphonates, as either their acid forms or as simple C₁-C₄ esters, e.g., a dimethyl phosphonate.

Unless otherwise described, an alkyl, alkenyl, alkynyl, cycloalkyl, or alkylene, or other non-aryl hydrocarbon group can be substituted by one or more suitable groups. Typical substituents include, but are not limited to, halo, ═O, ═N—CN, ═N—OR, ═NR, OR, NR₂, SR, SO₂R, SO₂NR₂, NRSO₂R, NRCONR₂, NRCOOR, NRCOR, CN, COOR, CONR₂, OOCR, COR, and NO₂, wherein each R is independently H, C1-C8 alkyl, C2-C8 heteroalkyl, C1-C8 acyl, C2-C8 heteroacyl, C2-C8 alkenyl, C2-C8 heteroalkenyl, C2-C8 alkynyl, C2-C8 heteroalkynyl, C6-C10 aryl, or C5-C10 heteroaryl, and each R is optionally substituted with halo, ═O, ═N—CN, ═N—OR′, ═NR′, OR′, NR′₂, SR′, SO₂R′, SO₂NR′₂, NR′SO₂R′, NR′CONR′₂, NR′COOR′, NR′COR′, CN, COOR′, CONR′₂, OOCR′, COR′, and NO₂, wherein each R′ is independently H, C1-C8 alkyl, C2-C8 heteroalkyl, C1-C8 acyl, C2-C8 heteroacyl, C6-C10 aryl or C5-C10 heteroaryl. Alkyl, alkenyl and alkynyl groups can also be substituted by C1-C8 acyl, C2-C8 heteroacyl, C6-C10 aryl or C5-C10 heteroaryl, each of which can be substituted by the substituents that are appropriate for the particular group.

Unless otherwise described, each aryl and heteroaryl group can be substituted by one or more suitable groups. Suitable groups include optionally substituted C1-C8 alkyl, C2-C8 heteroalkyl, C2-C8 alkenyl, C2-C8 heteroalkenyl, C2-C8 alkynyl, C2-C8 heteroalkynyl, C1-C8 acyl, C2-C8 heteroacyl, C6-C10 aryl, C5-C12 heteroaryl, C7-C12 arylalkyl, or C6-C12 heteroarylalkyl group, or it can be halo, OR, NR₂, NROR, NRNR₂, S(O)ₘR, SO₂NR₂, NRSO₂R, NRCONR₂, NRCOOR, NRCOR, CN, COOR, CONR₂, OOCR, COR, or NO₂,

wherein each m is 0-2;

each R is independently H or C1-C8 alkyl, C2-C8 heteroalkyl, C2-C8 alkenyl, C2-C8 heteroalkenyl, C2-C8 alkynyl, C2-C8 heteroalkynyl, C1-C8 acyl, C2-C8 heteroacyl, C6-C10 aryl, C5-C10 heteroaryl, C7-C12 arylalkyl, or C6-C12 heteroarylalkyl,

and wherein two R can be linked to form a 3-8 membered ring, optionally containing one or more N, O or S;

and wherein each R group, and each ring formed by linking two R groups together, is optionally substituted with one or more substituents selected from halo, ═O, ═N—CN, ═N—OR′, ═NR′, OR′, NR′₂, SR′, SO₂R′, SO₂NR′₂, NR′SO₂R′, NR′CONR′₂, NR′COOR′, NR′COR′, CN, COOR′, CONR′₂, OOCR′, COR′, and NO₂,
wherein each R′ is independently H, C1-C6 alkyl, C2-C6 heteroalkyl, C1-C6 acyl, C2-C6 heteroacyl, C6-C10 aryl, C5-C10 heteroaryl, C7-12 arylalkyl, or C6-12 heteroarylalkyl, each of which is optionally substituted with one or more groups selected from halo, C1-C4 alkyl, C1-C4 heteroalkyl, C1-C6 acyl, C1-C6 heteroacyl, hydroxy, amino, and ═O;
and wherein two R′ can be linked to form a 3-7 membered ring optionally containing up to three heteroatoms selected from N, O and S.

In many compounds within the present invention, isomers including double bond isomers, restricted rotation isomers, optical isomers, and mixtures of these are possible. The invention includes each individual isomer as well as mixtures of various isomeric forms, and specifically includes racemic mixtures as well as individual enantiomers where a single chiral carbon is present. Where multiple chiral carbons are present, each individual diastereomer is included as well as mixtures that comprise a racemic mixture of one or more diastereomer. Many of the compounds herein can exist in different tautomeric forms, and it is understood that each tautomer is included within the scope of the invention as well.

The terms “treat,” “treatment” and “therapeutic effect” as used herein refer to reducing or stopping a cell proliferation rate (e.g., slowing or halting tumor growth) or reducing the number of proliferating cancer cells (e.g., removing part or all of a tumor). These terms also are applicable to reducing a titre of a microorganism in a system (i.e., cell, tissue, or subject) infected with a microorganism, reducing the rate of microbial propagation, reducing the number of symptoms or severity of symptoms, or reducing an effect of a symptom associated with the microbial infection, and/or removing detectable amounts of the microbe from the system. Examples of microorganism include but are not limited to virus, bacterium and fungus.

As used herein, the term “chemotherapeutic agent” refers to a therapeutic agent that may be used for treating or ameliorating a cell proliferative disorder such as tumors or cancer. Examples of chemotherapeutic agents include but are not limited to an antineoplastic agent, an alkylating agent, a plant alkaloid, an antimicrobial agent, a sulfonamide, an antiviral agent, a platinum agent, and other anticancer agents known in the art. Particular examples of chemotherapeutic agents include but are not limited to cisplatin, carboplatin, busulphan, methotrexate, daunorubicin, doxorubicin, cyclophosphamide, mephalan, vincristine, vinblastine, chlorambucil, paclitaxel, gemcitabine, and others known in the art. (See e.g., Goodman & Gilman's, The Pharmacological Basis of Therapeutics (9th Ed) (Goodman, et al., eds.) (McGraw-Hill) (1996); and 1999 Physician's Desk Reference (1998)).

As used herein, the term “apoptosis” refers to an intrinsic cell self-destruction or suicide program. In response to a triggering stimulus, cells undergo a cascade of events including cell shrinkage, blebbing of cell membranes and chromatic condensation and fragmentation. These events culminate in cell conversion to clusters of membrane-bound particles (apoptotic bodies), which are thereafter engulfed by macrophages.

### DESCRIPTION OF THE INVENTION

The present invention relates to tetracyclic imidazole or imidazoline compounds having formula (1), (2), or (3), or formula (1a), (2a), or (3a) and pharmaceutically acceptable salts, esters, and prodrugs thereof. The present invention also relates to methods for using the compounds described herein, such as in screening and in treatment and in the preparation of a medicament for treating conditions described herein. The compounds of the present invention may or may not interact with regions of DNA that can form quadruplexes.

The compounds of present invention having formula (1), (2), and (3) are reproduced below:

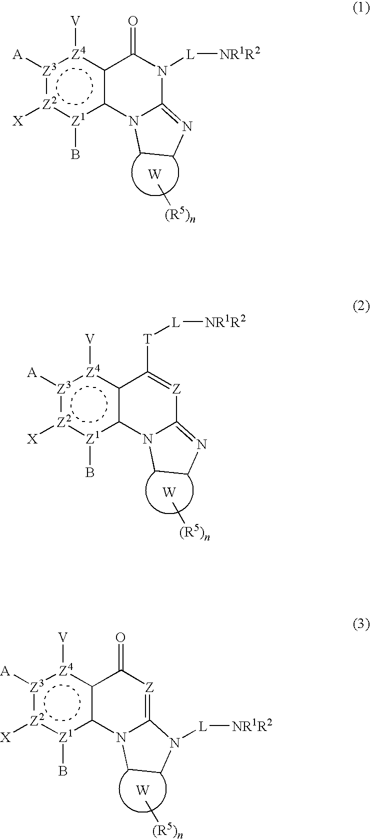

wherein each A, V, B, and X that is present is independently selected from H, halo, azido, CN, CF₃, CONR¹R², R², CH₂R², SR², C(═O)R², and NR¹R², wherein in NR¹R², R¹ and R² can optionally cyclize to form an optionally substituted azacyclic group; or

wherein A and X, or A and V, or X and B may form a carbocyclic ring, heterocyclic ring, aryl or heteroaryl ring, each of which may be optionally substituted with one or two R³ groups and/or may be fused with an additional ring;

wherein in L-NR¹R², R¹ and R² taken together may form an optionally substituted azacyclic group, or R¹ or R² taken together with at least a portion of L may form an optionally substituted heterocyclic ring;

each Z is independently CH, CR³ or N;

each Z¹, Z², Z³, and Z⁴ is independently C or N, provided no two of them represent adjacent nitrogen atoms;

T is O, S(O)ₘ or NR⁴;

each R¹ is H or a C₁₋₁₀ alkyl or C₂₋₁₀ alkenyl group that can be substituted with one or more substituents selected from halo, ═O, OR², NR²₂, S(O)ₘR², COOR², and CONR²₂;

each m is independently 0-2;

each n is independently 0-4;

each R² is independently H or an optionally substituted C₁₋₁₀ alkyl or optionally substituted C₂₋₁₀ alkenyl optionally containing one or more non-adjacent heteroatoms selected from N, O, and S in place of carbon atoms, and optionally including a carbocyclic or heterocyclic ring; or R² is an optionally substituted carbocyclic, heterocyclic, 6-10 membered aryl or 5-14 membered heteroaryl ring containing one or more N, O or S;

each R³ is independently an optionally substituted group selected from C₁₋₆ alkyl, C₆₋₁₀ aryl, and C₅₋₁₂ heteroaryl, or R³ is selected from halo, nitro, OR′, SR′, SO₂R′, NR′₂, CN, CF₃, COOR′, and CONR′₂, wherein each R′ is independently H or C₁₋₆ alkyl and can optionally include one N, O or S in place of a carbon atom, or R³ can be L-NR¹R² or CON(R′)-L-NR¹R², wherein in NR¹R², R¹ and R² can optionally cyclize to form an optionally substituted azacyclic group;

each R⁴ is H or a C₁₋₁₀ alkyl or C₂₋₁₀ alkenyl group that can be substituted with one or more substituents selected from halo, ═O, OR², NR²₂, S(O)ₘR², COOR², and CONR²₂;

each L is a divalent hydrocarbon linker having up to five atoms counted along the shortest path between the two open valence, which linker may include one or two heteroatoms and may be substituted by one or more groups selected from halo, ═O, C₁₋₆ alkyl, OR′, SR′, SO₂R′, NR′₂, CN, CF₃, COOR′, and CONR′₂, wherein each R′ is independently H or C₁₋₆ alkyl;

each W represents an optionally substituted aryl or heteroaryl ring, which may be a monocyclic group with 5-6 ring atoms, or may be a 5-6 membered ring that is fused with or bonded to one or more additional aryl, heterocyclic, or heteroaryl rings; and

each R⁵ is a substituent at any position on W, and is selected from H, halo, CN, CF₃, OR², NR¹R², and C₁₋₆ alkyl and C₂₋₆ alkenyl, each optionally substituted by one or more substituents selected from halo, ═O, OR², S(O)ₘR², and NR¹R², wherein in NR¹R², R¹ and R² can optionally cyclize to form an optionally substituted azacyclic group; or R⁵ can be an inorganic substituent; or two adjacent R⁵ may be linked to form a 5-6 membered substituted or unsubstituted carbocyclic or heterocyclic ring, optionally fused to an additional substituted or unsubstituted carbocyclic or heterocyclic ring;

or a pharmaceutically acceptable salt thereof.

The compounds of the present invention may be chiral. As used herein, a chiral compound is a compound that is different from its mirror image, and has an enantiomer. Furthermore, the compounds may be racemic, or an isolated enantiomer or stereoisomer. Methods of synthesizing chiral compounds and resolving a racemic mixture of enantiomers are well known to those skilled in the art. See, e.g., March, “Advanced Organic Chemistry,” John Wiley and Sons, Inc., New York, (1985), which is incorporated herein by reference.

Synthetic procedures for preparing the compounds of the present invention are illustrated in Schemes I-V, and in the Examples. Other variations in the synthetic procedures known to those with ordinary skill in the art may also be used to prepare the compounds of the present invention. The compounds are then isolated and purified by conventional methods.

The compounds also may be made as or converted into salts, and in certain embodiments they are made and used as pharmaceutically acceptable salts such as those described herein.

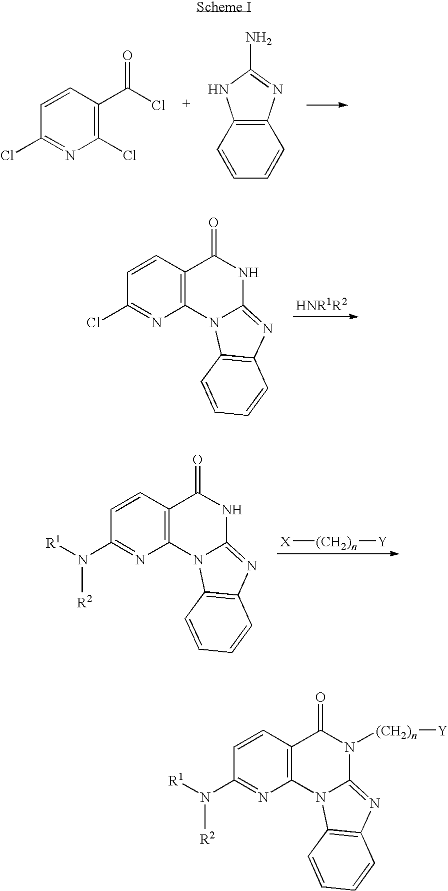

Scheme I is exemplified by Examples 1-4 herein, which provide a compound of formula (1) wherein Z¹ is N. As one of ordinary skill will recognize, other aromatic acyl chlorides such as suitably substituted benzoyl chlorides, isonicotinoyl chlorides, and picolinoyl chlorides can be substituted for the dichloronicotinoyl chloride shown in Scheme Ito provide compounds of formula (1) where Z¹-Z⁴ all represent carbon, or where Z² or Z³═N while Z¹ is C, or where Z¹ and Z³ are N, or where Z¹ and Z⁴ are N. Compounds of formula (1) having different substituents on that aromatic ring can also be prepared by starting with substituted aroyl chlorides. Similarly, other nucleophiles besides the amine HNR¹R² can be employed to introduce different substituents on that aromatic ring using a displacement such as that shown in the second step of the reaction in Scheme I, provided that a suitably positioned leaving group is present. As is known in the art, for example, a chloro or fluoro substituent on that aromatic ring can be displaced by a nucleophile such as an alkyl thiol; the alkylthio ether substituent of the product can then be oxidized to an alkylsulfonyl group, which can be displaced by other nucleophiles such as alkoxy groups, for example. In Examples 1-4, X—(CH₂)ₙ—Y represents 1-bromo-3-chloropropane; and after alkylation, the group Y must be replaced by an azacyclic group. However, other alkylating agents besides X—(CH₂)ₙ—Y can be employed to introduce groups having various linkers and azacyclic groups L-NR¹R² as shown in formula (1); Y in such alkylating agents may be the azacyclic group itself, or it may be a group that can be converted into an azacyclic group, such as a leaving group as illustrated in Examples 3-4.

Compounds of formula (2) wherein Z is N can be prepared from the first tetracyclic intermediate shown in Scheme I, as illustrated in Scheme II below.

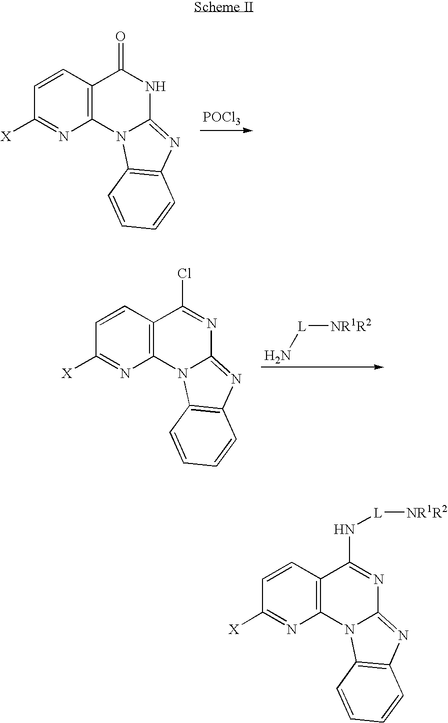

Compounds of formula (2) where Z is O or S can be similarly prepared from the corresponding oxygen or sulfur nucleophiles. And compounds having T=SO or SO₂ are readily prepared from the compound wherein T is S by oxidation methods known in the art.

As discussed above, the tetracyclic intermediate that is used as a starting material for this reaction can be made with other aroyl chlorides, to provide different ring systems; thus compounds of formula (2) having one or more of Z¹-Z⁴ representing N can be made in this fashion, as can compounds wherein each of Z¹-Z⁴ is C. In addition, where X represents a halogen, for example, X can be replaced using suitable nucleophiles, just as it was replaced in Scheme I with an amine.

Compounds of formula (3) wherein Z¹ is N can be prepared by the following synthetic scheme (Scheme III), which is exemplified in Examples 5-9 herein.

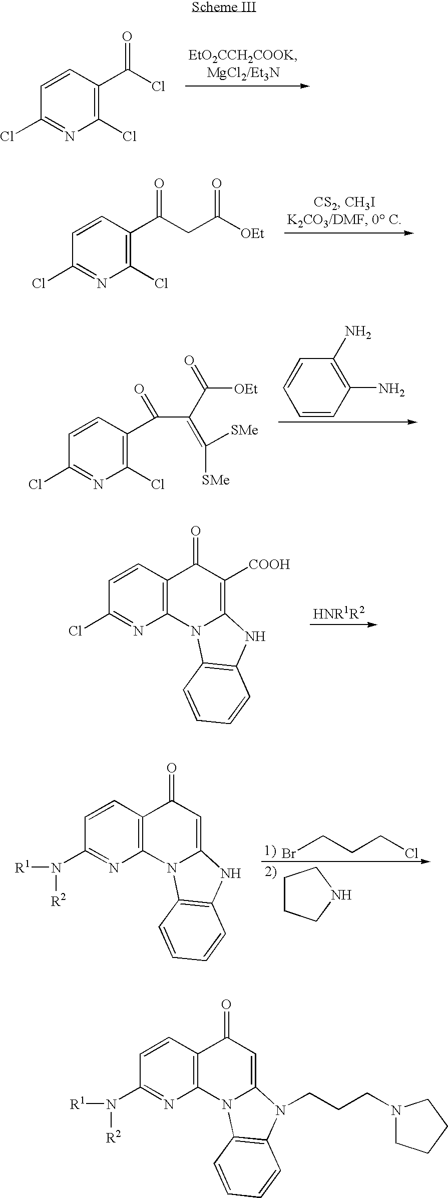

Again, other aroyl chlorides can be used as the starting materials for this synthesis scheme in order to provide other compounds of formula (3), and different nucleophiles such as other amines, aryl or alkyl thiols, and phenols can be introduced instead of the NR¹R² group depicted in Scheme III. Likewise, the alkylation step can be modified to introduce various other azacyclic groups as discussed above.

Scheme IV depicts a synthesis method to provide compounds of formula (2) wherein Z is CH. It utilizes an intermediate from Scheme III to introduce the amine substituent containing the L-NR¹R² group.

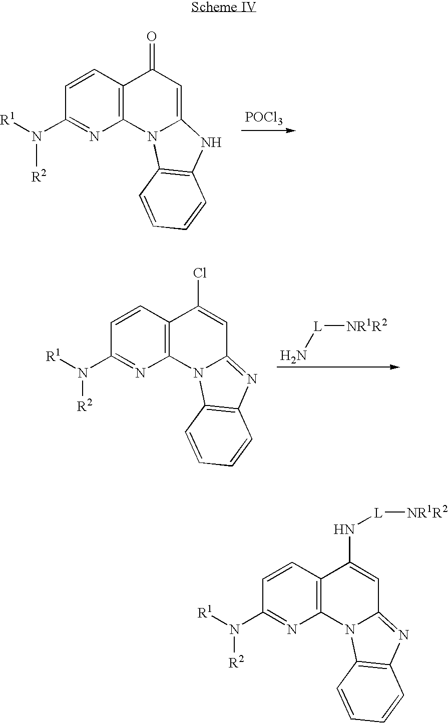

Again, other aroyl chlorides can be used as the starting materials for this synthesis scheme in order to provide other compounds of formula (2), and different nucleophiles such as other amines, aryl or alkyl thiols, and phenols can be introduced onto the pyridine ring instead of the NR¹R² group employed in Scheme III. Likewise, the amine introduction step that attaches the NH-L-NR¹R² group can be modified to introduce various other azacyclic groups as discussed above. Of course, the two separate NR¹R² groups are introduced independently and may be the same, but need not be the same and are often different.

Scheme V depicts a synthetic route to prepare compounds of formula (3) where Z is N, using a method similar to the one depicted above, in Scheme I. For this method, the azacyclic group is introduced into the starting benzimidazole. Introducing the substituent on the pyridinoyl ring can be accomplished directly as shown; but in some embodiments, it is preferable to first displace the chloro group with a thiol such as methanethiol, then oxidize the thioether to an alkysulfonyl group. That further activates the pyridine ring to facilitate its reaction with nucleophiles. Again, the NH-L-NR¹R² group can be varied and often comprises an azacyclic group as discussed above. Of course, the two separate NR¹R² groups are introduced independently and may be the same, but need not be the same and are often different; and a variety of other nucleophiles can be used instead of the amine on the pyridine ring.

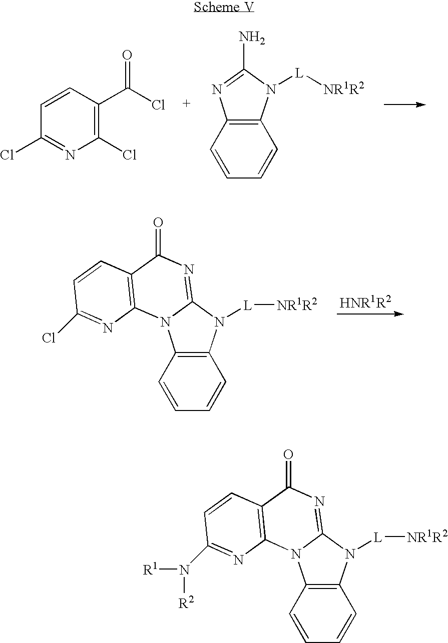

Variations and combinations of the foregoing reaction schemes can be used to prepare a wide variety of compounds of formula (1), (2), or (3), as well as compounds of formula (1a), (2a), or (3a), as will be appreciated by those of skill in the art.

The compounds of the present invention can be tested using screening assays such as those described herein. This enables one of ordinary skill to select a suitable compound for a particular application.

The compounds described herein may interact with regions of nucleic acids that can form quadruplexes. Because regions of DNA that can form quadruplexes are regulators of biological processes such as oncogene transcription, modulators of quadruplex biological activity can be utilized as cancer therapeutics. Molecules that interact with regions of DNA that can form quadruplexes can exert a therapeutic effect on certain cell proliferative disorders and related conditions. Particularly, abnormally increased oncogene expression can cause cell proliferative disorders, and quadruplex structures typically down-regulate oncogene expression. Examples of oncogenes include but are not limited to MYC, HIF, VEGF, ABL, TGF, PDGFA, MYB, SPARC, HUMTEL, HER, VAV, RET, H-RAS, EGF, SRC, BCL1, BCL2, DHFR, HMGA, and other oncogenes known to one of skill in the art. Furthermore, the compounds described herein may induce cell death (e.g., apoptosis) and not interact with regions of DNA that can form quadruplexes.

Molecules that bind to regions of DNA that can form quadruplexes can exert a biological effect according to different mechanisms, which include for example, stabilizing a native quadruplex structure, inhibiting conversion of a native quadruplex to duplex DNA by blocking strand cleavage, and stabilizing a native quadruplex structure having a quadruplex-destabilizing nucleotide substitution and other sequence specific interactions. Thus, compounds that bind to regions of DNA that can form quadruplexes described herein may be administered to cells, tissues, or organisms for the purpose of down-regulating oncogene transcription and thereby treating cell proliferative disorders.

Determining whether the biological activity of native DNA that can form quadruplexes is modulated in a cell, tissue, or organism can be accomplished by monitoring quadruplex biological activity. Quadruplex forming regions of DNA biological activity may be monitored in cells, tissues, or organisms, for example, by detecting a decrease or increase of gene transcription in response to contacting the quadruplex forming DNA with a molecule. Transcription can be detected by directly observing RNA transcripts or observing polypeptides translated by transcripts, which are methods well known in the art.

Molecules that interact with quadruplex forming DNA and quadruplex forming nucleic acids can be utilized to treat many cell proliferative disorders. Cell proliferative disorders include, for example, colorectal cancers and hematopoietic neoplastic disorders (i.e., diseases involving hyperplastic/neoplastic cells of hematopoietic origin such as those arising from myeloid, lymphoid or erythroid lineages, or precursor cells thereof). The diseases can arise from poorly differentiated acute leukemias, e.g., erythroblastic leukemia and acute megakaryoblastic leukemia. Additional myeloid disorders include, but are not limited to, acute promyeloid leukemia (APML), acute myelogenous leukemia (AML) and chronic myelogenous leukemia (CML) (Vaickus, Crit. Rev. in Oncol./Hemotol. 11:267-297 (1991)). Lymphoid malignancies include, but are not limited to acute lymphoblastic leukemia (ALL), which includes B-lineage ALL and T-lineage ALL, chronic lymphocytic leukemia (CLL), prolymphocytic leukemia (PLL), hairy cell leukemia (HLL) and Waldenstrom's macroglobulinemia (WM). Additional forms of malignant lymphomas include, but are not limited to non-Hodgkin lymphoma and variants thereof, peripheral T cell lymphomas, adult T cell leukemia/lymphoma (ATL), cutaneous T-cell lymphoma (CTCL), large granular lymphocytic leukemia (LGF), Hodgkin's disease and Reed-Sternberg disease. Cell proliferative disorders also include cancers of the colorectum, breast, lung, liver, pancreas, lymph node, colon, prostate, brain, head and neck, skin, liver, kidney, and heart. Compounds that interact with regions of DNA that may form quadruplexes also can be utilized to target cancer related processes and conditions, such as increased angiogenesis, by inhibiting angiogenesis in a subject.

The present invention provides a method for reducing cell proliferation or for treating or alleviating cell proliferative disorders, comprising contacting a system having a native DNA capable of forming a quadruplex region with a compound having any one of the above formula. The system may be a group of cells or one or more tissues. In one embodiment, the system is a subject in need of a treatment of a cell proliferative disorder (e.g., a mammal such as a mouse, rat, monkey, or human). The present invention also provides a method for treating colorectal cancer by administering a compound that interacts with a c-MYC quadruplex forming region to a subject in need thereof, thereby reducing the colorectal cancer cell proliferation. Furthermore, the present invention provides a method for inhibiting angiogenesis and optionally treating a cancer associated with angiogenesis, comprising administering a compound that interacts with a vascular endothelial growth factor (VEGF) quadruplex forming region to a subject in need thereof, thereby reducing angiogenesis and optionally treating a cancer associated with angiogenesis.

Compounds that interact with quadruplex forming regions of DNA can also be used to reduce a microbial infection, such as a viral infection. Retroviruses offer a wealth of potential targets for G-quadruplex targeted therapeutics. G-quadruplex structures have been implicated as functional elements in at least two secondary structures formed by either viral RNA or DNA in HIV, the dimer linker structure (DLS) and the central DNA flap (CDF). Additionally, DNA aptamers which are able to adopt either inter- or intramolecular quadruplex structures are able to inhibit viral replication. In one example, DNA aptamers are able to inhibit viral replication by targeting the envelope glycoprotein (putatively). In another example, DNA aptamers inhibit viral replication by targeting the HIV-integrase respectively, suggesting the involvement of native quadruplex structures in interaction with the integrase enzyme.

Dimer linker structures, which are common to all retroviruses, serve to bind two copies of the viral genome together by a non-covalent interaction between the two 5′ ends of the two viral RNA sequences. The genomic dimer is stably associated with the gag protein in the mature virus particle. In the case of HIV, the origin of this non-covalent binding may be traced to a 98 base-pair sequence containing several runs of at least two consecutive guanines (e.g., the 3′ for the formation of RNA dimers in vitro). An observed cation (potassium) dependence for the formation and stability of the dimer in vitro, in addition to the failure of an antisense sequence to effectively dimerize, has revealed the most likely binding structure to be an intermolecular G-quadruplex.

Prior to integration into the host genome, reverse transcribed viral DNA forms a pre-integration complex (PIC) with at least two major viral proteins, integrase and reverse transcriptase, which is subsequently transported into the nucleus. The Central DNA Flap (CDF) refers to 99-base length single-stranded tail of the + strand, occurring near the center of the viral duplex DNA, which is known to a play a role in the nuclear import of the PIC. Oligonucleotide mimics of the CDF have been shown to form intermolecular G-quadruplex structures in cell-free systems.

Thus, compounds that recognize quadruplex forming regions can be used to stabilize the dimer linker structure and thus prevent de-coupling of the two RNA strands. Also, by binding to the quadruplex structure formed by the CDF, protein recognition and/or binding events for nuclear transport of the PIC may be disrupted. In either case, a substantial advantage can exist over other anti-viral therapeutics. Current Highly Active Anti-Retroviral Therapeutic (HAART) regimes rely on the use of combinations of drugs targeted towards the HIV protease and HIV integrase. The requirement for multi-drug regimes is to minimize the emergence of resistance, which will usually develop rapidly when agents are used in isolation. The source of such rapid resistance is the infidelity of the reverse transcriptase enzyme which makes a mutation approximately once in every 10,000 base pairs. An advantage of targeting viral quadruplex structures over protein targets, is that the development of resistance is slow or is impossible. A point mutation of the target quadruplex can compromise the integrity of the quadruplex structure and lead to a non-functional copy of the virus. A single therapeutic agent based on this concept may replace the multiple drug regimes currently employed, with the concomitant benefits of reduced costs and the elimination of harmful drug/drug interactions.

The present invention provides a method for reducing a microbial titer in a system, comprising contacting a system having a native DNA quadruplex forming region with a compound having any one of the above formula. The system may be one or more cells or tissues. Examples of microbial titers include but are not limited to viral, bacterial or fungal titers. In a particular embodiment, the system is a subject in need of a treatment for a viral infection (e.g., a mammal such as a mouse, rat, monkey, or human). Examples of viral infections include infections by a hepatitis virus (e.g., hepatitis B or C), human immunodeficiency virus (HIV), rhinovirus, herpes-zoster virus (VZV), herpes simplex virus (e.g., HSV-1 or HSV-2), cytomegalovirus (CMV), vaccinia virus, influenza virus, encephalitis virus, hantavirus, arbovirus, West Nile virus, human papilloma virus (HPV), Epstein-Barr virus, and respiratory syncytial virus. The present invention also provides a method for treating HIV infection by administering a compound having any one of the above formula to a subject in need thereof, thereby reducing the HIV infection.

Identifying Compounds that can Bind to Quadruplex Forming Regions of DNA

Compounds described herein may bind to quadruplex forming regions of DNA where a biological activity of this region, often expressed as a “signal,” produced in a system containing the compound is different than the signal produced in a system not containing the compound. While background signals may be assessed each time a new molecule is probed by the assay, detecting the background signal is not required each time a new molecule is assayed.

Examples of quadruplex forming nucleotide sequences are set forth in the following Table 2:

| | SEQ | |
|---|---|---|
| | ID | |
| SEQUENCE | NO | ORIGIN |
| TG₄AG₃TG₄AG₃TG₄AAGG | 1 | CMYC |
| GGGGGGGGGGGGGCGGGGGCGGGGGCGGGGGAGGGGC | 2 | PDGFA |
| G₈ACGCG₃AGCTG₅AG₃CTTG₄CCAG₃CG₄CGCTTAG₅ | 3 | PDGFB/ |
| | | c-sis |
| AGGAAGGGGAGGGCCGGGGGGAGGTGGC | 4 | CABL |
| AGGGGCGGGGCGGGGCGGGGGC | 5 | RET |
| AG₄CG₃CGCGGGAGGAAGGGGGCGGGAGCGGGGCTG | 6 | BCL-2 |
| GGGGGGCGGGGGCGGGCGCAGGGGGAGGGGGC | 7 | Cyclin |
| | | D1/BCL-1 |
| CGGGGCGGGGCGGGGGCGGGGGC | 8 | H-RAS |
| AGAGGAGGAGGAGGTCACGGAGGAGGAGGAGAAGG | 9 | CMYB |
| AGGAGGAGGAA or | | |
| AGAGGAGGAGGAGGACACGGAGGAGGAGGAGAAG | | |
| GAGGAGGAGGAA | | |
| (GGA)₄ | 10 | VAV |
| AGAGAAGAGGGGAGGAGGAGGAGGAGAGGAGGAGGCGC | 11 | HMGA2 |
| GGAGGGGGAGGGG | 12 | CPIM |
| AGGAGAAGGAGGAGGTGGAGGAGGAGG | 13 | HER2/ |
| | | neu |
| AGGAGGAGGAGAATGCGAGGAGGAGGGAGGAGA | 14 | EGFR |
| GGGGCGGGCCGGGGGCGGGGTCCCGGCGGGGCGGAG | 15 | VEGF |
| CGGGAGGAGGAGGAAGGAGGAAGCGCG | 16 | CSRC |

In addition to determining whether a test molecule or test nucleic acid gives rise to a different signal, the affinity of the interaction between the nucleic acid and the compound may be quantified. IC₅₀, K_{d}, or Kᵢ threshold values may be compared to the measured IC₅₀ or K_{d} values for each interaction, and thereby identify a test molecule as a quadruplex interacting molecule or a test nucleic acid as a quadruplex forming nucleic acid. For example, IC₅₀ or K_{d} threshold values of 10 μM or less, 1 μM or less, and 100 nM or less are often utilized. In another example, threshold values of 10 nM or less, 1 nM or less, 100 μM or less, and 10 μM or less may be utilized to identify quadruplex interacting molecules and quadruplex forming nucleic acids.

Many assays are available for identifying compounds that have affinity for quadruplex forming regions of DNA. In some of these assays, the biological activity is the quadruplex nucleic acid binding to a compound and binding is measured as a signal. In other assays, the biological activity is a polymerase arresting function of a quadruplex and the degree of arrest is measured as a decrease in a signal. In certain assays, the biological activity is transcription and transcription levels can be quantified as a signal. In another assay, the biological activity is cell death and the number of cells undergoing cell death is quantified. Another assay monitors proliferation rates of cancer cells. Examples of assays are fluorescence binding assays, gel mobility shift assays (see, e.g., Jin & Pike, Mol. Endocrinol. (1996) 10:196-205), polymerase arrest assays, transcription reporter assays, cancer cell proliferation assays, and apoptosis assays (see, e.g., Amersham Biosciences (Piscataway, N.J.)), and embodiments of such assays are described hereafter. Also, topoisomerase assays can be utilized to determine whether the quadruplex interacting molecules have a topoisomerase pathway activity (see, e.g., TopoGEN, Inc. (Columbus, Ohio)).

### Gel Electrophoretic Mobility Shift Assay (EMSA)

An EMSA is useful for determining whether a nucleic acid forms a quadruplex and whether a nucleotide sequence is quadruplex-destabilizing. EMSA is conducted as described previously (Jin & Pike, Mol. Endocrinol. 10: 196-205 (1996)) with minor modifications. Generally, synthetic single-stranded oligonucleotides are labeled in the 5′ terminus with T4-kinase in the presence of [γ-³²P] ATP (1,000 mCi/mmol, Amersham Life Science) and purified through a sephadex column. ³²P-labeled oligonucleotides (30,000 cpm) are then incubated with or without various concentrations of a testing compound in 20 μl of a buffer containing 10 mM Tris pH 7.5, 100 mM KCl, 5 mM dithiothreitol, 0.1 mM EDTA, 5 mM MgCl₂, 10% glycerol, 0.05% Nonedit P-40, and 0.1 mg/ml of poly(dI-dC) (Pharmacia). After incubation for 20 minutes at room temperature, binding reactions are loaded on a 5% polyacrylamide gel in 0.25×Tris borate-EDTA buffer (0.25×TBE, 1×TBE is 89 mM Tris-borate, pH 8.0, 1 mM EDTA). The gel is dried and each band is quantified using a phosphoimager.

### DMS Methylation Protection Assay

Chemical footprinting assays are useful for assessing quadruplex structure. Quadruplex structure is assessed by determining which nucleotides in a nucleic acid are protected or unprotected from chemical modification as a result of being inaccessible or accessible, respectively, to the modifying reagent. A DMS methylation assay is an example of a chemical footprinting assay. In such an assay, bands from EMSA are isolated and subjected to DMS-induced strand cleavage. Each band of interest is excised from an electrophoretic mobility shift gel and soaked in 100 mM KCl solution (300 μl) for 6 hours at 4° C. The solutions are filtered (microcentrifuge) and 30,000 cpm (per reaction) of DNA solution is diluted further with 100 mM KCl in 0.1×TE to a total volume of 70 μl (per reaction). Following the addition of 1 μl salmon sperm DNA (0.1 μg/μl), the reaction mixture is incubated with 1 μl DMS solution (DMS:ethanol; 4:1; v:v) for a period of time. Each reaction is quenched with 18 μl of stop buffer (b-mercaptoethanol:water:NaOAc (3 M); 1:6:7; v:v:v). Following ethanol precipitation (twice) and piperidine cleavage, the reactions are separated on a preparative gel (16%) and visualized on a phosphoimager.

### Polymerase Arrest Assay

An arrest assay includes a template nucleic acid, which may comprise a quadruplex forming sequence, and a primer nucleic acid which hybridizes to the template nucleic acid 5′ of the quadruplex-forming sequence. The primer is extended by a polymerase (e.g., Taq polymerase), which advances from the primer along the template nucleic acid. In this assay, a quadruplex structure can block or arrest the advance of the enzyme, leading to shorter transcription fragments. Also, the arrest assay may be conducted at a variety of temperatures, including 45° C. and 60° C., and at a variety of ion concentrations.

An example of the Taq polymerase stop assay is described in Han, et al., Nucl. Acids Res. (1999) 27:537-542, which is a modification of that used by Weitzmann, et al., J. Biol. Chem. (1996) 271:20958-20964. Briefly, a reaction mixture of template DNA (50 nM), Tris.HCl (50 mM), MgCl₂ (10 mM), DTT (0.5 mM), EDTA (0.1 mM), BSA (60 ng), and 5′-end-labeled quadruplex nucleic acid (−18 nM) is heated to 90° C. for 5 minutes and allowed to cool to ambient temperature over 30 minutes. Taq Polymerase (1 μl) is added to the reaction mixture, and the reaction is maintained at a constant temperature for 30 minutes. Following the addition of 10 μl stop buffer (formamide (20 ml), 1 M NaOH (200 μl), 0.5 M EDTA (400 μl), and 10 mg bromophenol blue), the reactions are separated on a preparative gel (12%) and visualized on a phosphoimager. Adenine sequencing (indicated by “A” at the top of the gel) is performed using double-stranded DNA Cycle Sequencing System from Life Technologies. The general sequence for the template strands is TCCAACTATGTATAC-INSERT-TTAGCGACACGCAATTGCTATAGTGAGTCGTATTA, where “INSERT” refers to a nucleic acid sequence comprising a quadruplex forming sequence (See e.g., Table 2). Bands on the gel that exhibit slower mobility are indicative of quadruplex formation.

### High Throughput Polymerase Arrest Assay

A high throughput polymerase arrest assay has been developed. The assay comprises contacting a template nucleic acid, often DNA, with a primer, which also is often DNA; contacting the primer/template complex with a compound described herein (also referred to as a “test compound”); contacting the primer/template complex with a polymerase; and separating reaction products. The assay often includes the step of denaturing the primer/template complex mixture and then renaturing the complex, which often is carried out before a test molecule is added to the system. Multiple assays often are carried out using varying concentrations of a test compound, such that an IC₅₀ value can be obtained, for example. The reaction products often include extended primers of different lengths. Where a test compound does not significantly interact with a quadruplex structure in the template, the primer often is extended to the end of the template.

Where a test compound significantly interacts with a quadruplex structure in the template, the primer often is extended only to the quadruplex structure in the template and no further. Thus, the reaction mixture often includes at least two reaction products when a test compound interacts with a quadruplex structure in the template, one having a completely extended primer and one having an incompletely extended primer, and these two reaction products are separated. The products may be separated using any convenient separation method, such as mass spectrometry and in one embodiment, capillary electrophoresis.

The reaction products often are identified by detecting a detectable label linked to the primer. The detectable label may be non-covalently linked to the 5′ end of the primer (e.g., a biotin molecule covalently linked to the 5′ end of the primer which is non-covalently linked to an avidin molecule joined to a detectable label). The detectable label may be joined to the primer at any stage of the assay, sometimes before the primer is added to the system, after the primer is extended, or after the products are separated. The detectable label often is covalently linked to the primer using a procedure selected based upon the nature of the chemical groups in the detectable label.

Many methods for covalently linking detectable labels to nucleic acids are available, such as chemically coupling an allylamine-derivatized nucleotide to a succinimidyl-ester derivative of a detectable label, and then generating a primer using the labeled nucleotide. (See, e.g., Nature Biotech (2000) 18:345-348 and http address info.med.yale.edu/genetics/ward/tavi/n_coupling.html). A spacer (often between 5-16 carbon atoms long) sometimes is incorporated between the detectable label and the nucleotide. Any convenient detectable label may be utilized, including but not limited to a radioactive isotope (e.g., ¹²⁵I, ¹³¹I, ³⁵S, ³²P, ¹⁴C or ³H) a light scattering label (e.g., a spherical gold or silver label; Genicon Sciences Corporation, San Diego, Calif. and U.S. Pat. No. 6,214,560); an enzymic or protein label (e.g., GFP or peroxidase); or another chromogenic label or dye sometimes is utilized. Often, a fluorescent label is utilized (e.g., amino-methyl coumarin (AMCA); diethyl aminomethyl coumarin (DEAC); cascade blue (CB); fluorescein isothiocyanate (FITC); Oregon green (OG); Alexa 488 (A488); rhodamine green (RGr); lanthanide chelate (e.g., europium), carboxy-rhodamine 6G (R6G); tetramethyl rhodamine (TAMRA); Texas Red (TxR); Cy3; Cy3.5; Cy5, Cy5.5 and carboxynaphtofluorescein (CNF), digoxigenin (DIG); and 2,4-dinitrophenyl (DNP)). Other fluorophores and attendant excitation and emission wavelengths are described in Anantha, et al., Biochemistry (1998) 37:2709-2714 and Qu & Chaires, Methods Enzymol (2000) 321:353-369).

In an embodiment, a primer oligonucleotide covalently linked to a fluorescent label is contacted with template DNA. The resulting complex is contacted with a test molecule and then contacted with a polymerase capable of extending the primer. The reaction products then are separated and detected by capillary electrophoresis. A longer primer sequence was used for practicing this embodiment as compared to embodiments where the primer includes no covalently-linked fluorophore or where capillary electrophoresis is not utilized for separation. Deoxynucleotides are added at any stage of the assay before the separation, often when the primer is contacted with the template DNA. The template DNA/primer complex often is denatured (e.g., by increasing the temperature of the system) and then renatured (e.g., by cooling the system) before a test compound is added).

### Quadruplex Binding Assay

Generally, a 5′-fluorescent-labeled (FAM) primer (P45, 15 nM) was mixed with template DNA (15 nM) in a Tris-HCL buffer (15 mM Tris, pH 7.5) containing 10 mM MgCl₂, 0.1 mM EDTA and 0.1 mM mixed deoxynucleotide triphosphates (dNTP's). In one example, the FAM-P45 primer (5′-6FAM-AGTCTGACTGACTGTACGTAGCTAATACGACTCACTATAG CAATT-3′) (SEQ ID NO. 17) and the c-Myc template DNA (5′-TCCAACTATGTATACTGGGG AGGGTGGGGAGGGTGGGGAAGGTTAGCGACACGCAATTGCTATAGTGAGTCGTAT TAGCTACGTACAGTCAGTCAGACT-3′) (SEQ ID NO. 18) were synthesized and HPLC purified by Applied Biosystems. The mixture was denatured at 95° C. for 5 minutes and, after cooling down to room temperature, was incubated at 37° C. for 15 minutes.

After cooling down to room temperature, 1 mM KCl and the test compound (various concentrations) were added and the mixture incubated for 15 minutes at room temperature. The primer extension was performed by adding 10 mM KCl and Taq DNA Polymerase (2.5 U/reaction, Promega) and incubating at 70° C. for 30 minutes. The reaction was stopped by adding 1 μl of the reaction mixture to 10 μl Hi-Di Formamide mixed and 0.25 μl LIZ120 size standard. Hi-Di Formamide and LIZ120 size standard were purchased from Applied Biosystems. The partially extended quadruplex arrest product was between 61 or 62 bases long and the full-length extended product was 99 bases long. The products were separated and analyzed using capillary electrophoresis. Capillary electrophoresis was performed using an ABI PRISM 3100-Avant Genetic Analyzer. The assay was performed using compounds described above and results are shown in Table 1. μM concentrations reported in Table 1 are concentrations at which 50% of the DNA was arrested in the assay (i.e., the ratio of shorter partially extended DNA (arrested DNA) to full-length extended DNA is 1:1).

### Transcription Reporter Assay

In a transcription reporter assay, test quadruplex DNA is coupled to a reporter system, such that a formation or stabilization of a quadruplex structure can modulate a reporter signal. An example of such a system is a reporter expression system in which a polypeptide, such as luciferase or green fluorescent protein (GFP), is expressed by a gene operably linked to the potential quadruplex forming nucleic acid and expression of the polypeptide can be detected. As used herein, the term “operably linked” refers to a nucleotide sequence which is regulated by a sequence comprising the potential quadruplex forming nucleic acid. A sequence may be operably linked when it is on the same nucleic acid as the quadruplex DNA, or on a different nucleic acid. An exemplary luciferase reporter system is described herein.

A luciferase promoter assay described in He, et al., Science (1998) 281:1509-1512 often is utilized for the study of quadruplex formation. Specifically, a vector utilized for the assay is set forth in reference 11 of the He, et al., document. In this assay, HeLa cells are transfected using the lipofectamin 2000-based system (Invitrogen) according to the manufacturer's protocol, using 0.1 μg of pRL-TK (Renilla luciferase reporter plasmid) and 0.9 μg of the quadruplex-forming plasmid. Firefly and Renilla luciferase activities are assayed using the Dual Luciferase Reporter Assay System (Promega) in a 96-well plate format according to the manufacturer's protocol.

### Circular Dichroism Assay

Circular dichroism (CD) is utilized to determine whether another molecule interacts with a quadruplex nucleic acid. CD is particularly useful for determining whether a PNA or PNA-peptide conjugate hybridizes with a quadruplex nucleic acid in vitro. PNA probes are added to quadruplex DNA (5 μM each) in a buffer containing 10 mM potassium phosphate (pH 7.2) and 10 or 250 mM KCl at 37° C. and then allowed to stand for 5 minutes at the same temperature before recording spectra. CD spectra are recorded on a Jasco J-715 spectropolarimeter equipped with a thermoelectrically controlled single cell holder. CD intensity normally is detected between 220 nm and 320 nm and comparative spectra for quadruplex DNA alone, PNA alone, and quadruplex DNA with PNA are generated to determine the presence or absence of an interaction (see, e.g., Datta, et al., JACS (2001) 123:9612-9619). Spectra are arranged to represent the average of eight scans recorded at 100 nm/min.

### Fluorescence Binding Assay

An example of a fluorescence binding assay is a system that includes a quadruplex nucleic acid, a signal molecule, and a test molecule. The signal molecule generates a fluorescent signal when bound to the quadruplex nucleic acid (e.g., N-methylmesoporphyrin IX (NMM)), and the signal is altered when a test compound competes with the signal molecule for binding to the quadruplex nucleic acid. An alteration in the signal when test molecule is present as compared to when test compound is not present identifies the test compound as a quadruplex interacting compound.

50 μl of quadruplex nucleic acid or a nucleic acid not capable of forming a quadruplex is added in 96-well plate. A test compound also is added in varying concentrations. A typical assay is carried out in 100 μl of 20 mM HEPES buffer, pH 7.0, 140 mM NaCl, and 100 mM KCl. 50 μl of the signal molecule NMM then is added for a final concentration of 3 μM. NMM is obtained from Frontier Scientific Inc, Logan, Utah. Fluorescence is measured at an excitation wavelength of 420 nm and an emission wavelength of 660 nm using a FluoroStar 2000 fluorometer (BMG Labtechnologies, Durham, N.C.). Fluorescence often is plotted as a function of concentration of the test compound or quadruplex-targeted nucleic acid and maximum fluorescent signals for NMM are assessed in the absence of these molecules.

### Cell Proliferation Assay

In a cancer cell proliferation assay, cell proliferation rates are assessed as a function of different concentrations of test compounds added to the cell culture medium. Any cancer cell type can be utilized in the assay. In one embodiment, colon cancer cells are cultured in vitro and test compounds are added to the culture medium at varying concentrations. A useful colon cancer cell line is colo320, which is a colon adenocarcinoma cell line deposited with the National Institutes of Health as accession number JCRB0225. Parameters for using such cells are available at the http address cellbank.nihs.go.jp/cell/data/jcrb0225.htm.

Formulation of Compounds

As used herein, the term “pharmaceutically acceptable salts, esters and amides” includes but are not limited to carboxylate salts, amino acid addition salts, esters and amides of the compounds, as well as the zwitterionic forms thereof, which are known to those skilled in the art as suitable for use with humans and animals. (See, e.g., Gerge, S. M., et al., “Pharmaceutical Salts,” J. Pharm. Sci. (1977) 66:1-19, which is incorporated herein by reference.)

Any suitable formulation of the compounds described herein can be prepared using carriers and excipients that are well known in the art for use in a particular application. For example, compounds may be admixed with a carrier for use in in vitro or in vivo applications. Suitable carriers include partially purified water, such as deionized water or an isotonic solution; buffer systems such as bicarbonate, phosphate, and similar buffers; and mixtures of aqueous solutions with water-miscible organic cosolvents such as acetone or DMSO. Phosphate-buffered saline (PBS), which may be buffered to provide a neutral pH, or in certain embodiments an acidic pH, is sometimes preferred. Stabilizing agents may also be included.

In cases where compounds are sufficiently basic or acidic to form stable nontoxic acid or base salts, administration of the compounds as salts may be appropriate. Examples of pharmaceutically acceptable salts are organic acid addition salts formed with acids that form a physiological acceptable anion, for example, tosylate, methanesulfonate, acetate, citrate, malonate, tartarate, succinate, benzoate, ascorbate, α-ketoglutarate, and α-glycerophosphate. Suitable inorganic salts may also be formed, including hydrochloride, sulfate, nitrate, bicarbonate, and carbonate salts. Pharmaceutically acceptable salts are obtained using standard procedures well known in the art. For example, pharmaceutically acceptable salts may be obtained by reacting a sufficiently basic compound such as an amine with a suitable acid affording a physiologically acceptable anion. Alkali metal (e.g., sodium, potassium or lithium) or alkaline earth metal (e.g., calcium, magnesium) salts of carboxylic acids and other anionic groups in molecules within the invention also are contemplated.

A compound may be formulated as a pharmaceutical composition and administered to a mammalian host in need of such treatment. For pharmaceutical applications, a compound is typically combined with a pharmaceutically acceptable carrier such as water, an isotonic solution, or PBS. Other pharmaceutically acceptable excipients may also be included. In one embodiment, the mammalian host is human. Any suitable route of administration may be used, including but not limited to oral, parenteral, intravenous, intramuscular, topical and subcutaneous routes.

In one embodiment, a compound is administered systemically (e.g., orally) in combination with a pharmaceutically acceptable vehicle such as an inert diluent or an assimilable edible carrier. They may be enclosed in hard or soft shell gelatin capsules, compressed into tablets, or incorporated directly with the food of the patient's diet. For oral therapeutic administration, the active compound may be combined with one or more excipients and used in the form of ingestible tablets, buccal tablets, troches, capsules, elixirs, suspensions, syrups, wafers, and the like. Such compositions and preparations should contain at least 0.1% of active compound. The percentage of the compositions and preparations may be varied and may conveniently be between about 2 to about 60% of the weight of a given unit dosage form. The amount of active compound in such therapeutically useful compositions is such that an effective dosage level will be obtained.

Tablets, troches, pills, capsules, and the like also may contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Any material used in preparing any unit dosage form is pharmaceutically acceptable and substantially non-toxic in the amounts employed. In addition, the active compound may be incorporated into sustained-release preparations and devices.

The active compound also may be administered intravenously or intraperitoneally by infusion or injection. Solutions of the active compound or its salts may be prepared in a buffered solution, often phosphate buffered saline, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms. The compound is sometimes prepared as a polymatrix-containing formulation for such administration (e.g., a liposome or microsome). Liposomes are described for example in U.S. Pat. No. 5,703,055 (Felgner, et al.) and Gregoriadis, Liposome Technology vols. I to III (2nd ed. 1993).

The pharmaceutical dosage forms suitable for injection or infusion can include sterile aqueous solutions or dispersions or sterile powders comprising the active ingredient that are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating the active compound in the required amount in the appropriate solvent with various of the other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

For topical administration, the present compounds may be applied in liquid form. Compounds often are administered as compositions or formulations, in combination with a dermatologically acceptable carrier, which may be a solid or a liquid. Examples of useful dermatological compositions used to deliver compounds to the skin are known (see, e.g., Jacquet, et al. (U.S. Pat. No. 4,608,392), Geria (U.S. Pat. No. 4,992,478), Smith, et al. (U.S. Pat. No. 4,559,157) and Wortzman (U.S. Pat. No. 4,820,508).

Compounds may be formulated with a solid carrier, which include finely divided solids such as talc, clay, microcrystalline cellulose, silica, alumina and the like. Useful liquid carriers include water, alcohols or glycols or water-alcohol/glycol blends, in which the present compounds can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers. Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Generally, the concentration of the compound in a liquid composition often is from about 0.1 wt % to about 25 wt %, sometimes from about 0.5 wt % to about 10 wt %. The concentration in a semi-solid or solid composition such as a gel or a powder often is about 0.1 wt % to about 5 wt %, sometimes about 0.5 wt % to about 2.5 wt %. Higher concentrations are also appropriate for some solid or semi-solid compositions, and may include amounts up to about 25 wt % or up to about 50 wt % or more. A compound composition may be prepared as a unit dosage form, which is prepared according to conventional techniques known in the pharmaceutical industry. In general terms, such techniques include bringing a compound into association with pharmaceutical carrier(s) and/or excipient(s) in liquid form or finely divided solid form, or both, and then shaping the product if required.

Table 3 shows examples of formulations for use with compounds described herein. For example, a compound may be formulated having dosages from 10 mg/mL to 20 mg/mL solution, using the formulations herein. In Table 3, the designation “D5W” refers to deionized water with 5% dextrose. Each component in each formulation may be varied without affecting the activity of the compound. In one example, the compound is formulated in a solution comprising polyethylene glycol and propylene glycol in a buffer solution such as a phosphate buffer.

TABLE 3

| | | | Compound | pH of | pH of the |
|---|---|---|---|---|---|
| | | | (mL) + | the | formulated |
| | | % | Placebo | Placebo | solution |
| | Formulations | (w/w) | solution (mL) | solution | (10 mg/mL) |
| 1. | Mannitol | 4 | 35 ml + 35 mL | 6.1 | 6.1 |
| | Sucrose | 0.5 | | | |
| | 5% D5W solution | 95.5 | | | |
| 2. | Mannitol | 4 | 35 ml + 35 mL | 6 | 5.8 |
| | 50 mM PO₄ buffer, | 96 | | | |
| | pH = 6.0 | | | | |
| 3. | Mannitol | 4 | 35 ml + 35 mL | 5 | 5 |
| | 50 mM Citrate | 96 | | | |
| | buffer, pH = 5.0 | | | | |
| 4. | Mannitol | 4 | 35 ml + 35 mL | 6 | 6 |
| | 5% D5W | 96 | | | |
| 5. | Test compound | 1 | 35 ml + 35 mL | 6.4 | 6.1 |
| | (20 mg/mL) | | | | |
| | 5% D5W | 99 | | | |
| 6. | PEG 300 | 7 | 5 ml + 5 mL | N/A | 5.80 |
| | Propylene glycol | 9 | | | |
| | 5% D5W | 84 | | | |
| 7. | PEG 300 | 7 | 5 ml + 5 mL | N/A | 5.8 |
| | Propylene glycol | 9 | | | |
| | 50 mM PO₄ buffer, | 84 | | | |
| | pH = 6.0 | | | | |
| 8. | Mannitol | 4 | 5 ml + 5 mL | N/A | 5.7 |
| | PEG 300 | 20 | | | |
| | 50 mM PO₄ buffer, | 76 | | | |
| | pH = 6.0 | | | | |
| 9. | Mannitol | 4 | 5 ml + 5 mL | N/A | 5.8 |
| | Propylene glycol | 10 | | | |
| | 50 mM PO₄ buffer, | 86 | | | |
| | pH = 6.0 | | | | |

The compound composition may be formulated into any dosage form, such as tablets, capsules, gel capsules, liquid syrups, soft gels, suppositories, and enemas. The compositions also may be formulated as suspensions in aqueous, non-aqueous, or mixed media. Aqueous suspensions may further contain substances which increase viscosity, including for example, sodium carboxymethylcellulose, sorbitol, and/or dextran. The suspension may also contain one or more stabilizers. The amount of the compound, or an active salt or derivative thereof, required for use in treatment will vary not only with the particular salt selected but also with the route of administration, the nature of the condition being treated and the age and condition of the patient and will be ultimately at the discretion of the attendant physician or clinician.

Dosages

A useful compound dosage often is determined by assessing its in vitro activity in a cell or tissue system and/or in vivo activity in an animal system. For example, methods for extrapolating an effective dosage in mice and other animals to humans are known to the art (see, e.g., U.S. Pat. No. 4,938,949). Such systems can be used for determining the LD₅₀ (the dose lethal to 50% of the population) and the ED₅₀ (the dose therapeutically effective in 50% of the population) of a compound. The dose ratio between a toxic and therapeutic effect is the therapeutic index and it can be expressed as the ratio ED₅₀/LD₅₀. The compound dosage often lies within a range of circulating concentrations for which the ED₅₀ is associated with little or no toxicity. The dosage may vary within this range depending upon the dosage form employed and the route of administration utilized. For any compounds used in the methods described herein, the therapeutically effective dose can be estimated initially from cell culture assays. A dose sometimes is formulated to achieve a circulating plasma concentration range covering the IC₅₀ (i.e., the concentration of the test compound which achieves a half-maximal inhibition of symptoms) as determined in in vitro assays, as such information often is used to more accurately determine useful doses in humans. Levels in plasma may be measured, for example, by high performance liquid chromatography.

Another example of effective dose determination for a subject is the ability to directly assay levels of “free” and “bound” compound in the serum of the test subject. Such assays may utilize antibody mimics and/or “biosensors” generated by molecular imprinting techniques. The compound is used as a template, or “imprinting molecule”, to spatially organize polymerizable monomers prior to their polymerization with catalytic reagents. Subsequent removal of the imprinted molecule leaves a polymer matrix which contains a repeated “negative image” of the compound and is able to selectively rebind the molecule under biological assay conditions (see, e.g., Ansell, et al., Current Opinion in Biotechnology (1996) 7:89-94 and in Shea, Trends in Polymer Science (1994) 2:166-173).

Such “imprinted” affinity matrixes are amenable to ligand-binding assays, whereby the immobilized monoclonal antibody component is replaced by an appropriately imprinted matrix (see, e.g., Vlatakis, et al., Nature (1993) 361:645-647). Through the use of isotope-labeling, “free” concentration of compound can be readily monitored and used in calculations of IC₅₀. Such “imprinted” affinity matrixes can also be designed to include fluorescent groups whose photon-emitting properties measurably change upon local and selective binding of compound. These changes can be readily assayed in real time using appropriate fiberoptic devices, in turn allowing the dose in a test subject to be quickly optimized based on its individual IC₅₀. An example of such a “biosensor” is discussed in Kriz, et al., Analytical Chemistry (1995) 67:2142-2144.

Exemplary doses include milligram or microgram amounts of the compound per kilogram of subject or sample weight, for example, about 1 microgram per kilogram to about 500 milligrams per kilogram, about 100 micrograms per kilogram to about 5 milligrams per kilogram, or about 1 microgram per kilogram to about 50 micrograms per kilogram. It is understood that appropriate doses of a small molecule depend upon the potency of the small molecule with respect to the expression or activity to be modulated. When one or more of these small molecules is to be administered to an animal (e.g., a human) in order to modulate expression or activity of a polypeptide or nucleic acid described herein, a physician, veterinarian, or researcher may, for example, prescribe a relatively low dose at first, subsequently increasing the dose until an appropriate response is obtained. In addition, it is understood that the specific dose level for any particular animal subject will depend upon a variety of factors including the activity of the specific compound employed, the age, body weight, general health, gender, and diet of the subject, the time of administration, the route of administration, the rate of excretion, any drug combination, and the degree of expression or activity to be modulated.

Many compounds of the invention or precursors thereof may be prepared by combinations and/or modifications of synthetic transformations that are well known in the art. The following examples are offered to illustrate but not to limit the invention.

### Example 1

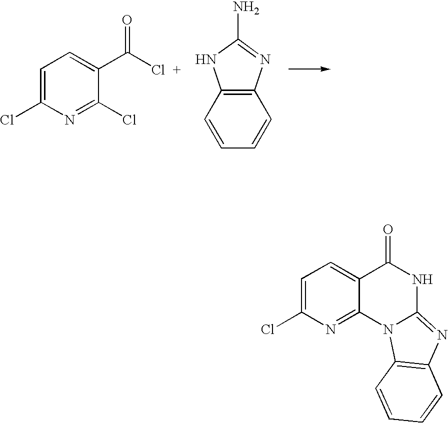

To a suspension of 2,6-dichloronicotinoyl chloride (5.2 g, 24.9 mmol) in methylene chloride (100 mL) was added a solution of 2-aminobenzimidazole (3.3 g, 24.9 mmol, dissolved in 30 mL methylene chloride) dropwise at 0° C. followed by the dropwise addition of triethylamine (7.0 mL, 48.8 mmol). The mixture was allowed to come to room temperature over 1 hour with constant stirring. The solvent was removed in vacuo and replaced with toluene (100 mL) and diisopropylethylamine was added (4.2 mL, 24.9 mmol). The mixture was then heated to on a 125° C. bath for 2 hours then filtered warm. The resulting solid was then stirred in methanol (30 mL) and filtered to afford the cyclized material as a white solid (5.45 g, 20.2 mmol, 81%).

### Example 2

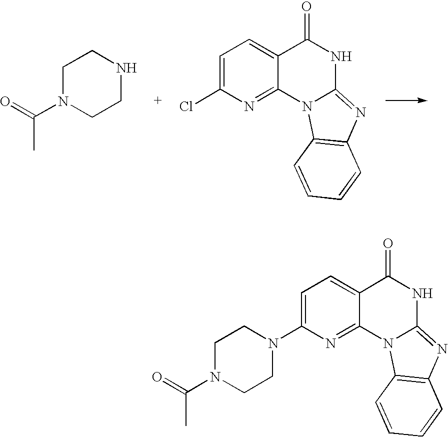

To a suspension of the chloropyridine (1.0 g, 3.7 mmol) in NMP (3.0 mL) was added acetylpiperazine (712 mg, 5.55 mmol) and the mixture was heated on a 100° C. hotplate for 2 hours. The mixture was allowed to cool to room temperature and methanol and water (1:1 mixture, 20 mL) was added. The product was recovered by filtration to afford the amide as a white solid (1.40 g, slightly damp).

### Example 3

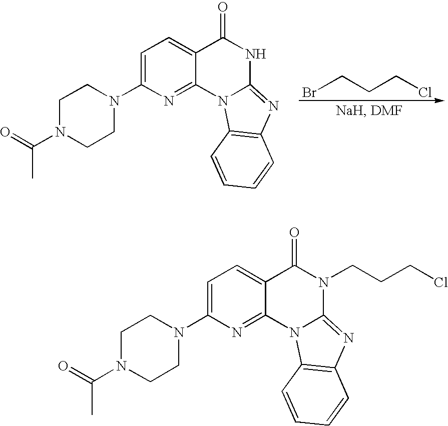

To a suspension of the amide (0.5 g, 1.38 mmol) in DMF (5 mL) was added 1-bromo-3-chloropropane (240 mg, 1.52 mmol) followed by sodium hydride (61 mg, 1.52 mmol) at room temperature and the mixture was allowed to stir overnight. Water was added (20 mL) and the product was collected by filtration, washing with methanol, to afford the chloride as a white solid (287 mg).

### Example 4

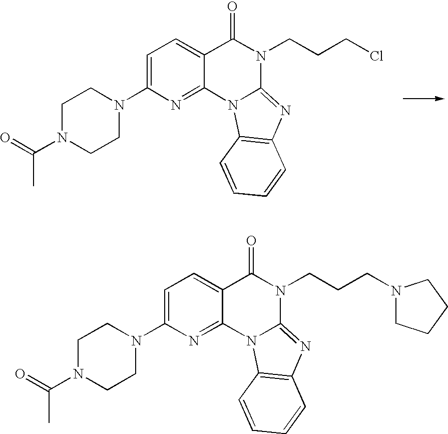

To a solution of the chloride (112 mg) in DMSO (0.5 mL) was added pyrrolidine (large excess) and the mixture was heated to 100° C. for 1 hour. The mixture was allowed to come to room temperature and water was added (20 mL). The product was collected by filtration to afford the pyrrolidine as a white solid (60 mg).

The same methods used to make the compound of Example 4 were followed to synthesize the following compound, except that morpholine was used in place of the piperazine.

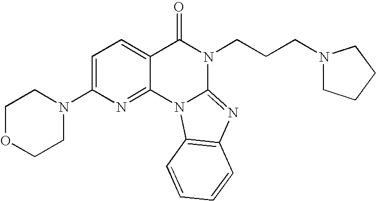

### Example 5

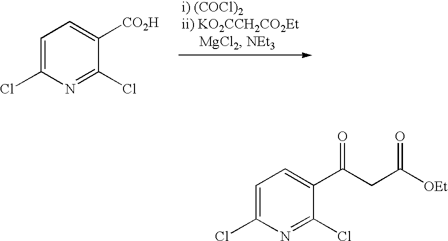

To a solution of the 2,6-dichloronicotinic acid (31.24 g, 162.7 mmol) in methylene chloride (500 mL) was added oxalyl chloride (187.5 mmol, 1.2 eq.) followed by 3 drops of DMF and the mixture was allowed to stir overnight at room temperature. The solvent was then removed in vaccuo to afford the crude acid chloride as oil. In a separate flask was dissolved potassium ethyl malonate (41.5 g, 244 mmol) in acetonitrile (500 mL) and the mixture was cooled to 5° C. Magnesium chloride was then added over 5 minutes, keeping the temperature below 25° C. The crude acid chloride was then dissolved in acetonitrile (50 mL) and was added via dropping funnel keeping the temperature below 5° C. over 30 minutes. Triethylamine was then added as quickly as possible while still keeping the temperature below 10° C. Upon complete addition the reaction was allowed to warm to room temperature overnight with constant stirring. The solvent was removed in vacuo and replaced with ethyl acetate and 1N HCl was added (500 mL) and the mixture was stirred for an additional 30 minutes. The organic layer was separated, washed with brine and dried over sodium sulfate and the solvent was removed in vacuo to afford the ketoester as an orange oil (35.04 g). The product was purified by recrystallization from 10% water/methanol to afford the pure ketoester as a white crystalline solid (31.21 g, 74%). LCMS (ES): 95% pure, elutes as 2 peaks, m/z 216, 262.

### Example 6

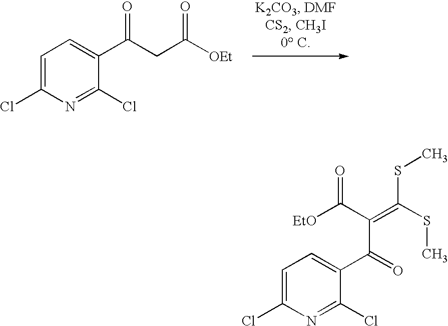

To a solution of the ketoester (11.45 g, 43.87 mmol) was dissolved in DMF (60 mL) and the mixture was chilled to 0° C. with an ice bath. Methyl iodide (8.2 mL, 132.mmol) was then added and the mixture was cooled to −5° C. Carbon disulfide was then added (4.0 mL, 65.8 mmol) followed by potassium carbonate (12.1 g, 88 mmol) keeping the temperature below 5° C. The mixture was allowed to warm to room temperature with stiffing over 2 hours then was extracted with ethyl acetate (5×100 mL) and dried over sodium sulfate. The solvent was removed in vacuo and the resulting oil was purified by silica gel chromatography (10% ethyl acetate/hexanes) to afford the bisthioether as a yellow oil (70%). LCMS (ES): 90% pure, m/z 388 [M+22]⁺, 320 [M+1-OEt]⁺.

### Example 7

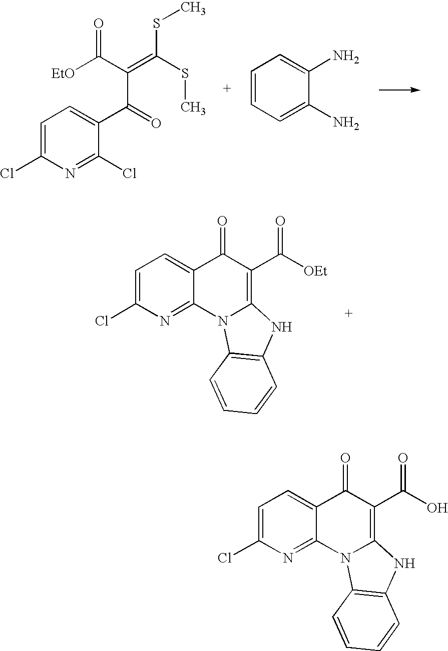

To a solution of the bisthioether (10.14 g, 27.7 mmol) in toluene (300 mL) was added 1,2-phenylenediamine (3.3 g, 30.5 mmol) and the mixture was refluxed overnight with constant argon degassing. The mixture was then allowed to cool to room temperature and diisopropyl ethylamine (7.0 mL, 41.55 mmol) was added and the mixture was heated to reflux for 3 hours. The mixture was allowed to cool to room temperature and the product was collected by filtration to afford the cyclized hydrolyzed acid of as a tan solid (3.5 g g, 11.2 mmol, LCMS (ES): 95% pure, m/z 314 [M+1]⁺. The resulting filtrate was concentrated in vaccuo and triturated with ether (100 mL). The product was collected by filtration to afford the cyclized ester as a tan solid (3.5 g, 10.3 mmol); LCMS (ES): 95% pure, m/z 342 [M+1]⁺.

### Example 8

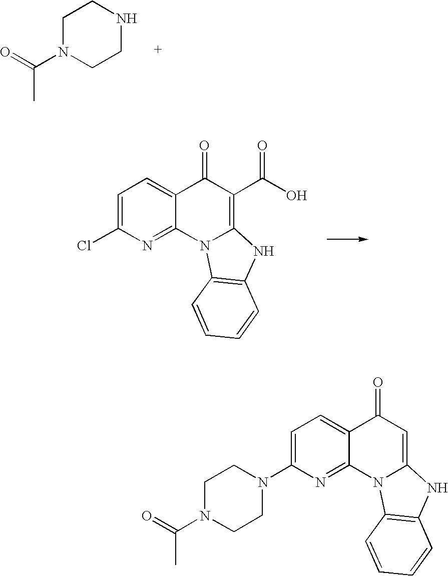

To a solution of the chloroacid (200 mg, 0.64 mmol)) in DMF (2 mL) was added acetyl piperazine (122 mg, 0.96 mmol) and the mixture was heated to 120° C. for 2 hours. The reaction was allowed to cool to room temperature and water was added. The product was collected by filtration to afford the decarboxylated amide as an off white solid (50 mg, 0.14 mmol).

### Example 9

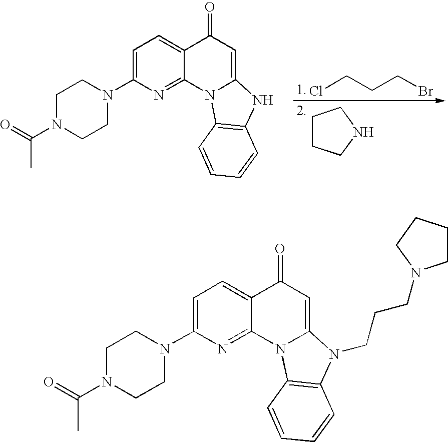

To a solution of the amide (50 mg, 0.14 mmol) in DMF (0.5 mL) was added 1-bromo-3-chloropropane (21 mg, 0.14 mmol) and potassium carbonate (29 mg, 0.21 mmol) and the mixture was heated at 60° C. overnight. The reaction was allowed to come to room temperature and water was added. The resulting solid was filtered and dried and dissolved in DMSO (0.5 mL), pyrrolidine was added (large excess) and the mixture was heated to 100° C. for 2 hours. The crude mixture was purified by mass directed reverse phase chromatography. The solvent was removed in vaccuo and the resulting solid was treated with 2% NH₄OH in methanol and chromatographed on normal phase silica gel (plate) to afford the amine as a white solid (10 mg).

### Example 10

Quadruplex Structures of Ribosmal Nucleic Acids

Circular dichroism (CD) was utilized to determine whether subsequences from ribosomal nucleic acids form quadruplex structures. All sequences were HPLC purified DNA oligonucleotides (sequences 5′ to 3′ as represented hereafter). The name of each sample identifies the approximate location along the rDNA unit as well as the specific strand (NC=non-coding; C=coding). The following procedure was utilized: each oligonucleotide was dissolved at a strand concentration of 5 uM in 200 ul of aqueous buffer containing Tris pH 7.4 (10 mM). The sample was heated to 95° C. for 5 min. then allowed to cool to ambient temperature. CD spectroscopy was performed on a JASCO 810 Spectropolarimeter, using a quartz cell of 1 mm path length. Additional spectra were taken after the addition of 20 ul KCl (1M) to the oligonucleotide solution. Certain compounds have been shown to interact preferentially with a mixed-parallel quadruplex structure in competition assays (e.g., PCT/US2004/033401 filed on Oct. 7, 2004, entitled “Competition Assay for Identifying Modulators of Quadruplex Nucleic Acids”).

Quadruplex structures for other nucleic acids having sequences derived from human ribosomal DNA, template (T) and non-template (NT) strands were tested by the same methods and spectra are summarized in the table below. The nucleic acid identifier notes (i) whether the nucleotide sequence is from the non-template (NT) strand (e.g., SEQ ID NO: 1) or templates (T) strand (e.g., reverse complement of SEQ ID NO: 1) of human rDNA, and the (ii) the location of the sequence in the NT strand or the location in SEQ ID NO: 1 from which the reverse-complement sequence is derived for the T strand of rDNA. For nucleotide sequences from the NT strand, the number in the identifier delineates the 5′ nucleotide of the oligonucleotide and is the position in SEQ ID NO: 1 less one nucleotide (e.g., the nucleotide sequence of oligonucleotide 13079NT spans sixteen (16) nucleotides in SEQ ID NO: 1 beginning at position 13080 in SEQ ID NO: 1). For nucleotide sequences from the T strand, the number in the identifier defines the 3′ nucleotide of the reverse complement oligonucleotide derived from the position in SEQ ID NO: 1 less one nucleotide (e.g., the nucleotide sequence of 10110T is the reverse complement of a seventeen (17) nucleotide span in SEQ ID NO: 1, with the 3′ terminus of the oligonucleotide defined at position 10111 in SEQ ID NO: 1). Spectra characteristic of parallel, mixed parallel, antiparallel (with mixed parallel characteristics) and complex intramolecular quadruplex structures were observed. Quadruplex conformation determinations are summarized in the following table.

| Nucleic | SEQ | | |
|---|---|---|---|
| acid | ID | | |
| identifier | NO. | Conformation | Nucleotide Sequence |
| 10110T | | Parallel | GGGGGGGGGGGCGGGGG |
| 13079NT | | Parallel | GGGGTGGGGGGGAGGG |
| 6960NT | | Mixed | GGGTGGCGGGGGGGAGAGGGGGG |
| 6534NT | | Mixed | GGGCGGGGGGGGCGGGGGG |
| 1196NT | | Mixed | GGGTGGACGGGGGGGCCTGGTGGGG |
| 2957NT | | Mixed | GGGTCGGGGGGTGGGGCCCGGGCCGGGG |
| 5700NT | | Mixed | GGGAGGGAGACGGGGGGG |
| 8511NT | | Mixed | GGGGGTGGGCGGGCGGGGCCGGGGGTGGG |
| 6183NT | | Mixed | GGGTCGGGGGCGGTGGTGGGCCCGCGGGGG |
| 11028NT | | Mixed | GGGGCGCGGCGGGGGGAGAAGGGTCGGGGCGGCAGGGG |
| 6374NT | | Mixed | GGGGGCGGGAACCCCCGGGCGCCTGTGGG |
| 7733T | | Mixed | GGGAGGGGCACGGGCCGGGGGCGGGACGGG |
| 7253NT | | Mixed | GGGTCCGGAAGGGGAAGGGTGCCGGCGGGGAGAGAGGGTCGGGGG |
| 13173NT | | Mixed | GGGCCGGGACGGGGTCCGGGG |
| 6914T | | Mixed | GGGCCCGCGGGGGGAGGGGGAAGGGGCGGG |
| 8749NT | | Antiparallel | GGGAGGGCGCGCGGGTCGGGG |
| 10816NT | | Antiparallel | GGGCTGGGTCGGTCGGGCTGGGG |
| 8762NT | | Complex | CGGAGGGCGCGCGGGTCGGGGCGGCGGCGGCGGCGGCGGTGGCGGCGG |
| | | | CGGCGGGGGCGGCGGG |

### Example 11

### Effects of Ribosomal Nucleic Acid Interacting Molecules on Nucleolin/Nucleic Acid Interactions

The following assays can be used to assess the effects of compounds on interactions between nucleolin and nucleic acid ligands capable of forming quadruplex (QP) and hairpin (HP) secondary structures. Nucleic acid ligands tested were a cMyc QP DNA having nucleotide sequence 5′-TGGGGAGGGTGGGGAGGGTGGGGAAGG-3′ and a HP pre-rRNA region to which nucleolin binds, having the sequence 5′-GGCCGAAAUCCCGAAGUAGGCC-3′. In the assays, recombinant nucleolin (˜250 nM), which has been fused to maltose binding protein, and has the sequence under accession number NM_{—}005381 without the N-terminal acidic stretches domain, is incubated with each of the two ³²P-labeled nucleic acid ligands (10 or 250 nM). Nucleolin and the nucleic acid ligand are incubated in the presence or absence of a test compound 7 in an incubation buffer (12.5 mM Tris, pH 7.6, 60 mM KCl, 1 mM MgCl₂, 0.1 mM EDTA, 1 mM DTT, 5% glycerol, 0.1 mg/ml BSA) for 30 minutes at room temperature.

The resulting complexes are separated on a 6% DNA retardation gel using 0.5×TBE with 20 mM KCl as a running buffer. The assay also can be conducted using nucleic acid ligands derived from human ribosomal DNA, whereby one can identify a compound that selectively modulates formation of a nucleolin/nucleic acid complex that depends on the conformation of the nucleic acid. Sequences of suitable nucleic acids are shown in the preceding example. The table directly below shows for each nucleic acid ligand the relative affinity for nucleolin. A “+” represents the weakest nucleolin affinity and a “++++” represents the strongest nucleolin affinity. The table also shows the conformation of the intramolecular quadruplex structure formed by the nucleic acid ligand determined by circular dichroism, as described above. RND27 is a single-stranded nucleic acid having a random sequence that does not form a quadruplex structure. Using nucleic acids such as these having known conformational properties, one can identify a compound such as the compounds described herein that selectively interferes with binding of nucleolin to a particular quadruplex structure.

| Nucleic acid ligand | Conformation | Affinity for Nucleolin |
|---|---|---|
| 1196NT | Mixed | ++ |
| 2957NT | Mixed | +++ |
| 6183NT | Mixed | + |
| 6374NT | Mixed | − |
| 6534NT | Parallel | +++ |
| 6960NT | Parallel | +++ |
| 7253NT | Mixed | +++ |
| 7733T | Mixed | + |
| 8511NT | Mixed | ++++ |
| 8749NT | Antiparallel | + |
| 8762NT | Complex | ++++ |
| 10816NT | Antiparallel | − |
| 11028NT | Mixed | + |
| 13079NT | Parallel | ++ |
| 13137NT | Mixed | ++ |
| RND27 | Single-stranded | − |

### Example 12

### Inhibition of Protein Kinases

Compounds can also be tested for activity in protein kinase inhibition assays as described herein. All substrates are dissolved and diluted to working stocks in de-ionised water, apart from histone H1 (10× working stock in 20 mM MOPS pH 7.0), PDKtide (10× working stock in 50 mM Tris pH 7.0) ATF2 (which is typically stored at a 20× working stock in 50 mM Tris pH 7.5, 150 mM NaCl, 0.1 mM EGTA, 0.03% Brij-35, 50% glycerol, 1 mM benzamidine, 0.2 mM PMSF and 0.1% R-mercaptoethanol), KKLNRTLSFAEPG and RRRLSFAEPG (50 mM HEPES pH 7.4) and GGEEEEYFELVKKKK (20 mM MOPS pH 7.0). All kinases are pre-diluted to a 10× working concentration prior to addition into the assay. The composition of the dilution buffer for each kinase is detailed below.
1. Blk, c-RAF, CSK, IGF-1R, IR, Lyn, MAPK1, MAPK2, MKK4, MKK6, MKK70, SAPK2a, SAPK2b, SAPK3, SAPK4, Syk, ZAP-70: 50 mM Tris pH 7.5, 0.1 mM EGTA, 0.1 mM Na3VO4, 0.1% beta-mercaptoethanol, 1 mg/ml BSA.
2. JNK1a1, JNK2a2, JNK3, PRK2, ROCK-II: 50 mM Tris pH 7.5, 0.1 mM EGTA, 0.1% beta-mercaptoethanol, 1 mg/ml BSA.
3. PDK1: 50 mM Tris pH 7.5, 0.05% Beta-mercaptoethanol, 1 mg/ml BSA.
4. MEK-1: 25 mM Tris pH 7.5, 0.1 mM EGTA, 0.1% beta-mercaptoethanol, 1 mg/ml BSA.
5. Abl, Abl(T315I), ALK, ALK4, Arg, Ask1, Aurora-A, Axl, Bmx, BRK, BTK, CDK1/cyclinB, CDK2/cyclinA, CDK2/cyclinE, CDK3/cyclinE, CDK5/p25, CDK5/p35, CDK6/cyclinD3, CDK7/cyclinH/MAT1, CHK1, CHK2, CK1, CKIS, cKit, cKit (D816V), cSRC, DDR2, EGFR, EGFR (L858R), EGFR (L861Q), EphA2, EphA3, EphA4, EphA5, EphB2, EphB3, EphB4, ErbB4, Fer, Fes, FGFR1, FGFR2, FGFR3, FGFR4, Fgr, Flt1, Flt3, Flt3 (D835Y), Fms, Fyn, GSK3a, GSK30, Hck, HIPK2, IKKa, IKKO, IRAK4, IRR, JAK2, JAK3, KDR, Lck, MAPKAP-K2, MAPKAP-K3, Met, MINK, MLCK, MRCKP, MSK1, MSK2, MST1, MST2, MuSK, NEK2, NEK6, Nek7, p70S6K, PAK2, PAK-4, PAK6, PAR-1Ba, PDGFRa, PDGFRO, Pim-1, PKA, PKBa, PKBP, PKBy, PKC6, PKCQ, PKG10, Plk3, Pyk2, Ret, RIPK2, Rse, ROCK-I, Ron, Ros, Rsk1, Rsk2, Rsk3, SGK, SGK2, SGK3, Snk, TAK1, TBK1, Tie2, TrkA, TrkB, TSSK2, Yes, ZIPK: 20 mM MOPS pH 7.0, 1 mM EDTA, 0.1% Beta-mercaptoethanol, 0.01% Brij-35, 5% glycerol, 1 mg/ml BSA.
6. CK2: 20 mM HEPES pH 7.6, 0.15 M NaCl, 0.1 mM EGTA, 5 mM DTT, 0.1% Triton X-100, 50% glycerol.
7. CaMKII, CaMKIV: 40 mM HEPES pH 7.4, 1 mg/ml BSA.
8. PKCa, PKCRI, PKCRII, PKCy, PKCS, PKC6, PKCYI, PKCL, PKCμ, PKD2: 20 mM HEPES pH 7.4, 0.03% Triton X-100.
9. PRAK: Beta-mercaptoethanol, 0.1 mM EGTA, 1 mg/ml BSA.
10. AMPK: 50 mM Na R-glycerophosphate pH 7.0, 0.1%.

Protein kinase assays for a variety of kinases are conducted as follows:

Abl (h)

In a final reaction volume of 25 μl, Abl (h) (5-10 mU) is incubated with 8 mM MOPS pH 7.0, 0.2 mM EDTA, 50 μM EAIYAAPFAKKK, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

Abl (T315I) (h)

In a final reaction volume of 25 μl, Abl (T315I) (h) (5-10 mU) is incubated with 8 mM MOPS pH 7.0, 0.2 mM EDTA, 50 μM EAIYAAPFAKKK, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

Abl (m)

In a final reaction volume of 25 μl, Abl (m) (5-10 mU) is incubated with 8 mM MOPS pH 7.0, 0.2 mM EDTA, 50 μM EAIYAAPFAKKK, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted

onto a P30 filtermat and washed three times for 5 minutes in 75 mM phosphoric acid and once in meth\anol prior to drying and scintillation counting.

ALK (h)

In a final reaction volume of 25 μl, ALK (h) (5-10 mU) is incubated with 8 mM MOPS pH 7.0, 0.2 mM EDTA, 250 μM KKKSPGEYVNIEFG, 10 mM MgAcetate and [y-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

ALK4 (h)

In a final reaction volume of 25 μl, ALK4 (h) (5-10 mU) is incubated with 8 mM MOPS pH 7.0, 0.2 mM EDTA,

2 mg/ml casein, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

AMPK (r)

In a final reaction volume of 25 μl, AMPK (r) (5-10 mU) is incubated with 32 mM HEPES pH 7.4, 0.65 mM DTT, 0.012% Brij-35, 200 μM AMP, 200 μM AMARAASAAALARRR, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

Arg (h)

In a final reaction volume of 25 μl, Arg (h) (5-10 mU) is incubated with 8 mM MOPS pH 7.0, 0.2 mM EDTA, 50 μM EAIYAAPFAKKK, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

Arg (m)

In a final reaction volume of 25 μl, Arg (m) (5-10 mU) is incubated with 8 mM MOPS pH 7.0, 0.2 mM EDTA, 50 μM EAIYAAPFAKKK, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted

onto a P30 filtermat and washed three times for 5 minutes in 75 mM phosphoric acid and once in methanol prior to drying and scintillation counting.

ASK1 (h)

In a final reaction volume of 25 μl, ASK1 (h) (5-10 mU) is incubated with 8 mM MOPS pH 7.0, 0.2 mM EDTA, 0.33 mg/ml myelin basic protein, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

Aurora-A (h)

In a final reaction volume of 25 μl, Aurora-A (h) (5-10 mU) is incubated with 8 mM MOPS pH 7.0, 0.2 mM EDTA, 200 μM LRRASLG (Kemptide), 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 50 mM phosphoric acid and once in methanol prior to drying and scintillation counting.

Axl (h)

In a final reaction volume of 25 μl, Axl (h) (5-10 mU) is incubated with 8 mM MOPS pH 7.0, 0.2 mM EDTA, 250 μM KKSRGDYMTMQIG, 10 mM MgAcetate and [y-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

Blk (m)

In a final reaction volume of 25 μl, Blk (m) (5-10 mU) is incubated with 50 mM Tris pH 7.5, 0.1 mM EGTA, 0.1 mM Na3VO4, 0.1% R-mercaptoethanol, 0.1 mg/ml poly(Glu, Tyr) 4:1, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a Filtermat A and washed three times for 5 minutes in 75 mM phosphoric acid and once in methanol prior to drying and scintillation counting.

Bmx (h)

In a final reaction volume of 25 μl, Bmx (h) (5-10 mU) is incubated with 8 mM MOPS pH 7.0, 0.2 mM EDTA, 0.1 mg/ml poly(Glu, Tyr) 4:1, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a Filtermat A and washed three times for 5 minutes in 75 mM phosphoric acid and once in methanol prior to drying and scintillation counting.

BRK (h)

In a final reaction volume of 25 μl, BRK (h) (5-10 mU) is incubated with 8 mM MOPS pH 7.0, 0.2 mM EDTA, 5 mM MnCl₂, 0.1 mg/ml poly (Glu, Tyr) 4:1, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a Filtermat A and washed three times for 5 minutes in 75 mM phosphoric acid and once in methanol prior to drying and scintillation counting.

BTK (h)

In a final reaction volume of 25 μl, BTK (h) (5-10 mU) is incubated with 8 mM MOPS pH 7.0, 0.2 mM EDTA, 250 μM KVEKIGEGTYGVVYK (Cdc2 peptide), 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

CaMKII (r)

In a final reaction volume of 25 μl, CaMKII (r) (5-10 mU) is incubated with 40 mM HEPES pH 7.4, 5 mM CaCl₂, 30 μg/ml calmodulin, 30 μM KKLNRTLSVA, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

CaMKIV (h)

In a final reaction volume of 25 μl, CaMKIV (h) (5-10 mU) is incubated with 40 mM HEPES pH 7.4, 5 mM CaCl₂, 30 μg/ml calmodulin, 30 μM KKLNRTLSVA, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

CDK1/cyclinB (h)

In a final reaction volume of 25 μl, CDK1/cyclinB (h) (5-10 mU) is incubated with 8 mM MOPS pH 7.0, 0.2 mM EDTA, 0.1 mg/ml histone H1, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

CDK2/cyclinA (h)

In a final reaction volume of 25 μl, CDK2/cyclinA (h) (5-10 mU) is incubated with 8 mM MOPS pH 7.0, 0.2 mM EDTA, 0.1 mg/ml histone H1, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

CDK2/cyclinE (h)

In a final reaction volume of 25 μl, CDK2/cyclinE (h) (5-10 mU) is incubated with 8 mM MOPS pH 7.0, 0.2 mM EDTA, 0.1 mg/ml histone H1, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

CDK3/cyclinE (h)

In a final reaction volume of 25 μl, CDK3/cyclinE (h) (5-10 mU) is incubated with 8 mM MOPS pH 7.0, 0.2 mM EDTA, 0.1 mg/ml histone H1, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

CDK5/p25 (h)

In a final reaction volume of 25 μl, CDK5/p25 (h) (5-10 mU) is incubated with 8 mM MOPS pH 7.0, 0.2 mM EDTA, 0.1 mg/ml histone H1, 10 mM MgAcetate and [y-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

CDK5/p35 (h)

In a final reaction volume of 25 μl, CDK5/p35 (h) (5-10 mU) is incubated with 8 mM MOPS pH 7.0, 0.2 mM EDTA, 0.1 mg/ml histone H1, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

CDK6/cyclinD3 (h)

In a final reaction volume of 25 μl, CDK6/cyclinD3 (h) (5-10 mU) is incubated with 8 mM MOPS pH 7.0, 0.2 mM EDTA, 0.1 mg/ml histone H1, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

CDK7/cyclinH/MAT1 (h)

In a final reaction volume of 25 μl, CDK7/cyclinH/MAT1 (h) (5-10 mU) is incubated with 8 mM MOPS pH 7.0, 0.2 mM EDTA, 500 μM peptide, 10 mM MgAcetate and [y-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

CHK1 (h)

In a final reaction volume of 25 μl, CHK1 (h) (5-10 mU) is incubated with 8 mM MOPS pH 7.0, 0.2 mM EDTA, 200 μM KKKVSRSGLYRSPSMPENLNRPR, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

CHK2 (h)

In a final reaction volume of 25 μl, CHK2 (h) (5-10 mU) is incubated with 8 mM MOPS pH 7.0, 0.2 mM EDTA, 200 μM KKKVSRSGLYRSPSMPENLNRPR, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

CK1 (y)

In a final reaction volume of 25 μl, CK1 (y) (5-10 mU) is incubated with 8 mM MOPS pH 7.0, 0.2 mM EDTA, 200 μM KRRRALS(p)VASLPGL, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

CK1S (h)

In a final reaction volume of 25 μl, CK1S (h) (5-10 mU) is incubated with 8 mM MOPS pH 7.0, 0.2 mM EDTA, 200 μM KRRRALS(p)VASLPGL, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

CK2 (h)

In a final reaction volume of 25 μl, CK2 (h) (5-10 mU) is incubated with 20 mM HEPES pH 7.6, 0.15 M NaCl, 0.1 mM EDTA, 5 mM DTT, 0.1% Triton X-100, 165 μM RRRDDDSDDD, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

cKit (h)

In a final reaction volume of 25 μl, cKit (h) (5-10 mU) is incubated with 8 mM MOPS pH 7.0, 0.2 mM EDTA, 10 mM MnCl₂, 0.1 mg/ml poly(Glu, Tyr) 4:1, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a Filtermat A and washed three times for 5 minutes in 75 mM phosphoric acid and once in methanol prior to drying and scintillation counting.

cKit (D 816V) (h)

In a final reaction volume of 25 μl, cKit (D816V) (h) (5-10 mU) is incubated with 8 mM MOPS pH 7.0, 0.2 mM EDTA, 10 mM MnCl₂, 0.1 mg/ml poly(Glu, Tyr) 4:1, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a Filtermat A and washed three times for 5 minutes in 75 mM phosphoric acid and once in methanol prior to drying and scintillation counting.

c-RAF (h)

In a final reaction volume of 25 μl, c-RAF (h) (5-10 mU) is incubated with 25 mM Tris pH 7.5, 0.02 mM EGTA, 0.66 mg/ml myelin basic protein, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

CSK (h)

In a final reaction volume of 25 μl, CSK (h) (5-10 mU) is incubated with 50 mM Tris pH 7.5, 0.1 mM EGTA, 0.1 mM Na3VO4, 0.1% R-mercaptoethanol, 0.1 mg/ml poly(Glu, Tyr) 4:1, 10 mM MnCl₂, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a Filtermat A and washed three times for 5 minutes in 75 mM phosphoric acid and once in methanol prior to drying and scintillation counting.

cSRC (h)

In a final reaction volume of 25 μl, cSRC (h) (5-10 mU) is incubated with 8 mM MOPS pH 7.0, 0.2 mM EDTA, 250 μM KVEKIGEGTYGVVYK (Cdc2 peptide), 10 mM MgAcetate and [y-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

DDR2 (h)

In a final reaction volume of 25 μl, DDR2 (h) (5-10 mU) is incubated with 8 mM MOPS pH 7.0, 0.2 mM EDTA, 250 μM KKSRGDYMTMQIG, 10 mM MnCl₂, 10 mM MgAcetate and [y-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

EGFR (h)

In a final reaction volume of 25 μl, EGFR (h) (5-10 mU) is incubated with 8 mM MOPS pH 7.0, 0.2 mM EDTA, 10 mM MnCl₂, 0.1 mg/ml poly(Glu, Tyr) 4:1, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a Filtermat A and washed three times for 5 minutes in 75 mM phosphoric acid and once in methanol prior to drying and scintillation counting.

EGFR (L858R) (h)

In a final reaction volume of 25 μl, EGFR (L858R) (h) (5-10 mU) is incubated with 8 mM MOPS pH 7.0, 0.2 mM EDTA, 0.1 mg/ml poly(Glu, Tyr) 4:1, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a Filtermat A and washed three times for 5 minutes in 75 mM phosphoric acid and once in methanol prior to drying and scintillation counting.

EGFR (L861Q) (h)

In a final reaction volume of 25 μl, EGFR (L861Q) (h) (5-10 mU) is incubated with 8 mM MOPS pH 7.0, 0.2 mM EDTA, 0.1 mg/ml poly(Glu, Tyr) 4:1, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a Filtermat A and washed three times for 5 minutes in 75 mM phosphoric acid and once in methanol prior to drying and scintillation counting.

EphA2 (h)

In a final reaction volume of 25 μl, EphA2 (h) (5-10 mU) is incubated with 8 mM MOPS pH 7.0, 0.2 mM EDTA,

0.1 mg/ml poly(Glu, Tyr) 4:1, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a Filtermat A and washed three times for 5 minutes in 75 mM phosphoric acid and once in methanol prior to drying and scintillation counting.

EphA3 (h)

In a final reaction volume of 25 μl, EphA3 (h) (5-10 mU) is incubated with 8 mM MOPS pH 7.0, 0.2 mM EDTA,

0.1 mg/ml poly(Glu, Tyr) 4:1, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a Filtermat

A and washed three times for 5 minutes in 75 mM phosphoric acid and once in methanol prior to drying and scintillation counting.

EphA4 (h)

In a final reaction volume of 25 μl, EphA4 (h) (5-10 mU) is incubated with 8 mM MOPS pH 7.0, 0.2 mM EDTA,

10 mM MnCl₂, 0.1 mg/ml poly(Glu, Tyr) 4:1, 10 mM MgAcetate and [y-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a Filtermat A and washed three times for 5 minutes in 75 mM phosphoric acid and

once in methanol prior to drying and scintillation counting.

EphA5 (h)

In a final reaction volume of 25 μl, EphA5 (h) (5-10 mU) is incubated with 8 mM MOPS pH 7.0, 0.2 mM EDTA, 2.5 mM MnCl₂, 0.1 mg/ml poly (Glu, Tyr) 4:1, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a Filtermat A and washed three times for 5 minutes in 75 mM phosphoric acid and once in methanol prior to drying and scintillation counting.

EphB2 (h)

In a final reaction volume of 25 μl, EphB2 (h) (5-10 mU) is incubated with 8 mM MOPS pH 7.0, 0.2 mM EDTA, 10 mM MnCl₂, 0.1 mg/ml poly(Glu, Tyr) 4:1, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a Filtermat A and washed three times for 5 minutes in 75 mM phosphoric acid and once in methanol prior to drying and scintillation counting.

EphB3 (h)

In a final reaction volume of 25 μl, EphB3 (h) (5-10 mU) is incubated with 8 mM MOPS pH 7.0, 0.2 mM EDTA, 10 mM MnCl₂, 0.1 mg/ml poly (Glu, Tyr) 4:1, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a Filtermat A and washed three times for 5 minutes in 75 mM phosphoric acid and once in methanol prior to drying and scintillation counting.

EphB4 (h)

In a final reaction volume of 25 μl, EphB4 (h) (5-10 mU) is incubated with 8 mM MOPS pH 7.0, 0.2 mM EDTA, 10 mM MnCl₂, 0.1 mg/ml poly(Glu, Tyr) 4:1, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a Filtermat A and washed three times for 5 minutes in 75 mM phosphoric acid and once in methanol prior to drying and scintillation counting.

ErbB4 (h)

In a final reaction volume of 25 μl, ErbB4 (h) (5-10 mU) is incubated with 8 mM MOPS pH 7.0, 0.2 mM EDTA, 2.5 mM MnCl₂, 0.1 mg/ml poly(Glu, Tyr) 4:1, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a Filtermat A and washed three times for 5 minutes in 75 mM phosphoric acid and once in methanol prior to drying and scintillation counting.

Fer (h)

In a final reaction volume of 25 μl, Fer (h) (5-10 mU) is incubated with 8 mM MOPS pH 7.0, 0.2 mM EDTA, 1 mM MnCl₂, 250 μM KKKSPGEYVNIEFG, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

Fes (h)

In a final reaction volume of 25 μl, Fes (h) (5-10 mU) is incubated with 8 mM MOPS pH 7.0, 0.2 mM EDTA, 0.1 mg/ml poly(Glu, Tyr) 4:1, 10 mM MgAcetate and [y-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted

onto a Filtermat A and washed three times for 5 minutes in 75 mM phosphoric acid and once in methanol prior to drying and scintillation counting.

FGFR1 (h)

In a final reaction volume of 25 μl, FGFR1 (h) (5-10 mU) is incubated with 8 mM MOPS pH 7.0, 0.2 mM EDTA, 250 μM KKKSPGEYVNIEFG, 10 mM MgAcetate and [y-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

FGFR2 (h)

In a final reaction volume of 25 μl, FGFR2 (h) (5-10 mU) is incubated with 8 mM MOPS pH 7.0, 0.2 mM EDTA, 2.5 mM MnCl₂, 0.1 mg/ml poly(Glu, Tyr) 4:1, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a Filtermat A and washed three times for 5 minutes in 75 mM phosphoric acid and once in methanol prior to drying and scintillation counting.

FGFR3 (h)

In a final reaction volume of 25 μl, FGFR3 (h) (5-10 mU) is incubated with 8 mM MOPS pH 7.0, 0.2 mM EDTA, 0.1 mg/ml poly(Glu, Tyr) 4:1, 10 mM MnCl₂, 10 mM MgAcetate and [y-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a Filtermat A and washed three times for 5 minutes in 75 mM phosphoric acid and once in methanol prior to drying and scintillation counting.

FGFR4 (h)

In a final reaction volume of 25 μl, FGFR4 (h) (5-10 mU) is incubated with 8 mM MOPS pH 7.0, 0.2 mM EDTA, 10 mM MnCl₂, 0.1 mg/ml poly(Glu, Tyr) 4:1, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a Filtermat A and washed three times for 5 minutes in 75 mM phosphoric acid and once in methanol prior to drying and scintillation counting.

Fgr (h)

In a final reaction volume of 25 μl, Fgr (h) (5-10 mU) is incubated with 8 mM MOPS pH 7.0, 0.2 mM EDTA, 0.1 mg/ml poly(Glu, Tyr) 4:1, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a Filtermat A and washed three times for 5 minutes in 75 mM phosphoric acid and once in methanol prior to drying and scintillation counting.

Flt1 (h)

In a final reaction volume of 25 μl, Flt1 (h) (5-10 mU) is incubated with 8 mM MOPS pH 7.0, 0.2 mM EDTA,

250 μM KKKSPGEYVNIEFG, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

Flt3 (h)

In a final reaction volume of 25 μl, Flt3 (h) (5-10 mU) is incubated with 8 mM MOPS pH 7.0, 0.2 mM EDTA, 50 μM EAIYAAPFAKKK, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

Flt3 (D835Y) (h)

In a final reaction volume of 25 μl, Flt3 (D835Y) (h) (5-10 mU) is incubated with 8 mM MOPS pH 7.0, 0.2 mM EDTA, 50 μM EAIYAAPFAKKK, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

Fms (h)

In a final reaction volume of 25 μl, Fms (h) (5-10 mU) is incubated with 8 mM MOPS pH 7.0, 0.2 mM EDTA, 250 μM KKKSPGEYVNIEFG, 10 mM MgAcetate and [y-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

Fyn (h)

In a final reaction volume of 25 μl, Fyn (h) (5-10 mU) is incubated with 50 mM Tris pH 7.5, 0.1 mM EGTA, 0.1 mM Na3VO4, 250 μM KVEKIGEGTYGVVYK (Cdc2 peptide), 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

GSK3a (h)

In a final reaction volume of 25 μl, GSK3a (h) (5-10 mU) is incubated with 8 mM MOPS pH 7.0, 0.2 mM EDTA, 20 μM YRRAAVPPSPSLSRHSSPHQS(p)EDEEE (phospho GS2 peptide), 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 50 mM phosphoric acid and once in methanol prior to drying and scintillation counting.

GSK3P (h)

In a final reaction volume of 25 μl, GSK30 (h) (5-10 mU) is incubated with 8 mM MOPS pH 7.0, 0.2 mM EDTA, 20 μM YRRAAVPPSPSLSRHSSPHQS(p)EDEEE (phospho GS2 peptide), 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 50 mM phosphoric acid and once in methanol prior to drying and scintillation counting.

Hck (h)

In a final reaction volume of 25 μl, Hck (h) (5-10 mU) is incubated with 8 mM MOPS pH 7.0, 0.2 mM EDTA,

250 μM KVEKIGEGTYGVVYK (Cdc2 peptide), 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

HIPK2 (h)

In a final reaction volume of 25 μl, HIPK2 (h) (5-10 mU) is incubated with 8 mM MOPS pH 7.0, 0.2 mM EDTA, 0.33 mg/ml myelin basic protein, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

IGF-1R (h)

In a final reaction volume of 25 μl, IGF-1R (h) (5-10 mU) is incubated with 50 mM Tris pH 7.5, 0.1 mM EGTA, 0.1 mM Na3VO4, 0.1% R-mercaptoethanol, 250 μM KKKSPGEYVNIEFG, 10 mM MnCl₂, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

IKKa (h)

In a final reaction volume of 25 μl, IKKa (h) (5-10 mU) is incubated with 8 mM MOPS pH 7.0, 0.2 mM EDTA, 200 μM peptide, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

IKKP (h)

In a final reaction volume of 25 μl, IKKP (h) (5-10 mU) is incubated with 8 mM MOPS pH 7.0, 0.2 mM EDTA, 100 μM peptide, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

IR (h)

In a final reaction volume of 25 μl, IR (h) (5-10 mU) is incubated with 50 mM Tris pH 7.5, 0.1 mM EGTA, 0.1 mM Na3VO4, 0.1% R-mercaptoethanol, 250 μM KKSRGDYMTMQIG, 10 mM MnCl₂, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

IRAK4 (h)

In a final reaction volume of 25 μl, IRAK4 (h) (5-10 mU) is incubated with 8 mM MOPS pH 7.0, 0.2 mM EDTA,

0.33 mg/ml myelin basic protein, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

IRR (h)

In a final reaction volume of 25 μl, IRR (h) (5-10 mU) is incubated with 8 mM MOPS pH 7.0, 0.2 mM EDTA, 0.33 mg/ml myelin basic protein, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

JAK2 (h)

In a final reaction volume of 25 μl, JAK2 (h) (5-10 mU) is incubated with 8 mM MOPS pH 7.0, 0.2 mM EDTA, 10011M KTFCGTPEYLAPEVRREPRILSEEEQEMFRDFDYIADWC, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

JAK3 (h)

In a final reaction volume of 25 μl, JAK3 (h) (5-10 mU) is incubated with 8 mM MOPS pH 7.0, 0.2 mM EDTA, 500 μM GGEEEEYFELVKKKK, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

JNK1a1 (h)

In a final reaction volume of 25 μl, JNK1a1 (h) (5-10 mU) is incubated with 50 mM Tris pH 7.5, 0.1 mM EGTA, 0.1% R-mercaptoethanol, 3 μM ATF2, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

JNK2a2 (h)

In a final reaction volume of 25 μl, JNK2a2 (h) (5-10 mU) is incubated with 50 mM Tris pH 7.5, 0.1 mM EGTA, 0.1% R-mercaptoethanol, 3 μM ATF2, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

JNK3 (h)

In a final reaction volume of 25 μl, JNK3 (h) (5-10 mU) is incubated with 50 mM Tris pH 7.5, 0.1 mM EGTA, 0.1% R-mercaptoethanol, 250 μM peptide, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

KDR (h)

In a final reaction volume of 25 μl, KDR (h) (5-10 mU) is incubated with 8 mM MOPS pH 7.0, 0.2 mM EDTA,

0.33 mg/ml myelin basic protein, 10 mM MgAcetate and [y-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

Lck (h)

In a final reaction volume of 25 μl, Lck (h) (5-10 mU) is incubated with 50 mM Tris pH 7.5, 0.1 mM EGTA, 0.1 mM Na3VO4, 250 μM KVEKIGEGTYGVVYK (Cdc2 peptide), 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

Lyn (h)

In a final reaction volume of 25 μl, Lyn (h) (5-10 mU) is incubated with 50 mM Tris pH 7.5, 0.1 mM EGTA, 0.1 mM Na3VO4, 0.1% R-mercaptoethanol, 0.1 mg/ml poly(Glu, Tyr) 4:1, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a Filtermat A and washed three times for 5 minutes in 75 mM phosphoric acid and once in methanol prior to drying and scintillation counting.

Lyn (m)

In a final reaction volume of 25 μl, Lyn (m) (5-10 mU) is incubated with 50 mM Tris pH 7.5, 0.1 mM EGTA, 0.1 mM Na3VO4, 0.1% R-mercaptoethanol, 0.1 mg/ml poly(Glu, Tyr) 4:1, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a Filtermat A and washed three times for 5 minutes in 75 mM phosphoric acid and once in methanol prior to drying and scintillation counting.

MAPK1 (h)

In a final reaction volume of 25 μl, MAPK1 (h) (5-10 mU) is incubated with 25 mM Tris pH 7.5, 0.02 mM EGTA, 250 μM peptide, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

MAPK2 (h)

In a final reaction volume of 25 μl, MAPK2 (h) (5-10 mU) is incubated with 25 mM Tris pH 7.5, 0.02 mM EGTA, 0.33 mg/ml myelin basic protein, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

P,-MAPK2 (m)

In a final reaction volume of 25 μl, MAPK2 (m) (5-10 mU) is incubated with 25 mM Tris pH 7.5, 0.02 mM EGTA, 0.33 mg/ml myelin basic protein, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

MAPKAP-K2 (h)

In a final reaction volume of 25 μl, MAPKAP-K2 (h) (5-10 mU) is incubated with 50 mM Na R-glycerophosphate pH 7.5, 0.1 mM EGTA, 30 μM KKLNRTLSVA, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

MAPKAP-K3 (h)

In a final reaction volume of 25 μl, MAPKAP-K3 (h) (5-10 mU) is incubated with 50 mM Na R-glycerophosphate pH 7.5, 0.1 mM EGTA, 30 μM KKLNRTLSVA, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

MEK1 (h)

In a final reaction volume of 25 μl, MEK1 (h) (1-5 mU) is incubated with 50 mM Tris pH 7.5, 0.2 mM EGTA, 0.1% R-mercaptoethanol, 0.01% Brij-35, 1 μM inactive MAPK2 (m), 10 mM MgAcetate and cold ATP (concentration as required). The reaction is initiated by the addition of the MgATP. After incubation for 40 minutes at room temperature, 5 μl of this incubation mix is used to initiate a MAPK2 (m) assay, which is described on page 12 of this book.

Met (h)

In a final reaction volume of 25 μl, Met (h) (5-10 mU) is incubated with 8 mM MOPS pH 7.0, 0.2 mM EDTA, 250 μM KKKSPGEYVNIEFG, 10 mM MgAcetate and [y-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

MINK (h)

In a final reaction volume of 25 μl, MINK (h) (5-10 mU) is incubated with 8 mM MOPS pH 7.0, 0.2 mM EDTA, 0.33 mg/ml myelin basic protein, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

MKK4 (m)

In a final reaction volume of 25 μl, MKK4 (m) (1-5 mU) is incubated with 50 mM Tris pH 7.5, 0.1 mM EGTA, 0.1% R-mercaptoethanol, 0.1 mM Na3VO4, 2 μM inactive JNK1a1 (h), 10 mM MgAcetate and cold ATP (concentration as required). The reaction is initiated by the addition of the MgATP. After incubation for 40 minutes at room temperature, 5 μl of this incubation mix is used to initiate a JNK1a1 (h) assay, which is exactly as described on page 11 of this book except that ATF2 is replaced with 250 μM peptide.

MKK6 (h)

In a final reaction volume of 25 μl, MKK6 (h) (1-5 mU) is incubated with 50 mM Tris pH 7.5, 0.1 mM EGTA, 0.1% R-mercaptoethanol, 0.1 mM Na3VO4, 1 mg/ml BSA, 1 μM inactive SAPK2a (h), 10 mM MgAcetate and cold ATP (concentration as required). The reaction is initiated by the addition of the MgATP. After incubation for 40 minutes at room temperature, 5 μl of this incubation mix is used to initiate a SAPK2a (h) assay, which is described on page 18 of this book.

MKK7P (h)

In a final reaction volume of 25 μl, MKK70 (h) (1-5 mU) is incubated with 50 mM Tris pH 7.5, 0.1 mM EGTA, 0.1% R-mercaptoethanol, 0.1 mM Na3VO4, 211M inactive JNK1a1 (h), 10 mM MgAcetate and cold ATP (concentration as required). The reaction is initiated by the addition of the MgATP. After incubation for 40 minutes at room temperature, 5 μl of this incubation mix is used to initiate a JNK1a1 (h) assay, which is exactly as described on page 11 of this book except that ATF2 is replaced with 250 μM peptide.

MLCK (h)

In a final reaction volume of 25 μl, MLCK (h) (5-10 mU) is incubated with 8 mM MOPS pH 7.0, 0.2 mM EDTA, 0.5 mM CaCl₂, 16 μg/ml calmodulin, 250 μM KKLNRTLSFAEPG, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

MRCKP (h)

In a final reaction volume of 25 μl, MRCKP (h) (5-10 mU) is incubated with 8 mM MOPS pH 7.0, 0.2 mM EDTA, 100 μM KKRNRTLTV, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

MSK1 (h)

In a final reaction volume of 25 μl, MSK1 (h) (5-10 mU) is incubated with 8 mM MOPS pH 7.0, 0.2 mM EDTA,

30 μM GRPRTSSFAEGKK, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

MSK2 (h)

In a final reaction volume of 25 μl, MSK2 (h) (5-10 mU) is incubated with 8 mM MOPS pH 7.0, 0.2 mM EDTA, 30 μM GRPRTSSFAEGKK, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted

onto a P30 filtermat and washed three times for 5 minutes in 75 mM phosphoric acid and once in methanol prior to drying and scintillation counting.

MST1 (h)

In a final reaction volume of 25 μl, MST1 (h) (5-10 mU) is incubated with 8 mM MOPS pH 7.0, 0.2 mM EDTA, 250 μM KKSRGDYMTMQIG, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted

onto a P30 filtermat and washed three times for 5 minutes in 75 mM phosphoric acid and once in methanol prior to drying and scintillation counting.

MST2 (h)

In a final reaction volume of 25 μl, MST2 (h) (5-10 mU) is incubated with 8 mM MOPS pH 7.0, 0.2 mM EDTA, 0.33 mg/ml myelin basic protein, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

MuSK (h)

In a final reaction volume of 25 μl, MuSK (h) (5-10 mU) is incubated with 8 mM MOPS pH 7.0, 0.2 mM EDTA, 5 mM MnCl₂, 0.33 mg/ml myelin basic protein, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

NEK2 (h)

In a final reaction volume of 25 μl, NEK2 (h) (5-10 mU) is incubated with 8 mM MOPS pH 7.0, 0.2 mM EDTA, 0.33 mg/ml myelin basic protein, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

NEK6 (h)

In a final reaction volume of 25 μl, NEK6 (h) (5-10 mU) is incubated with 8 mM MOPS pH 7.0, 0.2 mM EDTA, 300 μM FLAKSFGSPNRAYKK, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

NEK7 (h)

In a final reaction volume of 25 μl, NEK7 (h) (5-10 mU) is incubated with 8 mM MOPS pH 7.0, 0.2 mM EDTA, 300 μM FLAKSFGSPNRAYKK, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

PAK2 (h)

In a final reaction volume of 25 μl, PAK2 (h) (5-10 mU) is incubated with 8 mM MOPS pH 7.0, 0.2 mM EDTA, 30 μM KEAKEKRQEQIAKRRRLSSLRASTSKSGGSQK, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

PAK-4 (h)

In a final reaction volume of 25 μl, PAK-4 (h) (5-10 mU) is incubated with 8 mM MOPS pH 7.0, 0.2 mM EDTA, 0.8 mg/ml myelin basic protein, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

PAK6 (h)

In a final reaction volume of 25 μl, PAK6 (h) (5-10 mU) is incubated with 8 mM MOPS pH 7.0, 0.2 mM EDTA, 200 μM RRRLSFAEPG, 10 mM MgAcetate and [y-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted

onto a P30 filtermat and washed three times for 5 minutes in 75 mM phosphoric acid and once in methanol prior to drying and scintillation counting.

PAR-1B a (h)

In a final reaction volume of 25 μl, PAR-1Ba (h) (5-10 mU) is incubated with 8 mM MOPS pH 7.0, 0.2 mM EDTA, 10011M KKKVSRSGLYRSPSMPENLNRPR, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

PDGFRa (h)

In a final reaction volume of 25 μl, PDGFRa (h) (5-10 mU) is incubated with 8 mM MOPS pH 7.0, 0.2 mM EDTA, 0.1 mg/ml poly(Glu, Tyr) 4:1, 10 mM MnCl₂, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a Filtermat A and washed three times for 5 minutes in 75 mM phosphoric acid and once in methanol prior to drying and scintillation counting.

PDGFRP (h)

In a final reaction volume of 25 μl, PDGFRP (h) (5-10 mU) is incubated with 8 mM MOPS pH 7.0, 0.2 mM EDTA, 0.1 mg/ml poly(Glu, Tyr) 4:1, 10 mM MnCl2, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a Filtermat A and washed three times for 5 minutes in 75 mM phosphoric acid and once in methanol prior to drying and scintillation counting.

PDK1 (h)

In a final reaction volume of 25 μl, PDK1 (h) (5-10 mU) is incubated with 50 mM Tris pH 7.5, 100 μM KTFCGTPEYLAPEVRREPRILSEEEQEMFRDFDYIADWC(PDKtide), 0.1% R-mercaptoethanol, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

PI3Ky (h) [Non-Radioactive Assay]

In a final reaction volume of 20 μl, PI3Ky (h) is incubated in assay buffer containing 10 μM phosphatidylinositol-4,5-bisphosphate and MgATP (concentration as required). The reaction is initiated by the addition of the MgATP mix. After incubation for 30 minutes at room temperature, the reaction is stopped by the addition of 5 μl of stop solution containing EDTA and biotinylated phosphatidylinositol-3,4,5-trisphosphate. Finally, 5 μl of detection buffer is added, which contains europium-labelled anti-GST monoclonal antibody, GST-tagged GRP1 PH domain and streptavidin-allophycocyanin. The plate is then read in time-resolved fluorescence mode and the homogenous time-resolved fluorescence (HTRF®)*signal is determined according to the formula HTRF®=10000×(Em665 nm/Em620 nm).

Pim-1 (h)

In a final reaction volume of 25 μl, Pim-1 (h) (5-10 mU) is incubated with 8 mM MOPS pH 7.0, 0.2 mM EDTA, 10011M KKRNRTLTV, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

PKA (h)

In a final reaction volume of 25 μl, PKA (h) (5-10 mU) is incubated with 8 mM MOPS pH 7.0, 0.2 mM EDTA, 30 μM LRRASLG (Kemptide), 10 mM MgAcetate and [y-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 50 mM phosphoric acid and once in methanol prior to drying and scintillation counting.

PKA (b)

In a final reaction volume of 25 μl, PKA (b) (5-10 mU) is incubated with 8 mM MOPS pH 7.0, 0.2 mM EDTA, 30 μM LRRASLG (Kemptide), 10 mM MgAcetate and [y-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 50 mM phosphoric acid and once in methanol prior to drying and scintillation counting.

PKBa (h)

In a final reaction volume of 25 μl, PKBa (h) (5-10 mU) is incubated with 8 mM MOPS pH 7.0, 0.2 mM EDTA, 30 μM GRPRTSSFAEGKK, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

PKBP (h)

In a final reaction volume of 25 μl, PKBP (h) (5-10 mU) is incubated with 8 mM MOPS pH 7.0, 0.2 mM EDTA, 30 μM GRPRTSSFAEGKK, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted

onto a P30 filtermat and washed three times for 5 minutes in 75 mM phosphoric acid and once in methanol prior to drying and scintillation counting.

PKBy (h)

In a final reaction volume of 25 μl, PKBy (h) (5-10 mU) is incubated with 8 mM MOPS pH 7.0, 0.2 mM EDTA, 30 μM GRPRTSSFAEGKK, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

PKCa (h)

In a final reaction volume of 25 μl, PKCa (h) (5-10 mU) is incubated with 20 mM HEPES pH 7.4, 0.03% Triton X-100, 0.1 mM, 0.1 mg/ml phosphatidylserine, 10 μg/ml diacylglycerol, 0.1 mg/ml histone H1, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

PKCPI (h)

In a final reaction volume of 25 μl, PKCRI (h) (5-10 mU) is incubated with 20 mM HEPES pH 7.4, 0.03% Triton X-100, 0.1 mM CaCl₂, 0.1 mg/ml phosphatidylserine, 10 μg/ml diacylglycerol, 0.1 mg/ml histone H1, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

PKCPII (h)

In a final reaction volume of 25 μl, PKCRII (h) (5-10 mU) is incubated with 20 mM HEPES pH 7.4, 0.03% Triton X-100, 0.1 mM, 0.1 mg/ml phosphatidylserine, 10 μg/ml diacylglycerol, 0.1 mg/ml histone H1, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

PKCy (h)

In a final reaction volume of 25 μl, PKCy (h) (5-10 mU) is incubated with 20 mM HEPES pH 7.4, 0.03% Triton X-100, 0.1 mM, 0.1 mg/ml phosphatidylserine, 10 μg/ml diacylglycerol, 0.1 mg/ml histone H1, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

PKCS (h)

In a final reaction volume of 25 μl, PKCS (h) (5-10 mU) is incubated with 20 mM HEPES pH 7.4, 0.03% Triton X-100, 0.1 mg/ml phosphatidylserine, 10 μg/ml diacylglycerol, 50 μM ERMRPRKRQGSVRRRV, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

PKCS (h)

In a final reaction volume of 25 μl, PKC6 (h) (5-10 mU) is incubated with 20 mM HEPES pH 7.4, 0.03% Triton X-100, 0.1 mg/ml phosphatidylserine, 10 μg/ml diacylglycerol, 50 μM ERMRPRKRQGSVRRRV, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

PKCYI (h)

In a final reaction volume of 25 μl, PKCYj (h) (5-10 mU) is incubated with 20 mM HEPES pH 7.4, 0.03% Triton X-100, 0.1 mM CaCl₂, 0.1 mg/ml phosphatidylserine, 10 μg/ml diacylglycerol, 50 μM ERMRPRKRQGSVRRRV, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

PKCL (h)

In a final reaction volume of 25 μl, PKCL (h) (5-10 mU) is incubated with 20 mM HEPES pH 7.4, 0.03% Triton X-100, 50 μM ERMRPRKRQGSVRRRV, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

PKCμ (h)

In a final reaction volume of 25 μl, PKCV (h) (5-10 mU) is incubated with 20 mM HEPES pH 7.4, 0.03% Triton X-100, 30 μM KKLNRTLSVA, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

PKCe (h)

In a final reaction volume of 25 μl, PKC6 (h) (5-10 mU) is incubated with 8 mM MOPS pH 7.0, 0.2 mM EDTA, 0.1 mg/ml histone H1, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

PKCM (h)

In a final reaction volume of 25 μl, PKCQ (h) (5-10 mU) is incubated with 8 mM MOPS pH 7.0, 0.2 mM EDTA, 50 μM ERMRPRKRQGSVRRRV, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

PKD2 (h)

In a final reaction volume of 25 μl, PKD2 (h) (5-10 mU) is incubated with 20 mM HEPES pH 7.4, 0.03% Triton X-100, 30 μM KKLNRTLSVA, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

PKG1P (h)

In a final reaction volume of 25 μl, PKG10 (h) (5-10 mU) is incubated with 8 mM MOPS pH 7.0, 0.2 mM EDTA, 10 μM cGMP, 200 μM RRRLSFAEPG, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

Plk3 (h)

In a final reaction volume of 25 μl, Plk3 (h) (5-10 mU) is incubated with 8 mM MOPS pH 7.0, 0.2 mM EDTA, 2 mg/ml casein, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

PRAK (h)

In a final reaction volume of 25 μl, PRAK (h) (5-10 mU) is incubated with 50 mM Na R-glycerophosphate pH 7.5, 0.1 mM EGTA, 30 μM KKLRRTLSVA, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 50 mM phosphoric acid and once in methanol prior to drying and scintillation counting.

PRK2 (h)

In a final reaction volume of 25 μl, PRK2 (h) (5-10 mU) is incubated with 50 mM Tris pH 7.5, 0.1 mM EGTA, 0.1% R-mercaptoethanol, 30 μM AKRRRLSSLRA, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

Pyk2 (h)

In a final reaction volume of 25 μl, Pyk2 (h) (5-10 mU) is incubated with 8 mM MOPS pH 7.0, 0.2 mM EDTA, 0.1 mg/ml poly(Glu, Tyr) 4:1, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted

onto a Filtermat A and washed three times for 5 minutes in 75 mM phosphoric acid and once in methanol prior to drying and scintillation counting.

p70S6K (h)

In a final reaction volume of 25 μl, p70S6K (h) (5-10 mU) is incubated with 8 mM MOPS pH 7.0, 0.2 mM EDTA, 10011M KKRNRTLTV, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

Ret (h)

In a final reaction volume of 25 μl, Ret (h) (5-10 mU) is incubated with 8 mM MOPS pH 7.0, 0.2 mM EDTA, 250 μM KKKSPGEYVNIEFG, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

RIPK2(h)

In a final reaction volume of 25 μl, RIPK2 (h) (5-10 mU) is incubated with 8 mM MOPS pH 7.0, 0.2 mM EDTA, 0.33 mg/ml myelin basic protein, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted

onto a P30 filtermat and washed three times for 5 minutes in 75 mM phosphoric acid and once in methanol prior to drying and scintillation counting.

ROCK-I (h)

In a final reaction volume of 25 μl, ROCK-I (h) (5-10 mU) is incubated with 8 mM MOPS pH 7.0, 0.2 mM EDTA, 30 μM KEAKEKRQEQIAKRRRLSSLRASTSKSGGSQK, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

ROCK-II (h)

In a final reaction volume of 25 μl, ROCK-II (h) (5-10 mU) is incubated with 50 mM Tris pH 7.5, 0.1 mM EGTA,

30 μM KEAKEKRQEQIAKRRRLSSLRASTSKSGGSQK, 10 mM MgAcetate and [y-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

ROCK-II (r)

In a final reaction volume of 25 μl, ROCK-II (r) (5-10 mU) is incubated with 50 mM Tris pH 7.5, 0.1 mM EGTA, 30 μM KEAKEKRQEQIAKRRRLSSLRASTSKSGGSQK, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

Ron (h)

In a final reaction volume of 25 μl, Ron (h) (5-10 mU) is incubated with 8 mM MOPS pH 7.0, 0.2 mM EDTA, 250 μM KKSRGDYMTMQIG, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

Ros (h)

In a final reaction volume of 25 μl, Ros (h) (5-10 mU) is incubated with 8 mM MOPS pH 7.0, 0.2 mM EDTA, 10 mM MnCl₂, 250 μM KKKSPGEYVNIEFG, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

Rse (h)

In a final reaction volume of 25 μl, Rse (h) (5-10 mU) is incubated with 8 mM MOPS pH 7.0, 0.2 mM EDTA, 250 μM KVEKIGEGTYGVVYK, 1 mM MnCl₂, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

Rsk1 (h)

In a final reaction volume of 25 μl, Rsk1 (h) (5-10 mU) is incubated with 8 mM MOPS pH 7.0, 0.2 mM EDTA, 30 μM KKKNRTLSVA, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

Rsk1 (r)

In a final reaction volume of 25 μl, Rsk1 (r) (5-10 mU) is incubated with 8 mM MOPS pH 7.0, 0.2 mM EDTA, 30 μM KKKNRTLSVA, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

Rsk2 (h)

In a final reaction volume of 25 μl, Rsk2 (h) (5-10 mU) is incubated with 8 mM MOPS pH 7.0, 0.2 mM EDTA, 30 μM KKKNRTLSVA, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

Rsk3 (h)

In a final reaction volume of 25 μl, Rsk3 (h) (5-10 mU) is incubated with 8 mM MOPS pH 7.0, 0.2 mM EDTA, 30 μM KKKNRTLSVA, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

SAPK2a (h)

In a final reaction volume of 25 μl, SAPK2a (h) (5-10 mU) is incubated with 25 mM Tris pH 7.5, 0.02 mM EGTA, 0.33 mg/ml myelin basic protein, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

SAPK2b (h)

In a final reaction volume of 25 μl, SAPK2b (h) (5-10 mU) is incubated with 25 mM Tris pH 7.5, 0.02 mM EGTA, 0.33 mg/ml myelin basic protein, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

SAPK3 (h)

In a final reaction volume of 25 μl, SAPK3 (h) (5-10 mU) is incubated with 25 mM Tris pH 7.5, 0.02 mM EGTA, 0.33 mg/ml myelin basic protein, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

SAPK4 (h)

In a final reaction volume of 25 μl, SAPK4 (h) (5-10 mU) is incubated with 25 mM Tris pH 7.5, 0.02 mM EGTA, 0.33 mg/ml myelin basic protein, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

SGK (h)

In a final reaction volume of 25 μl, SGK (h) (5-10 mU) is incubated with 8 mM MOPS pH 7.0, 0.2 mM EDTA, 30 μM GRPRTSSFAEGKK, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

SGK2 (h)

In a final reaction volume of 25 μl, SGK2 (h) (5-10 mU) is incubated with 8 mM MOPS pH 7.0, 0.2 mM EDTA, 30 μM GRPRTSSFAEGKK, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

SGK3 (h)

In a final reaction volume of 25 μl, SGK3 (h) (5-10 mU) is incubated with 8 mM MOPS pH 7.0, 0.2 mM EDTA, 250 μM GRPRTSSFAEGKK, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

Snk (h)

In a final reaction volume of 25 μl, Snk (h) (5-10 mU) is incubated with 8 mM MOPS pH 7.0, 0.2 mM EDTA,

2 mg/ml casein, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

Syk (h)

In a final reaction volume of 25 μl, Syk (h) (5-10 mU) is incubated with 50 mM Tris pH 7.5, 0.1 mM EGTA, 0.1 mM Na3VO4, 0.1% R-mercaptoethanol, 0.1 mg/ml poly(Glu, Tyr) 4:1, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a Filtermat A and washed three times for 5 minutes in 75 mM phosphoric acid and once in methanol prior to drying and scintillation counting.

TAK1 (h)

In a final reaction volume of 25 μl, TAK1 (h) (5-10 mU) is incubated with 8 mM MOPS pH 7.0, 0.2 mM EDTA, 2 mg/ml casein, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

TBK1 (h)

In a final reaction volume of 25 μl, TBK1 (h) (5-10 mU) is incubated with 8 mM MOPS pH 7.0, 0.2 mM EDTA, 200 μM KRRRALS(p)VASLPGL, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

Tie2 (h)

In a final reaction volume of 25 μl, Tie2 (h) (5-10 mU) is incubated with 8 mM MOPS pH 7.0, 0.2 mM EDTA, 0.5 mM MnCl₂, 0.1 mg/ml poly(Glu, Tyr) 4:1, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a Filtermat A and washed three times for 5 minutes in 75 mM phosphoric acid and once in methanol prior to drying and scintillation counting.

TrkA (h)

In a final reaction volume of 25 μl, TrkA (h) (5-10 mU) is incubated with 8 mM MOPS pH 7.0, 0.2 mM EDTA,

250 μM KKKSPGEYVNIEFG, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

TrkB (h)

In a final reaction volume of 25 μl, TrkB (h) (5-10 mU) is incubated with 8 mM MOPS pH 7.0, 0.2 mM EDTA, 0.1 mg/ml poly(Glu, Tyr) 4:1, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a Filtermat A and washed three times for 5 minutes in 75 mM phosphoric acid and once in methanol prior to drying and scintillation counting.

TSSK2 (h)

In a final reaction volume of 25 μl, TSSK2 (h) (5-10 mU) is incubated with 8 mM MOPS pH 7.0, 0.2 mM EDTA, 10011M KKKVSRSGLYRSPSMPENLNRPR, 10 mM MgAcetate and [y-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

Yes (h)

In a final reaction volume of 25 μl, Yes (h) (5-10 mU) is incubated with 8 mM MOPS pH 7.0, 0.2 mM EDTA, 0.1 mg/ml poly(Glu, Tyr) 4:1, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a Filtermat A and washed three times for 5 minutes in 75 mM phosphoric acid and once in methanol prior to drying and scintillation counting.

ZAP-70 (h)

In a final reaction volume of 25 μl, ZAP-70 (h) (5-10 mU) is incubated with 50 mM Tris pH 7.5, 0.1 mM EGTA, 0.1 mM Na3VO4, 0.1% R-mercaptoethanol, 0.1 mg/ml poly(Glu, Tyr) 4:1, 10 mM MnCl₂, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a Filtermat A and washed three times for 5 minutes in 75 mM phosphoric acid and once in methanol prior to drying and scintillation counting.

ZIPK (h)

In a final reaction volume of 25 μl, ZIPK (h) (5-10 mU) is incubated with 8 mM MOPS pH 7.0, 0.2 mM EDTA, 250 μM KKLNRTLSFAEPG, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

### Example 13

### Effects of Compounds on Ribosomal RNA Synthesis

Assays can also be conducted to determine the effects of compounds on rRNA synthesis from 45S rDNA. Synthesized rRNA is quantified by a polymerase chain reaction (PCR) assay. A primer/probe set can be designed using Primer Express software and synthesized by a commercial supplier, such as Applied Biosystems. A 5′ ETS Probe having the following sequence (@ its 3′ end): 6FAM-TTG ATC CTG CCA GTA GC-MGBNFQ is used. The primer sequences are as follows:

| | |
|---|---|
| Forward Primer: | CCG CGC TCT ACC TTA CCT ACC T |
| Reverse Primer: | GCA TGG CTT AAT CTT TGA GAC AAG. |

A control assay that detects effects of the compounds on C-myc transcription can also be conducted using a primer/probe set, that can be purchased from ABI (TaqMan Gene Expression Assay with assay ID: Hs99999003_m1). The following assay protocol is utilized:

Step 1. Reverse transcription of RNA to DNA

Mix
1 ug RNA
2.5 ul 10×Taq Man buffer
5.5 ul 25 mM MgCl2
5 ul of a mix of dNTP (500 uM each)
1.2 ul random hexamer primer (2.5 uM stock)
0.5 ul RNase inhibitor (0.4 units/ul)
0.6 ul Reverse Transcriptase (1.2 units/ul)
bring to 25 ul total volume with water

Incubate at 48 degrees C. for 30 minutes

Inactivate Reverse Transcriptase by incubating at 95 for 5 minutes

Step 2. PCR

Mix

PCR cycles
5 ul Reverse Transcriptase reaction product
12.5 μl 2×PCR mix
1 uM forward primer
1 uM reverse primer
0.5 uM Taq Man probe
500 nM Rox
Adjust to 25 ul final volume with water

PCR Cycles
95 degrees C 1 minute
40 cycles of
95 degrees C 15 seconds
60 degrees C 1 minute.

A representative cell-proliferation assay protocol using Alamar Blue dye (stored at 4° C., use 20 ul per well) is described below. This assay monitors the reducing potential of metabolically active proliferating cells: proliferating cells reduce the Alamar Blue to form a fluorescent product, while non-proliferating cells and dying cells do not. Thus the proliferating cells can be counted using a fluorescence visualization method to compare the effects of the test compounds.

96-Well Plate Setup and Compound Treatment
a. Split and trypsinize cells.
b. Count cells using hemocytometer.
c. Plate 4,000-5,000 cells per well in 100 μl of medium and seed into a 96-well plate according to the following plate layout. Add cell culture medium only to wells B10 to B12. Wells B1 to B9 have cells but no compound added.

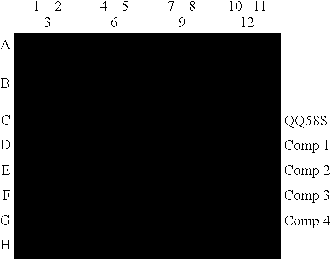

d. Add 100 μl of 2× drug dilution to each well in a concentration shown in the plate layout above. At the same time, add 100 μl of media into the control wells (wells B10 to B12). Total volume is 200 μl/well.
e. Incubate four (4) days at 37° C., 5% CO₂ in a humidified incubator.
f. Add 20 μl Alamar Blue reagent to each well.
g. Incubate for four (4) hours at 37° C., 5% CO₂ in a humidified incubator.
h. Record fluorescence at an excitation wavelength of 544 nm and emission wavelength of 590 nm using a microplate reader.

In the assays, cells are cultured with a test compound for approximately four days, the dye is then added to the cells, and fluorescence of non-reduced dye is detected after approximately four hours. Different types of cells can be utilized in the assays. Pancreatic cancer cells (i.e., MiaPaca), colorectal cancer cells (i.e., HCT-116) and cervical cancer cells (i.e., HeLa) can be treated in the assays with compounds such as those described herein, and their effects on cell proliferation can be observed. Using this assay, the compounds of the invention can be evaluated for general cytotoxicity, as well as tissue-specific cytotoxicity and cancer cell-specific cytotoxicity.

Using the methods described above, the following compounds were shown to have activity in the HCT-116 assay:

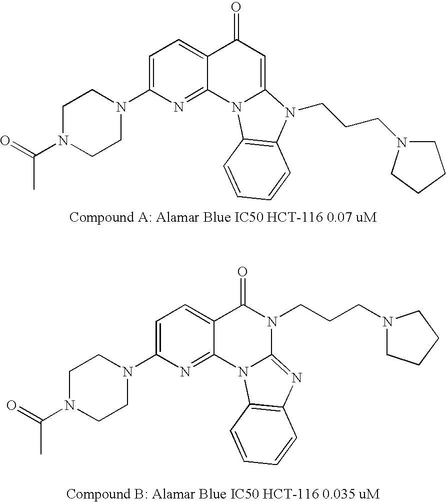

### Example 14

### Methodology for In Vivo Assessment

6 wk old female nu/nu mice can be purchased from Simonsen Labs, Gilroy, Calif. They would then be injected with 5×10⁶HCT116 cells SQ in right flank. When tumors reach sufficient size for study, they would be randomized into groups. Tumor sizes would be evaluated by standard methods for determining the volume of the tumors in animals prior to and after treatment.

Animals would be dosed by bolus injection —IV through lateral tail vein for fourteen consecutive days. Caliper measurements would be taken on Day 1, 3, 5, 7, 10, 12, 14, and 18.

### Example 15

### Cell Proliferation and/or Cytotoxicity Assay

The antiproliferative effects of the present compounds may be tested using a cell proliferation and/or cytotoxicity assay, following protocols described below.

Cell culture. Human cervical epithelial cells (HeLa cells) are obtained from American Type Culture Collection (Manassas, Va.). Cells are grown in Eagle's minimum essential medium (MEM, Hyclone, Utah) supplemented with 2 mM Glutamine, 0.1 mM nonessential amino acid, 1 mM Na Pyruvate, 1.5 g/L NaHCO₃, 50 mg/L gentamicin, and 10% fetal bovine serum (Hyclone, USA) in a humidified atmosphere of 5% CO₂ at 37° C.

MTS assays. Antiproliferative effects of anticancer drugs are tested by the CellTiter 96 AQᵤₑₒᵤₛ assay (Promega, WI), which is a colorimetric assay for determining the number of viable cells. (See, e.g., Wang, L., et al., Methods Cell Sci (1996) 18:249-255). Generally, cells (2,000 to 5,000 cells/well) are seeded on 96 well flat bottom plates (Corning, N.Y.) in 100 μl of culture medium without any anticancer drug on day 0, and the culture medium is exchanged for that contained anticancer drugs at various concentrations on day 1. After incubation for 3 days under normal growth conditions (on day 4), the monolayers are washed once in PBS, and the medium is switched to 100 μl of PBS in each of the 96 well plate. After mixing MTS and PMS at the ratio of 20:1, 20 μl of MTS/PMS solution is added to each of the 96 well plate and incubated for 4 hours in a humidified atmosphere of 5% CO₂ at 37° C. The absorbance is read at 490 nm using FLUOstar Galaxy 96 well plate reader (BMG Labtechnologies, Germany).

### Example 16

### Measurement of mRNA values in Cell Assays

Real-time quantitative PCR (QPCR) method may be used to detect the changes of the target c-myc and the endogenous reference GAPDH gene copies in the same tube. Generally, cells (15,000 cells/well) are seeded on 96 well flat bottom plates (Corning, N.Y.) and incubated under normal growth conditions for overnight. The next day, the culture medium is exchanged for that containing anticancer drugs at various concentrations and incubated for 4 hrs in a humidified atmosphere of 5% CO₂ at 37° C. Total RNA (tRNA) is extracted using the RNeasy 96 Kit (QIAGEN, CA). The concentration of the tRNA is determined by the RiboGreen RNA Quantitation Reagent (Molecular Probes, OR).

A reverse-transcription (RT) reaction may be conducted using 50 ng of tRNA from each well in a 25 μl reaction containing 1×TaqMan RT buffer, 2.5 uM random hexamers, 5.5 mM MgCl₂, 0.5 mM each deoxynucleoside triphosphate (dNTP), 30 U MultiScribe Reverse Transcriptase, and 10 U RNase inhibitor. RT reactions are incubated for 10 min at 25° C., reverse-transcribed for 30 min at 48° C., inactivated for 5 min at 95° C., and placed at 4° C. All RT reagents may be purchased from Applied Biosystems, CA.

Real-Time QPCR reaction may be performed in a 50 μl reaction containing the 5 μl of cDNA, 1× Universal PCR Master Mix, 1× c-myc Pre-Developed Primers and Probe set, and 0.8×GAPDH Pre-Developed Primers and Probe set. Because of the relative abundance of GAPDH gene in Hela, GAPDH primers and probe concentration may be adjusted to get accurate threshold cycles (C_{T}) for both genes in the same tube. The threshold cycle (C_{T}) indicates the fractional cycle number at which the amount of amplified target reaches a fixed threshold. By doing so, the GAPDH amplification is stopped before it can limit the common reactants available for amplification of the c-myc. The ΔRn value represents the normalized reporter signal minus the baseline signal. ΔRn increases during PCR as amplicon copy number increases until the reaction approaches a plateau.

The c-myc probe is labeled with 6FAM™ dye-MGB and the GAPDH probe is labeled with VIC™ dye-MGB. Preincubation is performed for 2 min at 50° C. to activate AmpErase UNG enzyme and then for 10 min at 95° C. to activate AmpliTaq DNA Polymerase. DNA is amplified for 40 cycles of 15 sec at 95° C. and 1 min at 60° C. Human c-myc and GAPDH cDNA are amplified, detected, and quantitated in real time using the ABI Prism 7000 Sequence Detection system (Applied Biosystems, CA), which is set to detect both 6-FAM and VIC reporter dyes simultaneously.

The data may be analyzed using the ABI PRISM Sequence Detection System and Microsoft Excel. Relative quantitation is done using the standard curve and comparative C_{T} method at the same time, and both methods gave equivalent results. The cycle at which the amplification plot crosses the C_{T} is known to accurately reflect relative mRNA values. (See, Heid, et al., Genome Res. (1996) 6:986-994; Gibson, et al., Genome Res. (1996) 6:995-1001). QPCR reactions are set up in triplicate at each cDNA sample and the triplicate C_{T} values are averaged. All reagents including Pre-Developed Primers and probe set may be purchased from Applied Biosystems, CA.

### Example 17

### In Vitro Characterization

Various methods may be used for certain in vitro characterization of the compounds of the present invention, including but not limited to i) stop assays; ii) quadruplex/duplex competition assay; iii) quadrome footprints; and iv) direct assay in the absence of a competitor molecule.

Stop Assays. Stop assays are high throughput, first-pass screens for detecting drugs that bind to and stabilize the target G-quadruplex. Generally, DNA template oligonucleotide is created, which contains the nucleotide sequence of the “target” quadruplex against which drug screening is desired. A fluorescently labeled primer DNA is then annealed to the 3′ end of the template DNA. A DNA polymerase such as Taq polymerase is then introduced to synthesize a complementary strand of DNA by extending from the fluorescently labeled primer. When the progress of the Taq polymerase is unhindered, it synthesizes a full-length copy of the template. Addition of a test drug that merely binds to duplex DNA but does not bind selectively the quadruplex region results in a decrease in synthesis of full length product and a concomitant increase in variable-length DNA copies. If, however, the test drug selectively binds to and stabilizes the quadruplex, the progress of polymerase arrests only at the quadruplex, and a characteristic “Stop Product” is synthesized.

Compounds are initially screened at a single concentration, and “hits” are re-assayed over a range of doses to determine an IC₅₀ value (i.e., the concentration of drug required to produce an arrest product/full-length product ratio of 1:1). These products are visualized by capillary electrophoresis.

Quadruplex/Duplex Competitor Assay. The selectivity of compounds for the target quadruplex sequence relative to duplex DNA may be measured using a competition assay (i.e., “selectivity screen”). This selectivity screen uses the stop assay as a reporter system to measure the relative ability of an externally added DNA sequence to compete with the target quadruplex structure formed in the DNA template for binding of the drug. For example, the competitors are the c-myc quadruplex sequence, which is identical to the quadruplex sequence present in the template DNA; or a plasmid DNA which mimics complex genomic duplex DNA. The degree to which each competitor successfully “soaks up” drug in solution is reflected by the quantitative decrease in synthesis of the stop product. In this manner, the relative binding affinities of drug to both the target quadruplex and duplex DNA are determined.

Quadrome Footprints. Compounds may also be evaluated for their ability to bind to other native quadruplex structures of biological relevance, including quadruplex control elements that regulate a range of different oncogenes. The resulting data are used to create a Quadrome footprint.

Direct Interaction Assay. Compounds may be evaluated for their ability to interact directly with nucleic acids capable of forming a quadruplex structure, wherein the nucleic acid is not a telomeric nucleic acid. The assay may be performed in the same or different vessels. For example, a compound may be contacted with each nucleic acid in the same vessel. Alternatively, a compound may be separately contacted with each of the nucleic acids tested in a different vessel. A telomeric nucleic acid as used herein represents a region of highly repetitive nucleic acid at the end of a chromosome. As used herein, a direct interaction is measured without the presence of a competitor nucleic acid.

An interaction between the compound and the nucleic acid may be determined for example, by measuring IC₅₀ values, which are indicative of the binding and/or quadruplex stabilization. The selectivity of interactions may be determined, for example, by comparing measured IC₅₀ values. For example, the lowest IC₅₀ values may be used to indicate a strong interaction between the compound and the nucleic acid, while highest IC₅₀ values show a poor interaction; thus, showing selectivity of interaction. The reaction products may be characterized by capillary electrophoresis.

### Example 18

### Direct Interaction Assay

Generally, a 5′-fluorescent-labeled (FAM) primer (P45, 15 nM) is mixed with template DNA (15 nM) in a Tris-HCL buffer (15 mM Tris, pH 7.5) containing 10 mM MgCl₂, 0.1 mM EDTA and 0.1 mM mixed deoxynucleotide triphosphates (dNTP's). The mixture is denatured at 95° C. for 5 minutes and, after cooling down to room temperature, is incubated at 37° C. for 15 minutes. After cooling down to room temperature, 1 mM KCl₂ and the test compound (various concentrations) are added and the mixture incubated for 15 minutes at room temperature.

The primer extension is performed by adding 13 mM KCl and Taq DNA Polymerase (2.5 U/reaction, Promega) and incubating at 70° C. for 20 minutes. The reaction is stopped by adding 1 μl of the reaction mixture to 10 μl Hi-Di Formamide mixed and 0.25 μl LIZ120 size standard. The method is repeated with the addition of various concentrations of competitor nucleic acids at the first step, along with the primer and template sequences. The G-quadruplex binding ligand is added at the concentration previously established to produce a 1:1 ratio of stop-product to full-length product. A CC50 for each nucleic acid competitor is defined as the concentration of competitor required to change the ratio of arrest product to full-length product from 1:1 to 1:2. The nucleic acid sequences of quadruplexes that may be used for this assay are set forth in Table 4.

TABLE 4
(STOP TEMPLATES)

TGFB3-81
TATACGGGGTGGGGGAGGGAGGGATTAGCGACACGCAATTGCTATAGTGA
GTCGTATTAGCTACGTACAGTCAGTCAGACT
HRAS-85
TATACCGGGGCGGGGCGGGGGCGGGGGCTTAGCGACACGCAATTGCTA
TAGTGAGTCGTATTAGCTACGTACAGTCAGTCAGACT
BCL2-97(full)
TAGGGGCGGGCGCGGGAGGAAGGGGGCGGGAGCGGGGCTGTTAGCGA
CACGCAATTGCTATAGTGAGTCGTATTAGCTACGTACAGTCAGTCAGACT
HMGA-97
TTAGAGAAGAGGGGAGGAGGAGGAGGAGAGGAGGAGGCGCTTAGCGAC
ACGCAATTGCTATAGTGAGTCGTATTAGCTACGTACAGTCAGTCAGACT
MYC99
TCCAACTATGTATACTGGGGAGGGTGGGGAGGGTGGGGAAGGTTAGCGA
CACGCAATTGCTATAGTGAGTCGTATTAGCTACGTACAGTCAGTCAGACT
IMOTIF99
TCCAACTATGTATACCCTTCCCCACCCTCCCCACCCTCCCCATTAGCGAC
ACGCAATTGCTATAGTGAGTCGTATTAGCTACGTACAGTCAGTCAGACT
Humtel-95
TCATATATGACTACTTAGGGTTAGGGTTAGGGTTAGGGTTACTGCCACGC
AATTGCTATAGTGAGTCGTATTAGCTACGTACAGTCAGTCAGACT
SRC89
ATGATCACCGGGAGGAGGAGGAAGGAGGAAGCGCGCTGCCACGCAATT
GCTATAGTGAGTCGTATTAGCTACGTACAGTCAGTCAGACT
Primer:
(45 MER)
AGTCTGACTGACTGTACGTAGCTAATACGACTCACTATAGCAATT

### Example 19

### Cytochrome P450 (CYP450) Inhibition Assay

The compounds of the present invention may be evaluated for potential inhibitory activity against cytochrome P450 isoenzymes. Generally, six reaction tubes with 100 μL of a solution containing 50 mM potassium phosphate, pH 7.4, 2.6 mM NADP+, 6.6 mM glucose 6-phosphate, 0.8 U of glucose 6-phosphate dehydrogenase/mL and 1:6 serial dilutions of the test compound will be prepared along with six tubes of 1:6 serial dilutions of a suitable positive control inhibitor. The reactions will be initiated by adding 100 μL of a pre-warmed enzyme/substrate solution to the reaction tubes. A zero time-point control reaction will be prepared by adding 50 μL of acetonitrile to 100 μL of cofactor solution to inactivate the enzymes, then adding 100 μL of enzyme/substrate solution. A control reaction with no inhibitor may also be prepared. After a suitable incubation at 37 C, the reactions will be terminated by the addition of 50 μL of acetonitrile. The reactions will be analyzed for the metabolite forms of the probe substrate using LC/MS/MS.

### Example 20

### Evaluation of Compound Efficacy in Tumor Suppression

A representative experiment for evaluating the efficacy of compounds of the present invention in athymic nude mouse models of human carcinoma may be designed as follows. Male or female animals (mouse, Sim) (NCR, nu/nu) aged five to six weeks and weighing more than 20 grams will be used. The animals will be purposely bred and will be experimentally naïve at the outset of the study. Tumors will be propagated either from injected cells or from the passage of tumor fragments. Cell lines to be used include, but are not limited to, alia Paca-2, HPAC, Hs700T, Panc10.05, Panc 02.13, PL45, SW 190, Hs 766T, CFPAC-1 and PANC-1.

Cell implantation. One to ten million cells suspended in 0.1 ml culture media with or without Matrigel (Collaborative Biomedical Products, Inc, Bedford, Mass.) will be inoculated subcutaneously in the right flank of sixty animals. There will only be one injection per animal. Within 7-14 days of injection tumors will develop to a study use size of approximately 1.0 cm³. A small subset (<10/60) animals will be considered. Donors and tumors will be grown 10-28 days and to a size of 1.5 cm³ in order to be used for serial transplantation.

Fragment transplantation. Donor animals will be euthanized and tumors surgically excised and cut into 2 mm³ size fragments using aseptic technique. Animals to be implanted will be lightly anesthetized with isoflurane. The area to implanted will be cleansed with 70% alcohol and betadine. A single fragment will then be implanted subcutaneously using a trocar.

Efficacy studies. Groups of 50-60 tumor bearing animals will be randomly divided. For example, in a representative study, animals may be randomly divided into three to eight groups containing 7 animals each, as described in Table 5.

TABLE 5

| | Number | | | Dose | Number |
|---|---|---|---|---|---|
| | of | | | Solution | Euthanized |
| Group | Males/ | | Dose Vol. | Conc. | on: |
| No. | Females | Dose Level | (μL) | (mg/mL) | Day 28-42 |
| 1 | N = 7 | Negative | 250 | | all |
| | | Control* | | | |
| 2 | N = 7 | Positive | 10-400 | 2 to 5 IP | all |
| | | Control** | IP | 2.5 to 5 IV | |
| | | | 10-250 | ≦10 PO | |
| | | | IV | | |
| | | | 125-500 | | |
| | | | PO | | |
| Groups | N = 7/ | Test | 10-400 | 2.5 to 5 IP | all |
| 3-8 | grp | Compound | IP | 2.5 to 5 IV | |
| | <56 | 1 to 25 IP | 10-250 | 10 PO | |
| | total | 1 to 50 IV | IV | | |
| | | 50 to 200 PO | 125-500 | | |
| | | | PO | | |

*Vehicle/Diluent
**Commercially available anticancer compounds including, but not limited to, Taxol, CPT11 and Gemcitabine will be used as positive controls.

Dosing Procedure. Compounds will be administered QD, QOD, Q3D or once weekly via IP, IV (lateral tail vein) or PO. Animals will be dosed in a systematic order that distributes the time of dosing similarly across all groups. For bolus IP and PO dosing, animals will be manually restrained. For IV bolus dosing or short term IV infusion (one minute), animals will be mechanically restrained but not sedated. Disposable sterile syringes will be used for each animal/dose. A test compound in combination with about 10-100 mg/kg (e.g., about 40 mg/kg) chemotherapeutic agent such as gemcitabine also will be tested, normally by IP administration once per week.

### Example 21

### Evaluation of Maximum Tolerated Doses

A representative experiment for evaluating the maximum tolerate dose (MTD) of compounds of the present invention may be designed as follows. Selection for animal models is as described in Example 101.

Acute Toxicity Studies. In a representative study to determine the MTD after a single dose, sixty naive animals, for example, will be randomly divided into groups containing 10 animals (5 male and 5 female) and will receive either one compound via two routes of administration or two compounds via a single route of administration. A single 50 mg/kg IV dose has been shown to be tolerated, and is used as the preliminary low dose levels. The low dose for oral studies is based on projected tolerability and will be adjusted downward if necessary. A representative design of dose levels, dose volumes and dose solution concentration are described in Table 6.

TABLE 6

| | Number | | | | |
|---|---|---|---|---|---|
| | of | | | Dose | Number |
| | Males | | | Solution | Euthanized |
| Group | and | Dose Level | Dose Vol. | Conc. | on: |
| No. | Females | (mg/kg) | (μL) | (mg/mL) | Day 7 |
| 1 | N = 5 M | Test | 250 IV | 5 IV | all |
| | N = 5 F | compound | 500 PO | 5 PO | |
| | | #1 | | | |
| | | 50 IV | | | |
| | | 100 PO | | | |
| 2 | N = 5 M | Test | 250 IV | 8.25 IV | all |
| | N = 5 F | compound | 500 PO | 10 PO | |
| | | #1 | | | |
| | | 75 IV | | | |
| | | 200 PO | | | |
| 3 | N = 5 M | Test | 250 IV | 10 IV | all |
| | N = 5 F | compound | 500 PO | 15 PO | |
| | | #1 | | | |
| | | 100 IV | | | |
| | | 300 PO | | | |
| 4 | N = 5 M | Test | 250 IV | 5 IV | all |
| | N = 5 F | compound | 500 PO | 5 PO | |
| | | #2 | | | |
| | | 50 IV | | | |
| | | 100 PO | | | |
| 5 | N = 5 M | Test | 250 IV | 8.25 IV | all |
| | N = 5 F | compound | 500 PO | 10 PO | |
| | | #2 | | | |
| | | 75 IV | | | |
| | | 200 PO | | | |
| 6 | N = 5 M | Test | 250 IV | 10 IV | all |
| | N = 5 F | compound | 500 PO | 15 PO | |
| | | #2 | | | |
| | | 100 IV | | | |
| | | 300 PO | | | |

SubChronic Studies. In a representative study to characterize dose-response relationships following repeated dosing, twenty-five naive animals, for example, will be randomly divided into groups containing 5 animals each as described in Table 7. Each two week study will test only one compound via a single route of administration at an optimal dose derived from data collected in prior acute toxicity studies.

TABLE 7

| | Number | | | | |
|---|---|---|---|---|---|
| | of | | | Dose | Number |
| | Males | | | Solution | Euthanized |
| Group | or | Dose Level | Dose Vol. | Conc. | on: |
| No. | Females | (mg/kg) | (μL) | (mg/mL) | Day 14 |
| 1 | N = 5 | Negative | 250 IV | Depends | all |
| | | Control | 500 PO | on Dose | |
| | | | | Level | |
| 2 | N = 5 | Test | 250 IV | Depends | all |
| QD | | Compound As | 500 PO | on Dose | |
| | | Determined in | | Level | |
| | | MTD Studies | | | |
| 3 | N = 5 | Test | 250 IV | Depends | all |
| QOD | | Compound As | 500 PO | on Dose | |
| | | Determined in | | Level | |
| | | MTD Studies | | | |
| 4 | N = 5 | Test | 250 IV | Depends | all |
| Q3D | | Compound As | 500 PO | on Dose | |
| | | Determined in | | Level | |
| | | MTD Studies | | | |
| 5 | N = 5 | Test | 250 IV | Depends | all |
| Q7D | | Compound As | 500 PO | on Dose | |
| | | Determined in | | Level | |
| | | MTD Studies | | | |

Dosing Procedure. Compounds will be administered QD, QOD, Q3D or Q7D via IV (lateral tail vein) or PO. Animals will be dosed in a systematic order that distributes the time of dosing similarly across all groups. For PO dosing, animals will be manually restrained. For IV bolus dosing or short term IV infusion (one minute), animals will be mechanically restrained but not sedated. Disposable sterile syringes will be used for each animal/dose.

### Example 22

### Evaluation of Pharmacokinetic Properties

A representative pharmacokinetic study for evaluating pharmacokinetic properties of the compounds herein may be designed as follows. Male animals (mouse, Balb/c or rat, SD) aged five to six weeks. For rat models, rats weighing more than 200 grams will be used. In a representative study, twenty animals, for example, will randomly divided into 4 groups, as shown in Table 8. One group with be untreated and samples taken to be used as a base line. The other three groups will be and administered a single dose of compounds by intravenous injection.

TABLE 8

| | No. | |
|---|---|---|
| Group | of | Time followed by |
| No. | Animals | injection (h) |
| 1 | 2 | Naïve |
| 2 | 6 | .25, 2, 8 |
| 3 | 6 | .5, 4, 12 |
| 4 | 6 | 1, 6, 24 |

Dosing Procedure. Compounds will be administered via IV (lateral tail vein), IP or PO. Animals will be dosed in a systematic order that distributes the time of dosing similarly across all groups. For IP and PO dosing, animals will be manually restrained. For IV bolus dosing or short term IV infusion (one minute), animals will be mechanically restrained but not sedated. Disposable sterile syringes will be used for each animal/dose.

Approximately 0.5 ml of blood will be collected from the naive animals via cardiac puncture prior to the first dose Terminal blood samples (0.5 ml) will be collected via cardiac puncture from two animals per group per time point according to the above chart. All samples will be placed in tubes containing lithium heparin as anticoagulant and mixed immediately by inverting. They will be centrifuged and the plasma flash frozen in liquid nitrogen, stored at ±70° C. or greater and analyzed for drug levels.

### Example 23

### Determination of In Vitro Metabolic Stability in Hepatocytes

A representative protocol to determine the stability of a new chemical entity in the presence of hepatocytes (human, rat, dog, monkey) in in vitro incubations may be designed as follows. The test article will be incubated with hepatocytes and suitable media for various times at 37° C. The reaction mixtures will be extracted and analyzed by LC/MS/MS for the parent compound and anticipated metabolites. If applicable, a half-life will be calculated for the consumption of the test article. Metabolism controls will be run for comparison of the half-life values with that obtained for the test article. The metabolism controls may be tolbutamide, desipramine and naloxone, which have defined pharmacokinetics corresponding to low, moderate and high in vivo clearance values, respectively.

Metabolic Stability Study. Generally, solutions of the test compounds will be prepared along with a cocktail solution of metabolism controls that are intended to provide a reference for enzyme activity. The reactions will be initiated by combining these pre-warmed solutions with hepatocyte suspensions and with a media control solution. Control zero samples will be taken from these reactions immediately after initiation. Additional samples may be taken at appropriate time points. Each sample will be immediately placed in a terminating solution (acidified MeCN containing IS) to stop the reaction. Hepatocyte blank suspensions and test compound standard solutions will be prepared.

Samples and standards for the test compound as well as appropriate blanks may be subjected to a custom sample preparation procedure and analyzed for the parent and/or metabolite form of the test compound using HPLC coupled with tandem mass spectrometry. Samples and standards for the metabolism controls may be subjected to the analytical method described herein. Where Krebs Henseleit buffer will be added, the buffer is bubbled with 5% CO₂ in air at room temperature for 5-10 minutes before adding BSA to a final concentration of 0.2% w/v. The volume of terminating solution and the method of sample preparation will be determined for the test article during method development.

Test Article/Media Solution. A solution of the test article will be prepared by adding an appropriate volume of the stock solution to 0.2% BSA in Krebs Henseleit buffer equilibrated with 5% CO₂ in air. The final concentration will be between 5 μM and 20 μM, and the final assay concentration at initiation of the reactions will be between 1 μM and 10 μM.

Metabolism Controls/Media Solution. A solution of tolbutamide, desipramine and naloxone will be prepared by adding an appropriate volume of each 10 mM stock solution to 0.2% BSA in Krebs Henseleit buffer equilibrated with 5% CO₂ in air. The final concentration will be 20 μM for each metabolism control and the final assay concentration will be 10 μM at initiation of the reactions.

Hepatocyte Suspension Solution. The hepatocytes will be thawed and isolated according to the vendor (Invitrotech, Inc.) instructions. During the final step of the procedure, the viability of the cells will be determined using the method of trypan blue exclusion. Then, the hepatocytes will be resuspended with 0.2% BSA in Krebs Henseleit buffer equilibrated with 5% CO₂ in air so the final concentration is 0.5 million viable cells/mL. The concentration at the initiation of the reactions will be 0.25 million viable cells/mL.

Initiating Test Article Incubation. Equal volumes of the test article solution prepared in step 2.1.3 will be dispensed into four polypropylene scintillation vials. The vials are pre-warmed for 5-10 minutes at 37° C. with 95% humidity and 5% CO₂. Equal volumes of 0.2% BSA in Krebs Henseleit buffer equilibrated with 5% CO₂ in air will be added to two of the vials and mixed thoroughly. Immediately after initiating the reaction, a timer is started and a 100 μL sample is removed from each vial and placed into a 1.7-mL centrifuge tube containing a suitable volume of terminating solution. These samples will serve as media controls to check for non-enzymatic degradation and non-specific binding to the vessel.

Equal volumes of the hepatocyte suspension prepared above will be added to two of the vials and mixed thoroughly. Immediately after initiating the reaction, a timer is started and a 100 μL, sample is removed from each vial and placed into a 1.7-mL centrifuge tube containing a suitable volume of terminating solution. All vials are placed in an incubator maintained at 37° C., 95% humidity and 5% CO₂.

Initiating Metabolism Control Incubation. Equal volumes of the metabolism control solution prepared above will be dispensed into two polypropylene scintillation vials. The vials are pre-warmed for 5-10 minutes at 37° C. with 95% humidity and 5% CO₂. Equal volumes of the hepatocyte suspension prepared above will be added to each of the two vials and mixed thoroughly. Immediately after initiating the reaction, a timer is started and a 100 μL sample is removed from each vial and placed into a 1.7-mL centrifuge tube containing an equal volume of terminating solution. All vials are placed in an incubator maintained at 37° C., 95% humidity and 5% CO₂.

Sample Collection. The vials will be gently shaken and samples (100 μL) will be removed and placed into a 1.7-mL centrifuge tube containing an appropriate volume of terminating solution according to the following schedule: Test article samples are taken after 5, 10, 15, 30, 60, 90 and 120 minutes; metabolism control samples are taken after 30, 60, 90 and 120 minutes. Immediately after removal of the samples, the vials are placed back in the incubator until the last sample is collected.

Blank Preparation. A sample (100 μL) of the hepatocyte suspension will be added to an equal volume of 0.2% BSA in Krebs Henseleit buffer and mixed thoroughly. A 100 μL sample of this solution will be removed and placed into a 1.7-mL centrifuge tube containing the same volume of terminating solution used for the test article reaction. A sample of the incubation medium (0.2% BSA in Krebs Henseleit buffer) will be placed into a 1.7-mL centrifuge tube containing the same volume of terminating solution used for the test article reaction.

Sample Preparation and Analysis. All vials will be centrifuged at 16,000 g for 3 minutes. The supernatants will be placed into polypropylene autosampler vials and stored at 4° C. (<1 day) or −70° C. (>1 day) until analysis. The test article solutions will be analyzed using HPLC/MS/MS conditions according to standard procedures. In one example, the following

HPLC conditions may be used: column (Phenomenex Synergi Hydro-RP, 100.0×2.0 mm, 5 μm); guard column (Phenomenex C18, 4.0×2.0 mm, 5 μm); flow rate (0.3 mL/min); column temperature at 45° C.; injection volume at 10 μL; and ambient autosampler temperature.

### Example 24

### Determination of In Vitro Metabolic Stability in Microsomes

A representative protocol to determine the stability of a new chemical entity in the presence of liver microsomes (human, rat, dog, monkey) in in vitro incubations may be designed as follows. The test article will be incubated with microsomes and suitable media for various times at 37° C. The reaction mixtures will be extracted and analyzed by LC/MS/MS for the parent compound and anticipated metabolites. If applicable, a half-life will be calculated for the consumption of the test article. Metabolism controls will be run for comparison of the half-life values with that obtained for the test article. The metabolism controls are tolbutamide, desipramine and testosterone, and these compounds have defined pharmacokinetics corresponding to low, moderate and high in vivo clearance values, respectively.

Metabolic Stability Study. Generally, six pre-warmed reaction vials with 100 μL of a solution containing 50 mM potassium phosphate, pH 7.4, 2.6 mM NADP⁺, 6.6 mM glucose 6-phosphate, 0.8 U/mL of glucose 6-phosphate dehydrogenase and 1, 10 or 50 μM of the test compound are prepared. Similar reactions with metabolic controls representing low (tolbutamide), moderate (desipramine), and high (testosterone) clearance compounds are run simultaneously with the same enzyme solution. The reactions are initiated by adding 100 μL of a pre-warmed enzyme solution and incubated at 37° C. The zero time-point reaction is prepared by adding 50 μL of acetonitrile (containing internal standard) to the test compound/cofactor solution prior to adding the enzyme solution. After 15, 30, 60, 90 and 120 minutes, a reaction tube is removed from the water bath and the reaction is terminated with 50 μL of acetonitrile containing internal standard. The reactions are extracted and the samples are analyzed for the parent form of the test compound and one metabolite using a C18 column with MS/MS detection. Each assay is performed in duplicate.

Cofactor/Test compound Solution Concentrations. A stock solution of 10 mM NCE will be prepared in 10% DMSO (v/v). For all assays, a 2, 20 or 100 μM solution of the test article will be prepared in 50 mM potassium phosphate, pH 7.4, 2.6 mM NADP⁺, 6.6 mM glucose 6-phosphate and 0.8 U/mL of glucose 6-phosphate dehydrogenase (cofactor solution).

Cofactor/Metabolism Control Solution Concentrations. Stock solutions of the metabolism controls (tolbutamide, desipramine, and testosterone) will be used to prepare a 6 μM solution of the metabolism control in cofactor solution described in step

Enzyme Solution Concentrations. The enzyme solutions will be prepared by adding liver microsomes to 50 mM potassium phosphate, pH 7.4, to a final concentration of 1 mg/mL. All microsomes were purchased from XenoTech or InvitroTech, Inc.

Initiating the Reactions. All the reaction tubes will be pre-warmed at 37° C. in a water bath for about 3-5 minutes. The zero time-point control reaction will be prepared for each replicate by adding 50 μL of acetonitrile containing 15.9 μM nebularine (internal standard) to 100 μL of cofactor solution to inactivate the enzymes, and then vortex mixing. The reactions will be initiated by adding 100 μL of the enzyme solution to each of the tubes and vortex mixing. All the tubes, including the zero time-point control, will be incubated in a 37° C. water bath. The final concentrations of all components in the tubes after initiating the reactions are 50 mM potassium phosphate, pH 7.4, 1.3 mM NADP⁺, 3.3 mM glucose 6-phosphate, 0.4 U/mL of glucose 6-phosphate dehydrogenase, 0.5 mg/mL liver microsomes and 1, 10 or 50 μM test article.

Terminating and Extracting the Reactions. After 15, 30, 60, 90 and 120 minutes at 37° C., the reactions will be terminated by the addition of 150 μL of acetonitrile containing 15.9 μM nebularine (internal standard). The zero time-point control is removed from the water bath after 120 minutes. All vials will be centrifuged at 16,000 g for 3 minutes. The supernatants will be placed into polypropylene autosampler vials and stored at 4° C. (<1 day) or −70° C. (>1 day) until analysis.

Analysis of Test Article Solutions. The test article solutions will be analyzed using HPLC/MS/MS conditions according to standard procedures, such as those described in Example 39.

### Example 25

### Bacterial Mutagenicity Test

This Mutagenicity Assessment assay (Ames Assay) will evaluate the potential of the test article extracts to induce histidine (his) reversion in S. typhimurium (his− to his+) or tryptophan (trp) reversion in E. coli (trp− to trp+) caused by base changes or frameshift mutations in the genome of tester organisms. Generally, a plate incorporation assay will be conducted with five strains of Salmonella typhimurium (TA97a, TA98, TA100, TA102, and TA1535) and one strain of Escherichia coli (WP2-uvrA⁻) in the presence and absence of an exogenous mammalian activation system (S9). The test article will be dissolved in 5% dextrose. A series of dilutions will then be prepared in saline just prior to testing. A Range Finding Study will also be conducted for this assay to determine the appropriate doses for definitive mutagenicity assessment.

Test Material Preparation

A stock solution of test article will be prepared at 20.0 mg/mL as follows: 1.0 g test article will be added to 15.0 mL of 0.1HCl for 1 minute. The test article will be stirred for 15 minutes at room temperature. Next 33.0 mL of deionized water will be added and allowed to stir for 30 minutes. The pH will then be adjusted to 3.53. Lower doses will be prepared by dilution in 5% dextrose from this stock immediately prior to use. To minimize any change of degradation, the test article solutions will be kept on ice after preparation and until just prior to dosing procedures. The test article will be administered in vitro, through a solvent compatible with the test system.

Genotypic Characterization of the Test Strains

Working stocks of test strains will be confirmed for genotypic markers and acceptable spontaneous reversion rates. All working stocks should demonstrate a requirement for histidine or tryptophan (E. coli only). Additionally, the following conformations will be made with each assay, as appropriate: sensitivity to crystal violet due to the rfa wall mutation; sensitivity to ultraviolet light due to the deletion of the uvrB gene (uvrA in E. coli), resistance to ampicillin due to the presence of the pKM101 plasmid; and resistance to tetracycline due to the presence of the pAQ1 plasmid. Spontaneous reversion rates for the strains will be determined using the negative controls.

Test articles that are water-soluble will be dissolved in isotonic saline or other suitable solvent. Test articles that are not water-soluble will be dissolved in dimethylsulfoxide (DMSO) or other suitable solvent. If DMSO is anticipated to cause adverse reactions with the test article, the test article will be suspended in carboxymethylcellulose. In order to aid in dissolution, heating, vigorous vortexing or alternative solvents may be employed.

Test System

This assay will be conducted in accordance with the plate incorporation methodology originally described by Ames (Ames et al., Mutation Research (1975) 31:347-364) and updated by Maron and Ames (Maron et al., Mutation Research (1983) 113:173-215). This assay has historically been used to detect mutation in a gene of a histidine requiring strain to produce a histidine independent strain or concordantly, to detect mutation in a gene of a tryptophan requiring strain to produce a tryptophan independent strain. In addition, it has been shown to detect diverse classes of chemical mutagens which produce heritable DNA mutations of a type which are associated with adverse effects.

The Salmonella typhimurium strains that may be used in this assay, TA97a, TA98, TA100, and TA102 are described by Maron and Ames, supra; Green et al., Mutation Research (1976) 38:33-42); and Brusick et al., Mutation Research (1980) 76:169-190)). S. typhimurium strain TA1535 and E. coli strain Wp2-uvrA⁻ may be obtained from American Type Culture Collection, Manassas, Va. (ATCC numbers: 29629 and 49979, respectively). All working stocks of test strains will be confirmed for genotypic markers and acceptable reversion rates. Working stocks should demonstrate a requirement for histidine or tryptophan (E. coli only).

Experimental Methods

Master plates of the tester strains will be prepared from frozen working stocks. To create working cultures for each bacterial strain used in the assay, a single colony will be transferred from the master plate into Oxoid nutrient broth and incubated, with shaking, at 37±2° C. until an optical density (at 650 nm) of 0.6-1.6 is reached. This overnight culture will be used for the mutagenicity test and for genotypic confirmation. Genotype tests will be performed as described in the protocol.

For both the dose range and mutagenicity test, a top agar consisting of 0.6% Difco agar in 0.5% NaCl will be melted and a solution of 0.5 mM L-histidine/0.5 mM biotin or 0.5 mM L-tryptophan will be added to the melted top agar at a ratio of 10 mL per 100 mL agar. The supplemented agar will be aliquotted, 2 mL per tube and held at 45-47° C. To prepare the top agar for treatment, 0.1 mL of the test article or control, 0.1 mL of the bacterial culture and 0.5 mL of phosphate buffered saline will be added to the molten agar. The mixture will be briefly vortexed and poured onto a room temperature minimal glucose agar plate (1.5% Difco agar, 2% glucose, in Vogel-Bonner medium E). Metabolic activation will be provided by adding 0.5 mL of the S9 mix in place of the PBS. The plates will be allowed to harden and then incubated 48-72 hours at 37±2° C. All plates will be counted using an automatic image analysis system. Negative control and test article treated plates will also be examined for the presence of a bacterial lawn.

Exogenous Metabolic Activation

The in vitro metabolic activation system used in this assay is comprised of Sprague Dawley rat liver enzymes and a cofactor pool. The enzymes will be contained in a preparation of liver microsomes (S9 fraction) from rates treated with Arochlor to induce the production of enzymes capable of transforming chemicals to more active forms. Immediately prior to use, the S9 will be thawed and mixed with a cofactor pool to contain 5% S9, 5 mM glucose 6-phosphate, 4 mM β-nicotine-adenine dinucleotide phosphate, 8 mM MgCl₂ and 33 mM KCl in a 200 mM phosphate buffer at pH 7.4.

Dose Levels and Replicates

The test article will be tested in triplicate at five dose levels (20.0, 10.0, 5.0, 2.5, and 1.25 mg/mL) along with appropriate vehicle (5% dextrose) and positive controls in the dose range assay. This is equivalent to 2.0, 1.0, 0.5, 0.25, and 0.125 mg/plate.

For the definitive assay, three dose levels will be chosen (10.0, 10.0, and 5.0 mg/mL), which is equivalent to 2.0, 1.0, and 0.5 mg/plate. All treatments, including negative and positive control, will be plated in triplicate against test strains TA97a, TA98, TA100, TA102, TA1535, and WP2-uvrA⁻ in the presence and absence of metabolic activation. These doses will be chosen based on inducing a range of test article toxicity and maximizing the applied dose.

Control Substances

Control substances may be prepared and used in the mutagenicity assay as described in Table 9.

TABLE 9

| Control | Strain | Concentration | |
|---|---|---|---|
| ICR-191 Acridine | TA97a | 1.0 | μg/plate |
| 2-nitrofluorene | A98 | 10.0 | μg/plate |
| Sodium azide | TA100 and | 1.5 | μg/plate |
| | TA1535 | | |
| 1-methyl-3-nitro- | WP2-uvrA⁻ | 4.0 | μg/plate |
| 1-nitrosognanidine | | | |
| 2-aminoanthracene | all strains (except | 10.0 | μg/plate |
| | TA1535) | | |
| 2-aminoanthracene | TA1535 | 1.6 | μg/plate |

Negative (Vehicle) Control

Tester strains will be plated with untreated dextrose solution at the corresponding maximum concentration (0.1 mL), with and without S9. These plates serve as the negative controls and provide information regarding background lawn and revertant colony formation.

Dose Range Assay

The initial dose range assay starts at the maximum concentration of 2.0 mg/plate. The four lower doses to be tested will be diluted in a 1:2 dilution series.

Reverse Mutation Assay

Each separate bacterial strain, with and without S9, is considered a separate experiment with its own concurrent positive and vehicle controls. All plates will be scored with an automated colony counter and a printout of the data was made. The positive controls will consist of direct-acting mutagens and mutagens requiring metabolic transformation. A two-fold or greater increase in reversion rates may be observed for all strains with the appropriate positive control. The negative control article reversion rates for each strain should be within or slightly below the expected ranges from laboratory historical data. An induced positive result for any strain would be demonstrated by at least a two-fold increase in the number of revertant colonies per plate over the negative control values.

### Example 26

### In Vitro Chromosome Aberration Assay in CHO Cells

The Chromosomal Aberration Assay may be one of several in vitro tests that can be used to screen materials for their potential genetic toxicity. Chromosome aberrations are mutations which have been associated with carcinogenesis. Therefore, the chromosome aberration assay is relevant for testing potential mutagens and carcinogens (Galloway et al., Environ. Mut. (1985) 7:1-51; Galloway et al., Environ. Mut. (1987) 10:1-175). This Chromosome Aberration Assay evaluates the potential of the test article extracts to induce damage in Chinese Hamster Ovary Cells (CHO). This test will be conducted in the presence and absence of an exogenous mammalian activation system (S9) over three treatment periods. All negative control treated preparations should demonstrate normal levels of spontaneously occurring aberrations while positive control treated cultures should demonstrate dramatic, dose dependent increases in aberrant chromosomes.

A representative assay to determine whether a test material is clastogenic, i.e., whether it has the capacity to break chromosomes may be designed as follows. Clastogenicity is an important endpoint because it is through chromosomal breakage and inappropriate rejoining that certain oncogenes (e.g., myc) can be activated and certain tumor suppressor genes (e.g., those suppressing retinoblastoma) can be inactivated). In this test, mammalian Chinese Hamster Ovary (CHO) cells will be exposed to the test material and blocked in metaphase using a spindle poison. Visualization of chromosomes will be performed microscopically after hypotonic swelling, fixing and staining the treated CHO cells. Agents found to be capable of inducing chromosome breakage have a high probability of being carcinogens and also have the potential for inducing heritable chromosomal defects.

The CHO-K₁ cell line (ATCC number: CCL-61) is a proline auxotroph with a modal chromosome number of 20 and a population doubling time of 10-14 hours. This system has been shown to be sensitive to the clastogenic activity of a variety of chemicals (Preston et al., Mutation Res. (1981) 87:143-188). CHO cells will be grown and maintained in McCoy's 5A medium supplemented with 10% fetal calf serum, 1% L-glutamine (2 mM), penicillin (100 units/mL), and streptomycin (100 μg/mL). Cultures will be incubated in 5-7% CO₂ with loose caps in a humidified incubator at 37±2° C.

Test Procedures

A stock solution will be prepared at 5 mg/mL. Lower doses will be prepared by dilution in 5% dextrose from this stock immediately prior to use. To minimize any chance of degradation, the test article solutions will be kept on ice after preparation and until just prior to dosing procedures. Cells will be seeded at approximately 1−1.5×10⁶ cells per 75 cm² tissue culture flask in 10 mL fresh medium one day prior to treatment. For treatment, spent medium will be replaced with fresh growth medium and the test article extract, negative or positive control will be added to each flask. Positive controls will be dosed in 0.1 mL volumes to minimize vehicle toxicity. The test article dilutions and negative control will be dosed in 1 mL volumes. Fresh medium will be added to bring the total treatment volume to 10 mL. For the portion of the test with metabolic activation, the S9 activation mix will be added to serum free medium at 1.5%, (v/v) final concentration. All treatments will be carried out in duplicate. The cells will be incubated at 37±2° C. in the presence of the test article extract, the S9 reaction mixture (metabolic activation portion of the study only) and growth medium. The assay will be divided into three treatment periods: 3 hours, 3 hours with S9 activation, and 20 hours.

After the treatment period, all flasks will be evaluated microscopically for gross manifestations of toxicity. i.e., morphological changes in cells or significant cell detachment. All flasks will be washed twice with phosphate buffered saline (PBS). Normal growth medium containing 10% fetal bovine serum (FBS) will be added to the freshly washed cells and the flasks will be returned to the incubator for an additional 14.5-15.5 hours. Microscopic evaluation will be performed immediately prior to harvest. Two hours prior to harvest, 1 μg of colcemid will be added (0.1 vg/mL final concentration) to all flasks to accumulate dividing cells.

The test article extracts will be tested in duplicate at six dose levels (0.5, 0.16, 0.05, 0.016, 0.005, and 0.0016 ml/mL final concentration in culture) along with appropriate vehicle and positive controls.

### Metabolic Activation System

The use of a metabolic activation system is an important aspect for evaluation of a test article, as some compounds exist only in a promutagenic state. That is, they become mutagenic only after being acted upon by an outside metabolic source. In vitro test systems lack this ability to metabolize compounds unless an outside system such as S9 is added.

The in vitro metabolic activation system to be used in this assay may comprise Sprague Dawley rat liver enzymes and an energy producing system necessary for their function (NADP and isocitric acid; core reaction mixture). The enzymes will be contained in a preparation of liver microsomes (S9 fraction) from rats treated with Arochlor 1254 to induce enzymes capable of transforming chemicals to more active forms. The S9 may be purchased from Moltox (Boone, N.C.) and retained frozen at less than −70° C. until use. This S9 fraction will be thawed immediately before use and added to the core reaction mixture.

Cell Fixation, Staining and Scoring

Metaphase cells will be collected by mitotic shake off, swollen with 75 mM KCl, fixed in methanol:glacial acetic acid (3:1 v/v). Cells will be pipetted onto glass slides after resuspension in fresh fixative and air dried. The slides will be labeled with a blind code. Three slides will be prepared from each treatment flask. Slides will be stained with Giemsa and permanently mounted. All slides will be read under blind code with the exception of the high dose positive controls, which are evaluated first to ensure the aberration frequency was adequate. Two hundred cells per dose (100 from each of the duplicate flasks) will be read from each of the doses. One hundred cells will be read from each of the high dose positive controls in accordance with the following definitions and were scored as such.

### Chromatid Type

TG (Chromatid Gap): “Tid Gap”. An achromatic (unstained) region in one chromatid, the size of which is equal to or smaller than the width of a chromatid. These are noted but not usually included in final totals of aberrations, as they may not all be true breaks.

IG (Isochromatid Gap): “Chromosome Gap”. The gaps are at the same locus in both sister chromatids. These are noted but are not usually included in final totals of aberrations, as they may not all be true breaks.

TB (Chromatid Break): An achromatic region in one chromatid, larger than the width of a chromatid. The associated fragment may be partially or completely displaced, or missing.

ID (Chromatid Deletion): Length of chromatid “cut” from midregion of a chromatid resulting in a small fragment or ring lying beside a shortened chromatid or a gap in the chromatid.

TR (Triradial): An exchange between two chromosomes, which results in a three-armed configuration. May have an associated acentric fragment.

QR (Quadriradial): The same as the triradial, but resulting in a four-armed configuration.

CR (Complex Rearrangement): An exchange among more than two chromosomes which is the result of several breaks and exchanges.

TI (Chromatid Interchange): Exchange within a chromosome involving one or both arms.

### Chromosome Type

SB (Chromosome Break): Terminal deletion. Chromosome has a clear break forming an abnormal (deleted) chromosome with an acentric fragment that is dislocated and may remain associated or may appear anywhere in the cell.

DM (Double Minute Fragment): Chromosome interstitial deletion. These appear as small double “dots” or may be paired rings. In some cases, they cannot be distinguished from acentric fragments that result from exchanges or terminal deletions.

D (Dicentric): An exchange between two chromosomes that results in a chromosome with two centromeres. This is often associated with an acentric fragment in which it is classified as Dicentric with Fragment (DF).

MC (Multi-centric Chromosome): An exchange among chromosomes that results in a chromosome with more than two centromeres.

R (Ring): A chromosome that forms a circle containing a centromere. This is often associated with an acentric fragment, in which case it is classified as Ring with Fragment (RF). Acentric rings are also included in this category.

Ab (Abnormal Monocentric Chromosome): This is a chromosome whose morphology is abnormal for the karyotype, and often the result of such things as a translocation or pericentric inversion. Classification used if abnormally cannot be ascribed to, e.g., a reciprocal translocation.

T (Translocation): Obvious transfer of material between two chromosomes resulting in two abnormal chromosomes. When identifiable, scored at “T”, not as “2 Ab”.

### Other

SD (Severely Damaged Cell): A cell with 10 or more aberrations of any type. A heavily damaged cell should be analyzed to identify the type of aberrations and may not have 10 or more, e.g., because of multiple fragments such as those found associated with a tricentric.

PU (Pulverized Chromosome): Despiralized or fragmented chromosome. This may simply be at a different stage of chromosome condensation.

P (+Pulverized Cell): More than one chromosome, up to the whole nucleus, is “pulverized”.

PP (Polyploid Cell): A cell containing multiple copies of the haploid number of chromosomes. Polyploid cells are occasionally observed in normal bone marrow or cell culture. These are recorded but are not included in final totals of structural aberrations.

Control Substances

Control substances are prepared and used in this assay as described in published reports. Positive controls which may be used are: cyclophosphamide—High dose 15 μg/mL; cyclophosphamide—Low dose 5 μg/mL; mitomycin C—High dose 1.0 μg/mL; and citomycin C—Low dose 0.25 μg/mL. For negative (vehicle) control, the CHO cells are treated with the 5% dextrose negative controls with and without S9 activation. These treatments provide information regarding background numbers of aberrant cells.

Assay Validity Evaluation and Statistical Analysis

The total number of aberrations (% CA) of the solvent control culture(s) should fall within 1-14%. High dose positive controls should produce a statistically significant increase in the number of aberrations at the 95% confidence level (p<0.05) as determined by statistical analysis. Analysis of Variance (ANOVA) may be used to identify significant differences between positive and negative control groups or test article and negative control groups. A difference is considered significant when the p value obtained is less than 0.05.

### Example 27

### Safety and Tolerance Determination in Dogs

A representative study for determining the safety and tolerance of compounds at dose levels administered intravenously once daily to beagle dogs for five consecutive days, for example, may be designed as follows. Safety parameters will be monitored through observation, clinical pathology, and microscopic histopathology assessments.

Experimental Design

Table 10 summarizes a representative study. For example, the study will be conducted using three (3) test article and one (1) control article group. The control article will be the solution (5% dextrose in water) used to dilute the test article prior to administration and will be administered at the same volume as the high dose. The test article dosage levels for this study will be approximately 12, 3.8, and 1.2 mg/kg. Test and control articles will be administered once by intravenous (IV) infusion over approximately a one hour period on five consecutive days.

Blood samples for test article blood level analysis will be taken as follows (i.e., pk/tk sampling). Approximately 1.0 mL of blood will be taken from three male and three female dogs in the low dose group at approximately 20 minutes and 40 minutes from the start of the infusion, and then at the end of infusion (Time 0) and at 5, 10, 15, and 30 minutes, and 1, 2, 4, 8, 12, and 24 hours from the end of the infusion after the first and fifth doses. Also, prior to and immediately after Dose 1 and after Dose 5 for all animals, and for recovery animals prior to necropsy, approximately 5-10 second ECG tracings in a lead II configuration will be obtained. Animals will be terminated one (1) or 15 days after the last dose. Blood for hematology and clinical chemistry analysis will be drawn pre-dose and prior to euthanasia at termination. Following euthanasia, a necropsy will be performed to include collection of major organs for microscopic evaluation.

TABLE 10

| | | | PRIMARY | RECOVERY |
|---|---|---|---|---|
| | | | No. ANIMALS | (15 DAY) |
| GROUP | | DOSAGE | (MALE/ | No. ANIMALS |
| No. | ARTICLE^{a} | (MG/KG) | FEMALE) | (MALE/FEMALE) |
| 1 | Control | 0.0 | 3/3 | 1/1 |
| 2 | Test Article | 12.0 | 3/3 | 1/1 |
| 3 | Test Article | 3.8 | 3/3 | 1/1 |
| 4 | Test Article | 1.2 | 3/3 | 1/1 |

^{a}Delivered as an approximate 1 hour infusion

Test Methods

In a representative study, animals will be assigned to groups as follows: The heaviest dog for a sex will be assigned to Group 1, the next heaviest for that sex will be assigned to Group 2, the next heaviest to Group 3, the next heaviest to Group 4, then continue with Groups 2, 3, 4, and 1, then Groups 3, 4, 1, and 2, continuing with this pattern until each group had a full complement of animals. The test and control article will be administered at each dosing as an intravenous infusion into a cephalic or saphenous vein over approximately one hour.

Animals will be weighed daily prior to dosing and prior to necropsy. All animals will be observed for signs of pharmacological activity, behavioral changes, and toxicity immediately and one hour after dosing. Recovery animals will be also observed once daily during the recovery period. Prior to and immediately after Doses 1 and 5 for all animals, and for recovery animals prior to necropsy, approximately five second ECG tracings in a lead II configuration will be obtained. These tracings will be used to provide data for interpretation of the rhythm and amplitude changes of the QRS-complex and T-wave and to measure QT intervals on a number of segments per tracing (approximately 5-10).

Blood Collection

PK/TK. Blood samples for test article blood level analysis will be taken. Approximately 1 mL of blood will be taken from three males and three females in the low dose group at approximately 20 minutes and 40 minutes from the start of the infusion, and then at the end of infusion (Time 0) and at 5, 10, 15, and 30 minutes, and 1, 2, 4, 8, 12, and 24 hours from the end of the infusion after the first and fifth dose. Plasma (lithium heparin anticoagulant) samples will be prepared for analysis.

Clinical Pathology. After overnight fasting and prior to the first dose (baseline; all animals) and then prior to each necropsy, blood samples will be taken for hematology and clinical chemistry. For hematology assays, blood collected at baseline and prior to necropsy (fasted) are analyzed for erythrocyte count, hematocrit, MCH, leukocyte count, differential WC, MCHC, hemoglobin, MCV, platelet count, PT, and APTT. For clinical chemistry assays, blood collected at baseline and prior to necropsy (fasted) will be tested for: aspartate aminotransferase (ASP), globulin & A/G ratio, Alanine aminotransferase (ALT), sodium, alkaline phosphatase, potassium, gamma glutamyltransferase (GGT), chloride, glucose, calcium, blood urea nitrogen (BUN), total bilirubin, creatinine, inorganic phosphorus, total protein, cholesterol, albumin, and triglycerides.

Necropsy

Following blood sample collection, primary treatment and recovery group animals will be sacrificed at their respective termination times and are necropsied. Major organs will be collected, weighed, and preserved for microscopic evaluation. Necropsy will include examination of the cranial, thoracic, abdominal and pelvic cavities, their viscera, the tissues, organs, and the carcass.

Statistical Methods

Statistical analysis of the clinical chemistry and hematology values and organ and body weight data will be performed to compare the test article groups to the control group. The statistical methods used for the data will be selected as appropriate: parametric data will be analyzed using a one way Analysis of Variance, non-parametric data will be analyzed using the Kurskai-Wallis test. A paired t-test will also be used to compare baseline and post treatment clinical chemistry and hematology values for each animal. Probability (p) values of 0.05 or less will be considered significant for all statistical tests.

### Example 28

### Safety and Tolerance Study in Rats

A representative study to determine the safety and tolerance of a test compound, for example, at three dose levels administered intravenously once daily to rats for five consecutive days may be designed as follows. Safety parameters will be monitored through observation, clinical pathology, and microscopic histopathology assessments. Selected animals will also undergo blood sample collection for pharmacokinetic/toxicokinetic evaluation.

Experimental Methods

Table 11 summarizes a representative study. The study will be conducted using three (3) test and one (1) control article groups. The high and low test article groups and the control group will consist of 28 animals each and will be used to assess tolerance. The medium test article group will consist of 64 animals, of which 28 animals will be used to assess tolerance and 36 animals will be used to determine the level of test article in the blood at various time points after the first and fifth doses in the PK/TK portion of the study. The control article will be the solution (5% dextrose in water; D5W) used to dilute the test article prior to administration and is administered at the same volume as the high dose test article group. The test article dosage levels for this study will be 24, 7.6, and 2.4 mg/kg. Test and control articles will be administered by intravenous (IV) injection into a tail vein over one minute on five consecutive days.

Blood samples for test article blood level analysis will be taken as follows. Approximately 0.3-0.5 mL of blood will be taken from three male and three female rats under anesthesia at each sample time point of pre-dose and at the end of injection (Time 0) and at approximately 0.08, 0.25, 0.5, 1, 2, 4, 8, 12, and 24 hours from the end of the injection after the first and fifth doses. Animals used to assess tolerance will be terminated one day (for the primary group) or 15 days (for the recovery group) after the last dose. At termination of the tolerance test animals, blood for hematology and clinical chemistry analysis will be drawn prior to euthanasia and following euthanasia. A necropsy will be performed to include collection of major organs for microscopic evaluation. The animals used for the pk/tk blood sampling only to determine the level of test article will be euthanized after the final blood sample is collected without any further sampling or observations.

TABLE 11

| | | | PRIMARY | RECOVERY |
|---|---|---|---|---|
| | | | No. ANIMALS | (15 DAY) |
| GROUP | | DOSAGE | (MALE/ | No. ANIMALS |
| No. | ARTICLE^{a} | (MG/KG) | FEMALE) | (MALE/FEMALE) |
| 1 | Control | 0.0 | 3/3 | 1/1 |
| 2 | Test Article | 12.0 | 3/3 | 1/1 |
| 3 | Test Article | 3.8 | 3/3 | 1/1 |
| 4 | Test Article | 1.2 | 3/3 | 1/1 |

^{a}Delivered as an approximate 1 hour infusion

Test Methods

The test and control article will be administered at each dosing as an intravenous infusion into a tail vein over approximately one minute. Animals will be weighed daily prior to dosing and prior to necropsy. All animals will be observed for signs of pharmacological activity, behavioral changes, and toxicity immediately and one hour after dosing. Recovery animals will also be observed once daily during the recovery period. The control animals will be dosed with approximately 6 mL/kg of D5W. The high, mid, and low dose test article animals will be administered dosages of approximately 24 mg/kg, 7.6 mg/kg, and 2.4 mg/kg, respectively.

Blood Collection

PK/TK. Blood samples for test article blood level analysis will be taken. Utilizing 18 male and 18 female medium dose animals, approximately 0.3-0.5 mL of blood will be taken from three male and three female rats under anesthesia at each sampling time point of pre-dose and at the end of injection (Time 0), and at approximately 0.08, 0.25, 0.5, 1, 2, 4, 8, 12, and 24 hours from the end of the injection after the first and fifth dose. Blood sampling will be via retro-orbital bleeding or cardiac puncture bleeding for an animal's terminal sample. Plasma (lithium heparin anticoagulant) samples will be prepared for analysis. General procedures for chemical pathology, necropsy, and histopathology, as well as statistical methods, such as those previously described, will be followed.

### Example 29

### Phosphorylated and Total p53 Assay Protocol

A phosphorylated and total p53 assay protocol may be designed as follows. On Day 1, cells are seeded at 2×10⁶ cells/10 cm dish/10 mL medium. On day two, cells will be treated as follows: control=0.05% DMSO (5 μl DMSO stock/10 ml medium); 1 μM test compound (1 μl Stock (10 mM)/10 ml medium); 2 μM test compound (20 Stock (10 mM)/10 ml medium); 3 μM test compound (3 μl Stock (10 mM)/10 ml medium); 4 μM test compound (4 μl Stock (10 mM)/10 ml medium) and 5 μM test compound (5 μl Stock (10 mM)/10 ml medium).

On Day 3, cells will be harvested and attached and floating cells will be collected. Cells will be washed twice with PBS, counted and collected at 4×10⁶ cells/sample. The cell pellet will be frozen at −80° C. until further use. On the same day or on Day 4, cells will be extracted using a cell extraction buffer (3 mL cell extraction buffer, 300 μl protease inhibitor and 10 μl 0.3M PMSF). To each sample will be added 200 μl Buffer, and the solution will be vortexed and set on ice for 30 minutes, and subsequently vortexed after every 10 mins. The solution will be then centrifuged at 13,000 rpm for 10 min, and 100 μl supernatant per tube will be aliquoted and stored at −80° C.

Assay preparation (Day 5). An anti-rabbit IgG HRP solution will be prepared by diluting 10 μl of 100× concentrate solution with 1 ml HRP diluent for each 8-well strip. A wash buffer solution will be prepared by diluting the original vial (×25) using distilled water to make a ×1 solution. Dilutions of p53 standard solution or p53 total solution can be prepared as described according to representative parameters of Table 12. To ensure complete reconstitution, standard 1 will be mixed gently and allowed to sit for 10 minutes at room temperature.

TABLE 12

| | Conc. | | Standard Soln. | Dilution Buffer |
|---|---|---|---|---|
| Standard 1 | 100 | Units/ml | Reconstitute 1 Vial worth 0.7 ml of | |
| | | | standard Dil. Buffer | |
| Standard 2 | 50 | Units/ml | 250 μl of Standard 1 | 250 μl |
| Standard 3 | 25 | Units/ml | 250 μl of Standard 2 | 250 μl |
| Standard 4 | 12.5 | Units/ml | 250 μl of Standard 3 | 250 μl |
| Standard 5 | 6.25 | Units/ml | 250 μl of Standard 4 | 250 μl |
| Standard 6 | 3.12 | Units/ml | 250 μl of Standard 5 | 250 μl |
| Standard 7 | 1.6 | Units/ml | 250 μl of Standard 6 | 250 μl |
| Standard 8 | 0 | | | 250 μl |

Test Procedure. Allow all solution to reach RT and mix gently before use. Take out and insert 8-well strips. Add 100 μl of standard dilution buffer to standard 8 well (0 ng/ml/well or 0 Units/well). Add nothing to the chromogen blank well. Add 100 μl of standard or diluted sample to the appropriate microtiter wells. Generally, the sample should be diluted with standard dilution buffer at least 1:10 or greater. Each sample will be run in duplicates. Gently tap the side of the plate to thoroughly mix. Cover plate with plate cover and incubate for 2 hours at RT or o/n at 4 C. Wash wells with 400 μl working wash buffer 4 times. Let soak for 15-30 sec., and then aspirate the liquid. After washing, the plate will be inverted and tapped dry on absorbance tissue. Add 100 μl of anti-p53 [pS15] or anti-p53 (total) (detection antibody) to each well except chromogen blank. Tap gently to mix; cover plate and incubate 1 hour at RT. Aspirate solution from wells thoroughly.

Wash wells with 400 μl working wash buffer four times. Let soak for 15-30 sec., and then aspirate the liquid. After washing, the plate will be inverted and tapped try on absorbance tissue. Add 100 μl of anti-rabbit IgG HRP working solution. to each well except chromogen blank. Cover plate and incubate 30 min at RT. Wash wells with 400 μl working wash buffer four times. Let soak for 15-30 sec., and then aspirate the liquid. After washing, the plate will be inverted and tapped try on absorbance tissue. Add 100 μl of TMB (stabilized chromogen substrate) to each well and incubate for 30 min. at RT in the dark. The color will change to blue. Add 100 μl Stop soln. Tap plate gently to mix. The color should change to yellow. Read the plate at A450 nm by setting chromogen blank (=100 μl TMB+100 μl Stop soln) as blank. Read absorbance within 2 hours of assay completion.

### Example 30

### Caspase-3/7 Assay Protocol

A representative Caspase-3/7 assay protocol may be designed as follows. On Day 1, seed 0.015×10₆ HCT-116 cells/50 ul/well. Incubate o/n in 37° C. CO₂ incubator. On Day 2, remove 25 ul of medium from wells. Treat HCT-116 cells with 1, 3, and 5 uM test compound. Treat positive control group with Staurosporin 0.01, 0.1, 1 uM. Keep six negative control wells treated with medium only (add 25 ul of diluted sample to appropriate wells). Incubate for 24 h at 37° C. in a CO₂ incubator. On Day 3, prepare Apo-ONE Homogeneous Caspase-3/7 assay reagent (Promega) at 10 ul reagent/1 ml buffer. Add 50 ul of diluted reagent. Incubate one hour at room temp. Measure fluorescence at 485/520.

### Example 31

### Annexin V-Alexa 488 Staining Protocol

A representative Annexin V-Alexa 488 staining protocol may be designed as follows. Seed 1.5±2.0×10⁶ HCT-116 cells/10 cm dish/10 ml medium. Incubate o/n or up to 24 hrs at 37° C. in CO₂ incubator. The following day, treat cells with 1, 2, 3, 4 and 5 μM test compound. Keep one or two untreated plates (medium only) as control plates. The following controls are used: untreated samples (no Alexa or propidium iodide), controls treated with propidium iodide or Alexa 488 only, and controls treated with both Alexa 488 and propidium iodide. Harvest cells (collect attached as well as floating cells). Wash cells twice with cold PBS. Re-suspend cells in 1× Annexin binding buffer.

Count cells and dilute in 1× Annexin binding buffer to ˜1×10⁶ cells/0.1 ml, preparing a sufficient volume to have 100 μl per assay. Add 5 μl of the Annexin V conjugate to each 100 μl of cell suspension. Add 4 μl of propidium iodide solution (stock=1 mg/ml) to each 100 μl of cell suspension. Incubate sample at RT for 15 minutes. Add 400 μl Annexin binding buffer, mix gently and keep samples on ice. Analyze stained cells immediately by flow cytometry.

### Example 32

### DNA Cell Cycle Analysis Protocol

A representative DNA cell cycle analysis protocol will be designed as follows. Seed 1.5-2.0×10⁶ cells/10 cm dish (seed one extra dish for unstained cells). Incubate cells in 37° C. humidified 5% CO₂ incubator for 24 hours. For synchronizing cells in a low growth state to make cells quiescent, remove media and rinse once with serum-free media, add 10 ml of serum-free media to each dish. Incubate the cells for 24 hr in a 37° C. humidified 5% CO₂ incubator. Remove media and add treatment (diluted in serum contained media, 10 ml): 1-5 μM test compound plus control. Incubate the cells for 24 hr in a 37° C. humidified 5% CO₂ incubator.

To trypsinize/isolate cells, remove treatment. Add 3 ml trypsin/EDTA solution. Keep floating cells and combine with attached cells. Incubate for 5 min in a 37° C. humidified 5% CO₂ incubator. Add 3 ml media (containing FBS) to wells and pipette into centrifuge tube. Centrifuge at 1000 rpm for 5 minutes. Decant supernatant and re-suspend pellet in 2-3 ml PBS. Count cells and wash cells once by putting 2×10⁶ cells/tube, adding 2 ml PBS and centrifuging at 1000 rpm for 5 minutes. Re-suspend pelleted cells in 0.3 ml cold PBS.

To fix cells, gently add 0.7 ml ice cold 70% ethanol drop wise to tube containing 0.3 ml of cell suspension in PBS while vortexing. Leave on Ice for one hour (or up to a few days at 4 C). Centrifuge at 1000 rpm for 5 minutes. Wash one time with cold PBS (1-2 ml). Centrifuge at 1000 rpm for 5 minutes. Re-suspend cell pellet in 0.25 ml cold PBS, add 5 μl of 10 mg/ml RNAse A (the final concentration being 0.2-0.5 mg/ml). Incubate at 37 C for 1 hour. Add 100 of 1 mg/ml of propidium iodide solution in deionized water (the final concentration being 100 μl/ml), and keep in the dark and at 4° C. until analysis. Analyze on FACS by reading on cytometer at 488 nm. Cells may be stained with propidium iodide on the same day of analysis.

It is understood that the foregoing detailed description and accompanying examples are merely illustrative, and are not to be taken as limitations upon the scope of the invention. Various changes and modifications to the disclosed embodiments will be apparent to those skilled in the art. Such changes and modifications, including without limitation those relating to the chemical structures, substituents, derivatives, intermediates, syntheses, formulations and/or methods of use of the invention, may be made without departing from the spirit and scope thereof. U.S. patents and publications referenced herein are incorporated by reference.

1. A compound of formula (1) or (2) or (3), 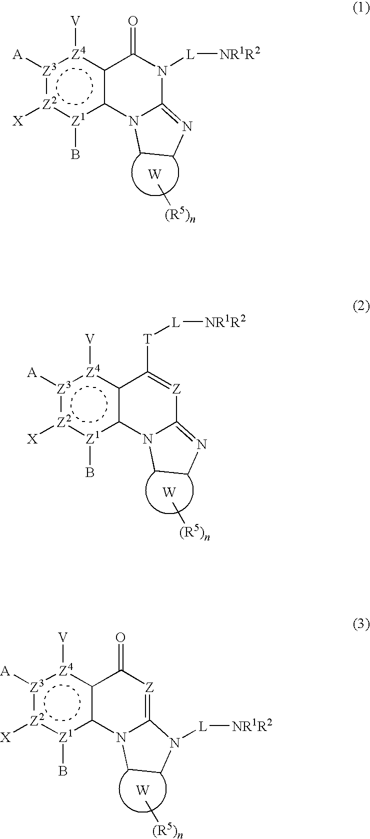
wherein each A, V, B, and X that is present is independently selected from H, halo, azido, CN, CF₃, CONR¹R², R², CH₂R², SR², OR²C(═O)R², and NR¹R², wherein in NR¹R², R¹ and R² can optionally cyclize to form an optionally substituted azacyclic group; or
wherein A and X, or A and V, or X and B may form a carbocyclic ring, heterocyclic ring, aryl or heteroaryl ring, each of which may be optionally substituted with one or two R³ groups and/or may be fused with an additional ring;
wherein in L-NR¹R², R¹ and R² taken together may form an optionally substituted azacyclic group, or R¹ or R² taken together with at least a portion of L may form an optionally substituted heterocyclic ring;
each Z is independently CH, CR³ or N;
each Z¹, Z², Z³, and Z⁴ is independently C or N, provided no two of them represent adjacent nitrogen atoms;
T is O, S(O)ₘ or NR⁴;
each R¹ is H or a C₁₋₁₀ alkyl or C₂₋₁₀ alkenyl group that can be substituted with one or more substituents selected from halo, ═O, OR², NR²₂, S(O)ₘR², COOR², and CONR²₂;
each m is independently 0-2;
each n is independently 0-4;
each R² is independently H or an optionally substituted C₁₋₁₀ alkyl or optionally substituted C₂₋₁₀ alkenyl optionally containing one or more non-adjacent heteroatoms selected from N, O, and S in place of carbon atoms, and optionally including a carbocyclic or heterocyclic ring; or R² is an optionally substituted carbocyclic, heterocyclic, 6-10 membered aryl or 5-14 membered heteroaryl ring containing one or more N, O or S;
each R³ is independently an optionally substituted group selected from C₁₋₆ alkyl, C₆₋₁₀ aryl, and C₅₋₁₂ heteroaryl, or R³ is selected from halo, nitro, OR′, SR′, SO₂R′, NR′₂, CN, CF₃, COOR′, and CONR′₂, wherein each R′ is independently H or C₁₋₆ alkyl and can optionally include one N, O or S in place of a carbon atom, or R³ can be L-NR¹R² or CON(R′)-L-NR¹R², wherein in NR¹R², R¹ and R² can optionally cyclize to form an optionally substituted azacyclic group;
each R⁴ is H or a C₁₋₁₀ alkyl or C₂₋₁₀ alkenyl group that can be substituted with one or more substituents selected from halo, ═O, OR², NR²₂, S(O)ₘR², COOR², and CONR²₂;
each L is a divalent hydrocarbon linker having up to ten atoms counted along the shortest path between the two open valence, which linker may include one or two heteroatoms and may be substituted by one or more groups selected from halo, ═O, C₁₋₆ alkyl, OR′, SR′, SO₂R′, NR′2, CN, CF₃, COOR′, and CONR′2, wherein each R′ is independently H or C₁₋₆ alkyl;
each W represents an optionally substituted aryl or heteroaryl ring, which may be a monocyclic group with 5-6 ring atoms, or may be a 5-6 membered ring that is fused with or bonded to one or more additional aryl, heterocyclic, or heteroaryl rings; and
each R⁵ is a substituent at any position on W, and is selected from H, halo, CN, CF₃, OR², NR¹R², and C₁₋₆ alkyl and C₂₋₆ alkenyl, each optionally substituted by one or more substituents selected from halo, ═O, OR², S(O)ₘR², and NR¹R², wherein in NR¹R², R¹ and R² can optionally cyclize to form an optionally substituted azacyclic group; or R⁵ can be an inorganic substituent; or two adjacent R⁵ may be linked to form a 5-6 membered substituted or unsubstituted carbocyclic or heterocyclic ring, optionally fused to an additional substituted or unsubstituted carbocyclic or heterocyclic ring;
or a pharmaceutically acceptable salt thereof.

2-4. (canceled)

5. The compound of claim 1, wherein Z¹ is N.

6. The compound of claim 1, wherein Z² is C and X is an optionally substituted azacyclic group.

7. The compound of claim 1, wherein, in L-NR¹R² shown in the formula, L is (CH₂)₂₋₄ and NR¹R² represents an optionally substituted azacyclic group, or -L-NR¹R² shown in the formula represents a group of formula (4): 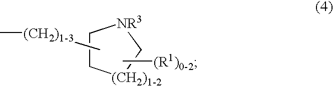
wherein R¹ and R² are as defined in claim 1, and the substituents R¹, if present, and the attachment point for the alkylene linker —(CH₂)₁₋₃— can be at any position on the ring other than the nitrogen atom.

8. The compound of claim 1, which is a compound of formula (1) wherein W represents an optionally substituted phenyl ring.

9. The compound of claim 1, wherein three of Z¹-Z⁴ represent C and one of Z¹-Z⁴ represents N.

10. The compound of claim 1, wherein at least one of V, A, B, and X comprises an azacyclic group.

11. The compound of claim 1, wherein Z¹ is N, Z²-Z⁴ each represent C, and X represents NR¹R², or X comprises an azacyclic group.

12. The compound of claim 11, wherein L is (CH₂)₂₋₄.

13. The compound of claim 1, wherein W in any compound having formula (1), (2), or (3) is selected from the group consisting of: 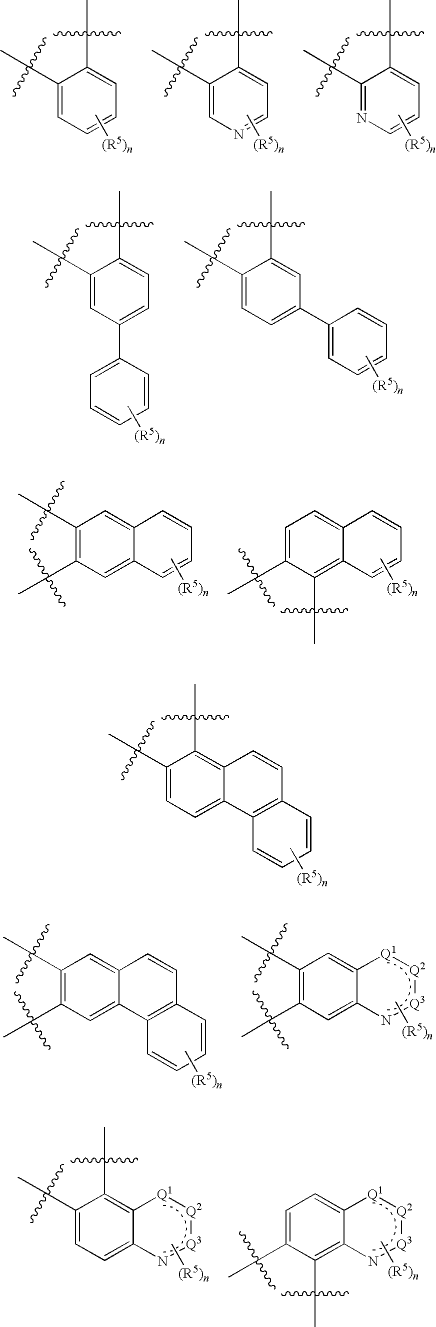 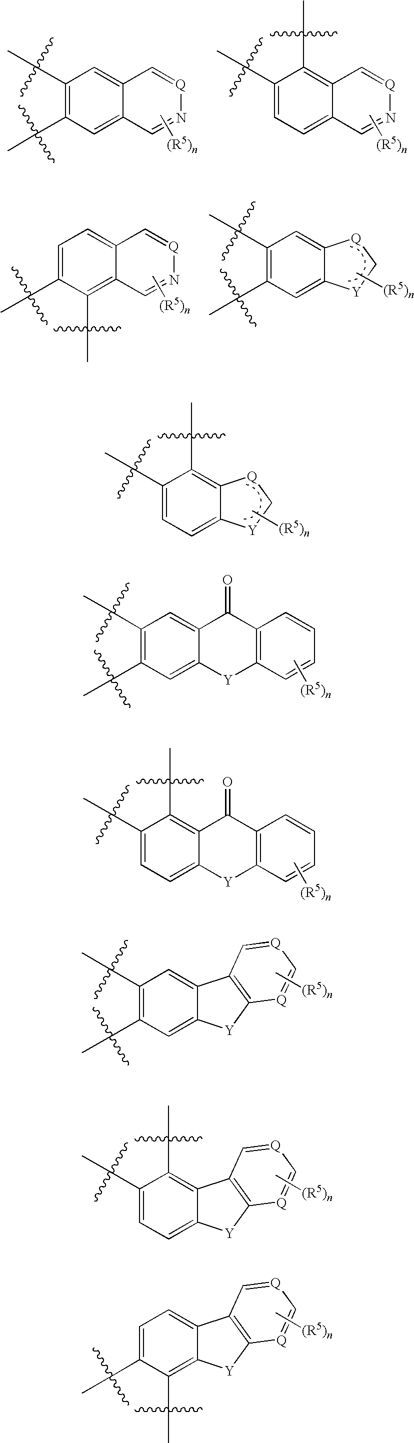 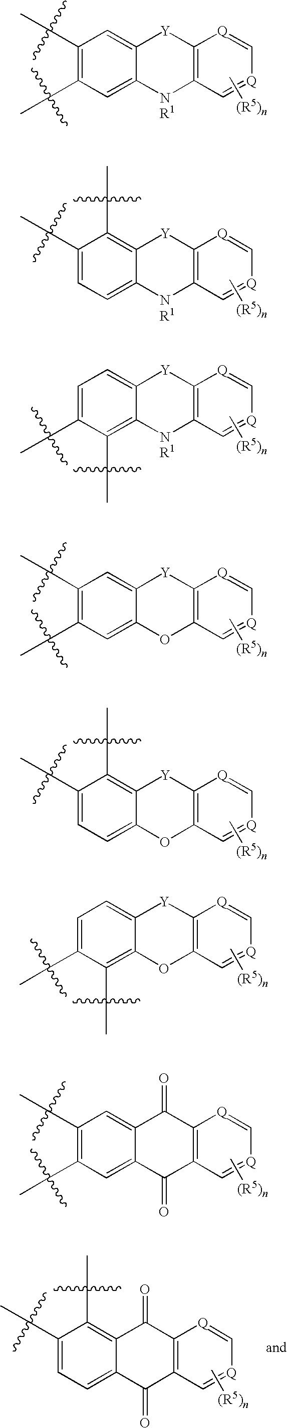 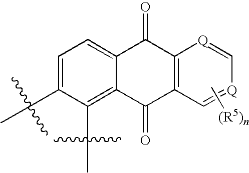
wherein each Q, Q¹, Q², and Q³ is independently CR³ or N;
each Y is independently O, CR¹₂, C═O or NR¹; and
n, R¹, R³ and R⁵ are as defined above.

14. The compound of claim 13, which is of the formula (1a), (2a), or (3a).

15. The compound of claim 3, wherein T is NR⁴.

16. The compound of claim 1, which is a compound of formula (2) or formula (3), wherein Z is CH or N, and L is (CH₂)₂₋₄.

17. The compound of claim 16, wherein Z¹ is N, Z²-Z⁴ each represent C, and X represents NR¹R².

18. The compound of claim 17, wherein W represents an optionally substituted phenyl ring.

19. The compound of claim 1, wherein L in L-NR¹R² represents (CH₂)₃, and NR¹R² mL-NR¹R² represents an azacyclic group.

20. The compound of claim 10, wherein L in L-NR¹R² represents (CH₂)₃, and NR¹R² in L-NR₁R₂ represents an azacyclic group.

21. The compound of claim 20, wherein W represents an optionally substituted phenyl ring.

22. A method to treat a proliferative disorder or a bacterial infection, which method comprises administering to a subject in need of such treatment, an effective amount of a compound according to claim 1.

23-26. (canceled)

27. A pharmaceutical composition comprising a compound according to claim 1 and at least one pharmaceutically acceptable excipient.

28-29. (canceled)

30. A method to treat a condition associated with overexpression of an oncogene, said method comprising administering to a cell in an in vitro or in vivo environment, an effective amount of a compound according to claim 1.

31. A method to identify a molecule that modulates protein kinase activity, said method comprising screening a compound according to claim 1, or a library of compounds according to claim 1, to identify a compound having an effect on the activity of a protein kinase.

32. (canceled)
